(19) United States
(12) Patent Application Publication
(10) Pub. No.: US 2023/0295312 A1
(43) Pub. Date: Sep. 21, 2023

(54) ANTIBODY AGAINST HUMAN IL-4RA AND USE THEREOF

(71) Applicant: Akeso Biopharma, Inc, Guangdong (CN)

(72) Inventors: Baiyong Li, Guangdong (CN); Yu Xia, Guangdong (CN); Zhongmin Wang, Guangdong (CN); Peng Zhang, Guangdong (CN)

(21) Appl. No.: 17/419,109

(22) PCT Filed: Dec. 27, 2019
(86) PCT No.: PCT/CN2019/129193

§ 371 Date: Dec. 14, 2021

(30) Foreign Application Priority Data
Dec. 27, 2018 (CN) 201811618948.8

(51) Int. Cl. C07K 16/28

(52) U.S. Cl. CPC C07K 16/2866; C07K 2317/24; C07K 2317/31; C07K 2317/55; C07K 2317/565

(57) ABSTRACT

The present invention relates to an anti-human interleukin-4-receptor A antibody, a pharmaceutical composition or a kit comprising the antibody, and use thereof.

### CROSS-REFERENCE TO RELATED APPLICATIONS

This application is a National Phase application under 35 U.S.C. 371 of PCT/CN2019/129193, filed Dec. 27, 2019, and claims the benefit of and priority to Chinese Application No. 201811618948.8, filed Dec. 27, 2018, the disclosures of which are hereby incorporated herein by reference in their entirety.

### CROSS REFERENCE TO SEQUENCE LISTING

The Sequence Listing submitted Jan. 26, 2022, as a text file named “ABIO_100_ST25.txt” created on Jan. 7, 2022, and having a size of 70,545 bytes is hereby incorporated by reference.

### TECHNICAL FIELD

The present invention relates to the field of allergic disease treatment and molecular immunology, and particularly, to an anti-human interleukin-4-receptor A antibody, a pharmaceutical composition or a kit comprising the same, and use thereof.

### BACKGROUND

Interleukin-4-receptor (IL-4R) is a transmembrane receptor that exists in two distinct forms: type I IL-4R, which consists of an IL-4R a subunit (referred to as IL-4RA herein) with high affinity and a γ_{c} subunit with moderate affinity, and binds to interleukin-4 (IL-4) and mediates IL-4 induced cell proliferation, activation and other biological functions; and type II IL-4R, which consists of an IL-4R a subunit (IL-4RA) with high affinity and an interleukin-13-receptor a subunit (IL-13Ra), and is a homologous functional receptor for interleukin-13 (IL-13) capable of binding to IL-13 (Wang and Secombes, Cytokine, 2015, 75(1):8-13). IL-4R is expressed in T cells, B cells, hematopoietic stem cells and endothelial cells, epithelial cells, muscle, fibroblasts, hepatocytes, and brain tissue. IL-4RA, along with γ_{c} subunit or IL-13R a subunit, activates various non-receptor protein tyrosine kinases in cytoplasm after IL-4 binds to its receptor, further initiating downstream signal transduction pathways (Nelms et al., Annu. Rev. Immunol., 1999, 17:701-738; LaPorte et al., Cell, 2008, 132(2):259-272).

IL-4RA can bind to IL-4 and IL-13 with high affinity, and is a major functional subunit of the foregoing type I and type II IL-4R. The inhibition of IL-4RA can effectively block relevant biological functions mediated by IL-4 and IL-13 (Gessner et al., Immunobiology, 2000, 201:285).

IL-4 is a pleiotropic cytokine secreted by immune cells such as CD4⁺ T cell subsets, B cells, mast cells and the like; IL-13 is produced mainly by activated T cells (Th2 in mice) in human, and has various biological effects on monocytes (macrophages), B lymphocytes, NK cells, vascular endothelial cells and the like. In vitro studies have shown that IL-4 and IL-13 can exert corresponding effector functions in a variety of cells, such as T cells, B cells, eosinophils, mast cells, basophils, airway smooth muscle cells, respiratory tract epithelial cells, fibroblasts and endothelial cells, which are the major effector cells responsible for the development of allergic diseases such as allergic rhinitis and food allergy, and asthma (May et al., Cytokine, 2015, 75(1):89-116); furthermore, airway smooth muscle cells, respiratory tract epithelial cells, fibroblasts and endothelial cells, etc. are also involved in the development, progression and maintenance of chronic obstructive pulmonary disease (for reviews, see Steinke et al., RespRes, 2001, 2(66):66-70 and Willis-Karp et al., Immunol Rev, 2004, 202:175-190); T and B cell dysfunctions are the major mechanisms of autoimmune diseases, and IL-4 is involved in the development of autoimmune diseases by activating downstream signaling pathways through binding to IL-4R on the surface of immune cells (May et al., Cytokine, 2015, 75(1):89-116).

The IL-4/IL-13 pathways play an important role in asthma pathology (Chatila et al., Trends in Molecular Med., 2004, 10(10):493-499) as well as in other diseases outlined elsewhere herein. Airway hyperreactivity, mucus0 hypersecretion, and airway remodeling, etc. are the key pathological features of asthma. Studies have demonstrated that IL-4 and IL-13 are involved in the development and maintenance of the above pathological processes (May et al., Cytokine, 2015, 75(1):89-116). IL-13 is considered as a key cytokine for triggering airway hyperreactivity (AHR), and IL-4 is a major cause of Th2 immune cell polarization and IgE production (Wynn et al., Annu. Rev. Immunol., 2003, 21:425-456).

Atopic dermatitis, especially moderate and severe atopic dermatitis, is a serious chronic inflammatory skin disease, mainly characterized by severe itching, pronounced eczematous changes and dry skin. Atopic dermatitis usually starts in infants, and continues for a lifetime in some patients. It may seriously affect the quality of life of the patients due to chronic recurrent eczematous rash, severe itching, sleep deficit, dietary restrictions, and psychosocial reasons. IL-4/IL-13 are considered as a key driver of the persistent intrinsic inflammation of atopic dermatitis (Malajian et al., Cytokine, 2015, 73(2):311-318). Clinical studies have demonstrated that dupilumab, an anti-human IL-4RA monoclonal antibody, is effective in treating moderate and severe atopic dermatitis (Beck et al., N. Engl. J. Med., 2014, 371(2):130-139) and has been approved by the U.S. Food and Drug Administration (FDA) for treating moderate and severe atopic dermatitis.

In addition, basic medical researches and clinical researches also proved that IL-4, IL-13 and IL-4R pathways are involved in the development and progression of chronic obstructive pulmonary disease, rhinosinusitis and tumors, and the inhibition of IL-4RA has the potential for treating chronic obstructive pulmonary disease (Jin Lin et al., Journal of Practical Medicine, 2014, 30(22):3543-3544), rhinosinusitis, pulmonary fibrosis and tumors (May et al., Cytokine, 2015, 75(1):89-116; Guo Changkuo et al., Chemistry of Life, 2017, 37(3):413-418). In adult patients with symptomatic chronic rhinosinusitis and nasal polyposis refractory to intranasal glucocorticoid, anti-human IL-4RA monoclonal antibody dupilumab in combination with glucocorticoids can significantly ameliorate symptoms and reduce nasal polyps (Bachert et al., JAMA, 2016, 315(5):469-79).

The anti-human IL-4RA antibody medicament has wide application prospect, and can be used for treating allergic diseases, such as allergic rhinitis, asthma, allergy and atopic dermatitis, while treating rhinosinusitis, nasal polyps, chronic obstructive pulmonary disease, tissue fibrosis, tumor, autoimmune diseases and the like. Therefore, the development of antibody medicaments with high affinity to human IL-4RA for treating allergic diseases with better efficacy and less severe toxic and side effects poses great significance. However, available anti-human IL-4RA antibody medicaments showed insufficient affinities, and there’s still a need for an anti-human IL-4RA antibody with high affinity.

### SUMMARY

After intensive studies and creative efforts, the inventors used mammalian cell expression systems to express recombinant IL-4RA as an antigen to immunize mice, and obtained hybridoma cells by fusion of mouse spleen cells and myeloma cells. The inventors obtained the following hybridoma cell lines by screening a large number of the samples.

The inventor found that:

The hybridoma cell line 13E5 is capable of secreting and producing a specific antibody (named as 13E5) that specifically binds to the human IL-4RA, and the antibody can block the binding of human IL-4RA to IL-4 effectively.

Furthermore, the inventors have creatively prepared humanized anti-human IL-4RA antibodies (named as 13E5 H1L1, 13E5 H2L2, 13E5 H3L3, 13E5 H4L4 and 13E5 H4L2, respectively).

The hybridoma cell line 18H7 is capable of secreting and producing a specific antibody (named as 18H7) that specifically binds to the human IL-4RA, and the antibody can block the binding of human IL-4RA to IL-4 effectively.

Furthermore, the inventors have creatively prepared humanized anti-human IL-4RA antibodies (named as 18H7 H1L1, 18H7 H2L2, 18H7 H2L3 and 18H7 H3L2, respectively).

The hybridoma cell line 20G10 is capable of secreting and producing a specific antibody (named as 20G10) that specifically binds to the human IL-4RA, and the antibody can block the binding of human IL-4RA and IL-4 effectively.

Furthermore, the inventors have creatively prepared a humanized anti-human IL-4RA antibody (named as 20G10 H3L3).

The foregoing antibodies can effectively bind to the human IL-4RA, block the binding of human IL-4RA to the ligand IL-4 or IL-13 thereof, and inhibit the activation of the downstream signaling pathway of the human IL-4 RA. The antibodies have potential in preparing a medicament for preventing and treating allergic rhinitis, asthma, allergy, atopic dermatitis, rhinosinusitis, nasal polyps, chronic obstructive pulmonary disease, tissue fibrosis and autoimmune diseases.

The present invention is detailed below.

One aspect of the present invention relates to an antibody or an antigen-binding fragment thereof, the antibody comprising:
an HCDR1, comprising or consisting of the sequence set forth in SEQ ID NO: 9, a sequence having at least 90%, preferably at least 91%, 92%, 93%, 94%, 95%, 96%, 97%, 98% or 99% sequence identity to the sequence, or an amino acid sequence having one or more (preferably 1, 2 or 3) conservative amino acid mutations (preferably substitutions, insertions or deletions) compared to the sequence,
an HCDR2, comprising or consisting of the sequence set forth in SEQ ID NO: 10, a sequence having at least 90%, preferably at least 91%, 92%, 93%, 94%, 95%, 96%, 97%, 98% or 99% sequence identity to the sequence, or an amino acid sequence having one or more (preferably 1, 2 or 3) conservative amino acid mutations (preferably substitutions, insertions or deletions) compared to the sequence, and
an HCDR3, comprising or consisting of the sequence set forth in SEQ ID NO: 11, a sequence having at least 90%, preferably at least 91%, 92%, 93%, 94%, 95%, 96%, 97%, 98% or 99% sequence identity to the sequence, or an amino acid sequence having one or more (preferably 1, 2 or 3) conservative amino acid mutations (preferably substitutions, insertions or deletions) compared to the sequence, and the antibody further comprising:
   an LCDR1, comprising or consisting of the sequence set forth in SEQ ID NO: 12, a sequence having at least 90%, preferably at least 91%, 92%, 93%, 94%, 95%, 96%, 97%, 98% or 99% sequence identity to the sequence, or an amino acid sequence having one or more (preferably 1, 2 or 3) conservative amino acid mutations (preferably substitutions, insertions or deletions) compared to the sequence,
   an LCDR2, comprising or consisting of the sequence set forth in SEQ ID NO: 13, a sequence having at least 90%, preferably at least 91%, 92%, 93%, 94%, 95%, 96%, 97%, 98% or 99% sequence identity to the sequence, or an amino acid sequence having one or more (preferably 1, 2 or 3) conservative amino acid mutations (preferably substitutions, insertions or deletions) compared to the sequence, and
   an LCDR3, comprising or consisting of the sequence set forth in SEQ ID NO: 14, a sequence having at least 90%, preferably at least 91%, 92%, 93%, 94%, 95%, 96%, 97%, 98% or 99% sequence identity to the sequence, or an amino acid sequence having one or more (preferably 1, 2 or 3) conservative amino acid mutations (preferably substitutions, insertions or deletions) compared to the sequence;
   or, the antibody comprising:
   an HCDR1, comprising or consisting of the sequence set forth in SEQ ID NO: 15, a sequence having at least 90%, preferably at least 91%, 92%, 93%, 94%, 95%, 96%, 97%, 98% or 99% sequence identity to the sequence, or an amino acid sequence having one or more (preferably 1, 2 or 3) conservative amino acid mutations (preferably substitutions, insertions or deletions) compared to the sequence,
   an HCDR2, comprising or consisting of the sequence set forth in SEQ ID NO: 16, a sequence having at least 90%, preferably at least 91%, 92%, 93%, 94%, 95%, 96%, 97%, 98% or 99% sequence identity to the sequence, or an amino acid sequence having one or more (preferably 1, 2 or 3) conservative amino acid mutations (preferably substitutions, insertions or deletions) compared to the sequence, and
   an HCDR3, comprising or consisting of the sequence set forth in SEQ ID NO: 17, a sequence having at least 90%, preferably at least 91%, 92%, 93%, 94%, 95%, 96%, 97%, 98% or 99% sequence identity to the sequence, or an amino acid sequence having one or more (preferably 1, 2 or 3) conservative amino acid mutations (preferably substitutions, insertions or deletions) compared to the sequence,
and the antibody further comprising:
   an LCDR1, comprising or consisting of the sequence set forth in SEQ ID NO: 18, a sequence having at least 90%, preferably at least 91%, 92%, 93%, 94%, 95%, 96%, 97%, 98% or 99% sequence identity to the sequence, or an amino acid sequence having one or more (preferably 1, 2 or 3) conservative amino acid mutations (preferably substitutions, insertions or deletions) compared to the sequence,
   an LCDR2, comprising or consisting of the sequence set forth in SEQ ID NO: 19, a sequence having at least 90%, preferably at least 91%, 92%, 93%, 94%, 95%, 96%, 97%, 98% or 99% sequence identity to the sequence, or an amino acid sequence having one or more (preferably 1, 2 or 3) conservative amino acid mutations (preferably substitutions, insertions or deletions) compared to the sequence, and
   an LCDR3, comprising or consisting of the sequence set forth in SEQ ID NO: 20, a sequence having at least 90%, preferably at least 91%, 92%, 93%, 94%, 95%, 96%, 97%, 98% or 99% sequence identity to the sequence, or an amino acid sequence having one or more (preferably 1, 2 or 3) conservative amino acid mutations (preferably substitutions, insertions or deletions) compared to the sequence;
   or, the antibody comprising:
   an HCDR1, comprising or consisting of the sequence set forth in SEQ ID NO: 130, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence,
   an HCDR2, comprising or consisting of the sequence set forth in SEQ ID NO: 131, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence, and
   an HCDR3, comprising or consisting of the sequence set forth in SEQ ID NO: 132, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence,
and the antibody further comprising:
   an LCDR1, comprising or consisting of the sequence set forth in SEQ ID NO: 133, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence,
   an LCDR2, comprising or consisting of the sequence set forth in SEQ ID NO: 134, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence, and
   an LCDR3, comprising or consisting of the sequence set forth in SEQ ID NO: 135, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence.

In one embodiment of the present invention, the antibody comprises:
(1) a heavy chain variable region comprising or consisting of:
   the amino acid sequence set forth in SEQ ID NO: 2,
   a sequence having at least 80%, 85% or 90%, preferably at least 91%, 92%, 93%, 94%, 95%, 96%, 97%, 98% or 99% sequence identity to the sequence set forth in SEQ ID NO: 2, or
   an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 2, and
   a light chain variable region comprising or consisting of:
      the amino acid sequence set forth in SEQ ID NO: 4,
      a sequence having at least 80%, 85% or 90%, preferably at least 91%, 92%, 93%, 94%, 95%, 96%, 97%, 98% or 99% sequence identity to the sequence set forth in SEQ ID NO: 4, or
      an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 4;
(2) a heavy chain variable region comprising or consisting of:
   the amino acid sequence set forth in SEQ ID NO: 6,
   a sequence having at least 80%, 85% or 90%, preferably at least 91%, 92%, 93%, 94%, 95%, 96%, 97%, 98% or 99% sequence identity to the sequence set forth in SEQ ID NO: 6, or
   an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 6, and
   a light chain variable region comprising or consisting of:
      the amino acid sequence set forth in SEQ ID NO: 8,
      a sequence having at least 80%, 85% or 90%, preferably at least 91%, 92%, 93%, 94%, 95%, 96%, 97%, 98% or 99% sequence identity to the sequence set forth in SEQ ID NO: 8, or
      an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 8;
(3) a heavy chain variable region comprising or consisting of:
   the amino acid sequence set forth in SEQ ID NO: 22,
   a sequence having at least 80%, 85% or 90%, preferably at least 91%, 92%, 93%, 94%, 95%, 96%, 97%, 98% or 99% sequence identity to the sequence set forth in SEQ ID NO: 22, or
   an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 22, and a light chain variable region comprising or consisting of:
      the amino acid sequence set forth in SEQ ID NO: 24,
      a sequence having at least 80%, 85% or 90%, preferably at least 91%, 92%, 93%, 94%, 95%, 96%, 97%, 98% or 99% sequence identity to the sequence set forth in SEQ ID NO: 24, or
      an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 24;
(4) a heavy chain variable region comprising or consisting of:
   the amino acid sequence set forth in SEQ ID NO: 26,
   a sequence having at least 80%, 85% or 90%, preferably at least 91%, 92%, 93%, 94%, 95%, 96%, 97%, 98% or 99% sequence identity to the sequence set forth in SEQ ID NO: 26, or
   an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 26, and a light chain variable region comprising or consisting of:
      the amino acid sequence set forth in SEQ ID NO: 28,
      a sequence having at least 80%, 85% or 90%, preferably at least 91%, 92%, 93%, 94%, 95%, 96%, 97%, 98% or 99% sequence identity to the sequence set forth in SEQ ID NO: 28, or
      an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 28;
(5) a heavy chain variable region comprising or consisting of:
   the amino acid sequence set forth in SEQ ID NO: 30,
   a sequence having at least 80%, 85% or 90%, preferably at least 91%, 92%, 93%, 94%, 95%, 96%, 97%, 98% or 99% sequence identity to the sequence set forth in SEQ ID NO: 30, or
   an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 30, and a light chain variable region comprising or consisting of:
      the amino acid sequence set forth in SEQ ID NO: 32,
      a sequence having at least 80%, 85% or 90%, preferably at least 91%, 92%, 93%, 94%, 95%, 96%, 97%, 98% or 99% sequence identity to the sequence set forth in SEQ ID NO: 32, or
      an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 32;
(6) a heavy chain variable region comprising or consisting of:
   the amino acid sequence set forth in SEQ ID NO: 34,
   a sequence having at least 80%, 85% or 90%, preferably at least 91%, 92%, 93%, 94%, 95%, 96%, 97%, 98% or 99% sequence identity to the sequence set forth in SEQ ID NO: 34, or
   an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 34, and
   a light chain variable region comprising or consisting of:
      the amino acid sequence set forth in SEQ ID NO: 36,
      a sequence having at least 80%, 85% or 90%, preferably at least 91%, 92%, 93%, 94%, 95%, 96%, 97%, 98% or 99% sequence identity to the sequence set forth in SEQ ID NO: 36, or
      an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 36;
(7) a heavy chain variable region comprising or consisting of:
   the amino acid sequence set forth in SEQ ID NO: 38,
   a sequence having at least 80%, 85% or 90%, preferably at least 91%, 92%, 93%, 94%, 95%, 96%, 97%, 98% or 99% sequence identity to the sequence set forth in SEQ ID NO: 38, or
   an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 38, and a light chain variable region comprising or consisting of:
      the amino acid sequence set forth in SEQ ID NO: 40,
      a sequence having at least 80%, 85% or 90%, preferably at least 91%, 92%, 93%, 94%, 95%, 96%, 97%, 98% or 99% sequence identity to the sequence set forth in SEQ ID NO: 40, or
      an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 40;
(8) a heavy chain variable region comprising or consisting of:
   the amino acid sequence set forth in SEQ ID NO: 42,
   a sequence having at least 80%, 85% or 90%, preferably at least 91%, 92%, 93%, 94%, 95%, 96%, 97%, 98% or 99% sequence identity to the sequence set forth in SEQ ID NO: 42, or
   an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 42, and a light chain variable region comprising or consisting of:
      the amino acid sequence set forth in SEQ ID NO: 44,
      a sequence having at least 80%, 85% or 90%, preferably at least 91%, 92%, 93%, 94%, 95%, 96%, 97%, 98% or 99% sequence identity to the sequence set forth in SEQ ID NO: 44, or
      an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 44;
(9) a heavy chain variable region comprising or consisting of:
   the amino acid sequence set forth in SEQ ID NO: 46,
   a sequence having at least 80%, 85% or 90%, preferably at least 91%, 92%, 93%, 94%, 95%, 96%, 97%, 98% or 99% sequence identity to the sequence set forth in SEQ ID NO: 46, or
   an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 46, and a light chain variable region comprising or consisting of:
      the amino acid sequence set forth in SEQ ID NO: 48,
      a sequence having at least 80%, 85% or 90%, preferably at least 91%, 92%, 93%, 94%, 95%, 96%, 97%, 98% or 99% sequence identity to the sequence set forth in SEQ ID NO: 48, or
      an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 48;
(10) a heavy chain variable region comprising or consisting of:
   the amino acid sequence set forth in SEQ ID NO: 42,
   a sequence having at least 80%, 85% or 90%, preferably at least 91%, 92%, 93%, 94%, 95%, 96%, 97%, 98% or 99% sequence identity to the sequence set forth in SEQ ID NO: 42, or
   an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 42, and a light chain variable region comprising or consisting of:
      the amino acid sequence set forth in SEQ ID NO: 48,
      a sequence having at least 80%, 85% or 90%, preferably at least 91%, 92%, 93%, 94%, 95%, 96%, 97%, 98% or 99% sequence identity to the sequence set forth in SEQ ID NO: 48, or
      an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 48;
(11) a heavy chain variable region comprising or consisting of:
   the amino acid sequence set forth in SEQ ID NO: 46,
   a sequence having at least 80%, 85% or 90%, preferably at least 91%, 92%, 93%, 94%, 95%, 96%, 97%, 98% or 99% sequence identity to the sequence set forth in SEQ ID NO: 46, or
   an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 46, and
   a light chain variable region comprising or consisting of:
      a sequence having at least 80%, 85% or 90%, preferably at least 91%, 92%, 93%, 94%, 95%, 96%, 97%, 98% or 99% sequence identity to the sequence set forth in SEQ ID NO: 44, or
      an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 44;
(12) a heavy chain variable region comprising or consisting of:
   the amino acid sequence set forth in SEQ ID NO: 34,
   a sequence having at least 80%, 85% or 90%, preferably at least 91%, 92%, 93%, 94%, 95%, 96%, 97%, 98% or 99% sequence identity to the sequence set forth in SEQ ID NO: 34, or
   an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 34, and a light chain variable region comprising or consisting of:
      the amino acid sequence set forth in SEQ ID NO: 28,
      a sequence having at least 80%, 85% or 90%, preferably at least 91%, 92%, 93%, 94%, 95%, 96%, 97%, 98% or 99% sequence identity to the sequence set forth in SEQ ID NO: 28, or
   an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 28; or (13) a heavy chain variable region comprising or consisting of:
      the amino acid sequence set forth in SEQ ID NO: 127,
      a sequence having at least 80%, 85% or 90%, preferably at least 91%, 92%, 93%, 94%, 95%, 96%, 97%, 98% or 99% sequence identity to the sequence set forth in SEQ ID NO: 127, or
   an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 127, and a light chain variable region comprising or consisting of:
      the amino acid sequence set forth in SEQ ID NO: 129,
      a sequence having at least 80%, 85% or 90%, preferably at least 91%, 92%, 93%, 94%, 95%, 96%, 97%, 98% or 99% sequence identity to the sequence set forth in SEQ ID NO: 129, or
      an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 129.

The amino acid sequences of the CDR regions of the antibodies in (1) to (13) above are analyzed by technical means well known to those skilled in the art, for example, by a VBASE2 database.

The antibodies 13E5, 13E5 H1L1, 13E5 H2L2, 13E5 H3L3, 13E5 H4L4 and 13E5 H4L2 disclosed herein share the same HCDR1-3 and LCDR1-3.

According to the IMGT numbering system, the amino acid sequences of the 3 HCDR regions of the heavy chain variable region are as follows:

HCDR1: GYTFTEYT SEQ ID NO: 9

HCDR2: INPNNGGT SEQ ID NO: 10

HCDR3: ARVRRGMDY SEQ ID NO: 11

The amino acid sequences of the 3 CDR regions of the light chain variable region are as follows:

LCDR1: QDVTTA SEQ ID NO: 12

LCDR2: SAS SEQ ID NO: 13

LCDR3: QQHYSAPWT SEQ ID NO: 14

The antibodies 18H7, 18H7 H1L1, 18H7 H2L2, 18H7 H2L3, and 18H7 H3L2 disclosed herein share the same HCDR1-3 and LCDR1-3.

According to the IMGT numbering system, the amino acid sequences of the 3 HCDR regions of the heavy chain variable region are as follows:

HCDR1: GFTFSSSY SEQ ID NO: 15

HCDR2: INSNGGKT SEQ ID NO: 16

HCDR3: TRQRGNYVGAMDY SEQ ID NO: 17

The amino acid sequences of the 3 CDR regions of the light chain variable region are as follows:

LCDR1: QDVSTA SEQ ID NO: 18

CDR2: SAS SEQ ID NO: 19

LCDR3: HQYYGSPPT SEQ ID NO: 20

The antibody 20G10 H3L3 disclosed herein has the following HCDR1-3 and LCDR1-3.

According to the IMGT numbering system, the amino acid sequences of the 3 HCDR regions of the heavy chain variable region are as follows:

HCDR1: GFSLSTSGMG SEQ ID NO: 130

HCDR2: IWWADDK SEQ ID NO: 131

HCDR3: ARITRGNSAMDF SEQ ID NO: 132

The amino acid sequences of the 3 CDR regions of the light chain variable region are as follows:

LCDR1: ENVYSY SEQ ID NO: 133

LCDR2: NAK SEQ ID NO: 134

LCDR3: QHHYGIPWT SEQ ID NO: 135

In one embodiment of the present invention, the antibody further comprises framework regions (FRs) in the heavy chain variable region, preferably the FRs including FR-H1, FR-H2, FR-H3 and FR-H4, wherein the FR-H1 comprises or consists of the amino acid sequence set forth in SEQ ID NO: 49, a sequence having at least 80%, 85% or 90%, preferably at least 91%, 92%, 93%, 94%, 95%, 96%, 97%, 98% or 99% sequence identity to the sequence set forth in SEQ ID NO: 49, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 49; the FR-H2 comprises or consists of the amino acid sequence set forth in SEQ ID NO: 50, a sequence having at least 80%, 85% or 90%, preferably at least 91%, 92%, 93%, 94%, 95%, 96%, 97%, 98% or 99% sequence identity to the sequence set forth in SEQ ID NO: 50, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 50; the FR-H3 comprises or consists of the amino acid sequence set forth in SEQ ID NO: 51, a sequence having at least 80%, 85% or 90%, preferably at least 91%, 92%, 93%, 94%, 95%, 96%, 97%, 98% or 99% sequence identity to the sequence set forth in SEQ ID NO: 51, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 51; and the FR-H4 comprises or consists of the amino acid sequence set forth in SEQ ID NO: 52, a sequence having at least 80%, 85% or 90%, preferably at least 91%, 92%, 93%, 94%, 95%, 96%, 97%, 98% or 99% sequence identity to the sequence set forth in SEQ ID NO: 52, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 52.

In one embodiment of the present invention, the antibody further comprises FRs in the light chain variable region, preferably the FRs including FR-L1, FR-L2, FR-L3 and FR-L4, wherein the FR-L1 comprises or consists of the amino acid sequence set forth in SEQ ID NO: 53, a sequence having at least 80%, 85% or 90%, preferably at least 91%, 92%, 93%, 94%, 95%, 96%, 97%, 98% or 99% sequence identity to the sequence set forth in SEQ ID NO: 53, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 53; the FR-L2 comprises or consists of the amino acid sequence set forth in SEQ ID NO: 54, a sequence having at least 80%, 85% or 90%, preferably at least 91%, 92%, 93%, 94%, 95%, 96%, 97%, 98% or 99% sequence identity to the sequence set forth in SEQ ID NO: 54, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 54; the FR-L3 comprises or consists of the amino acid sequence set forth in SEQ ID NO: 55, a sequence having at least 80%, 85% or 90%, preferably at least 91%, 92%, 93%, 94%, 95%, 96%, 97%, 98% or 99% sequence identity to the sequence set forth in SEQ ID NO: 55, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 55; and the FR-L4 comprises or consists of the amino acid sequence set forth in SEQ ID NO: 56, a sequence having at least 80%, 85% or 90%, preferably at least 91%, 92%, 93%, 94%, 95%, 96%, 97%, 98% or 99% sequence identity to the sequence set forth in SEQ ID NO: 56, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 56.

In one embodiment of the present invention, the antibody further comprises FRs in the heavy chain variable region, preferably the FRs including FR-H1, FR-H2, FR-H3 and FR-H4, wherein the FR-H1 comprises or consists of the amino acid sequence set forth in SEQ ID NO: 57, a sequence having at least 80%, 85% or 90%, preferably at least 91%, 92%, 93%, 94%, 95%, 96%, 97%, 98% or 99% sequence identity to the sequence set forth in SEQ ID NO: 57, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 57; the FR-H2 comprises or consists of the amino acid sequence set forth in SEQ ID NO: 58, a sequence having at least 80%, 85% or 90%, preferably at least 91%, 92%, 93%, 94%, 95%, 96%, 97%, 98% or 99% sequence identity to the sequence set forth in SEQ ID NO: 58, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 58; the FR-H3 comprises or consists of the amino acid sequence set forth in SEQ ID NO: 59, a sequence having at least 80%, 85% or 90%, preferably at least 91%, 92%, 93%, 94%, 95%, 96%, 97%, 98% or 99% sequence identity to the sequence set forth in SEQ ID NO: 59, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 59; and the FR-H4 comprises or consists of the amino acid sequence set forth in SEQ ID NO: 60, a sequence having at least 80%, 85% or 90%, preferably at least 91%, 92%, 93%, 94%, 95%, 96%, 97%, 98% or 99% sequence identity to the sequence set forth in SEQ ID NO: 60, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 60.

In one embodiment of the present invention, the antibody further comprises FRs in the light chain variable region, preferably the FRs including FR-L1, FR-L2, FR-L3 and FR-L4, wherein the FR-L1 comprises or consists of the amino acid sequence set forth in SEQ ID NO: 61, a sequence having at least 80%, 85% or 90%, preferably at least 91%, 92%, 93%, 94%, 95%, 96%, 97%, 98% or 99% sequence identity to the sequence set forth in SEQ ID NO: 61, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 61; the FR-L2 comprises or consists of the amino acid sequence set forth in SEQ ID NO: 62, a sequence having at least 80%, 85% or 90%, preferably at least 91%, 92%, 93%, 94%, 95%, 96%, 97%, 98% or 99% sequence identity to the sequence set forth in SEQ ID NO: 62, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 62; the FR-L3 comprises or consists of the amino acid sequence set forth in SEQ ID NO: 63, a sequence having at least 80%, 85% or 90%, preferably at least 91%, 92%, 93%, 94%, 95%, 96%, 97%, 98% or 99% sequence identity to the sequence set forth in SEQ ID NO: 63, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 63; and the FR-L4 comprises or consists of the amino acid sequence set forth in SEQ ID NO: 64, a sequence having at least 80%, 85% or 90%, preferably at least 91%, 92%, 93%, 94%, 95%, 96%, 97%, 98% or 99% sequence identity to the sequence set forth in SEQ ID NO: 64, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 64.

In one embodiment of the present invention, the antibody further comprises FRs in the heavy chain variable region, preferably the FRs including FR-H1, FR-H2, FR-H3 and FR-H4, wherein the FR-H1 comprises or consists of the amino acid sequence set forth in SEQ ID NO: 65, a sequence having at least 80%, 85% or 90%, preferably at least 91%, 92%, 93%, 94%, 95%, 96%, 97%, 98% or 99% sequence identity to the sequence set forth in SEQ ID NO: 65, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 65; the FR-H2 comprises or consists of the amino acid sequence set forth in SEQ ID NO: 66, a sequence having at least 80%, 85% or 90%, preferably at least 91%, 92%, 93%, 94%, 95%, 96%, 97%, 98% or 99% sequence identity to the sequence set forth in SEQ ID NO: 66, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 66; the FR-H3 comprises or consists of the amino acid sequence set forth in SEQ ID NO: 67, a sequence having at least 80%, 85% or 90%, preferably at least 91%, 92%, 93%, 94%, 95%, 96%, 97%, 98% or 99% sequence identity to the sequence set forth in SEQ ID NO: 67, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 67; and the FR-H4 comprises or consists of the amino acid sequence set forth in SEQ ID NO: 68, a sequence having at least 80%, 85% or 90%, preferably at least 91%, 92%, 93%, 94%, 95%, 96%, 97%, 98% or 99% sequence identity to the sequence set forth in SEQ ID NO: 68, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 68.

In one embodiment of the present invention, the antibody further comprises FRs in the light chain variable region, preferably the FRs including FR-L1, FR-L2, FR-L3 and FR-L4, wherein the FR-L1 comprises or consists of the amino acid sequence set forth in SEQ ID NO: 69, a sequence having at least 80%, 85% or 90%, preferably at least 91%, 92%, 93%, 94%, 95%, 96%, 97%, 98% or 99% sequence identity to the sequence set forth in SEQ ID NO: 69, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 69; the FR-L2 comprises or consists of the amino acid sequence set forth in SEQ ID NO: 70, a sequence having at least 80%, 85% or 90%, preferably at least 91%, 92%, 93%, 94%, 95%, 96%, 97%, 98% or 99% sequence identity to the sequence set forth in SEQ ID NO: 70, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 70; the FR-L3 comprises or consists of the amino acid sequence set forth in SEQ ID NO: 71, a sequence having at least 80%, 85% or 90%, preferably at least 91%, 92%, 93%, 94%, 95%, 96%, 97%, 98% or 99% sequence identity to the sequence set forth in SEQ ID NO: 71, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 71; and the FR-L4 comprises or consists of the amino acid sequence set forth in SEQ ID NO: 72, a sequence having at least 80%, 85% or 90%, preferably at least 91%, 92%, 93%, 94%, 95%, 96%, 97%, 98% or 99% sequence identity to the sequence set forth in SEQ ID NO: 72, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 72.

In one embodiment of the present invention, the antibody further comprises FRs in the heavy chain variable region, preferably the FRs including FR-H1, FR-H2, FR-H3 and FR-H4, wherein the FR-H1 comprises or consists of the amino acid sequence set forth in SEQ ID NO: 73, a sequence having at least 80%, 85% or 90%, preferably at least 91%, 92%, 93%, 94%, 95%, 96%, 97%, 98% or 99% sequence identity to the sequence set forth in SEQ ID NO: 73, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 73; the FR-H2 comprises or consists of the amino acid sequence set forth in SEQ ID NO: 74, a sequence having at least 80%, 85% or 90%, preferably at least 91%, 92%, 93%, 94%, 95%, 96%, 97%, 98% or 99% sequence identity to the sequence set forth in SEQ ID NO: 74, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 74; the FR-H3 comprises or consists of the amino acid sequence set forth in SEQ ID NO: 75, a sequence having at least 80%, 85% or 90%, preferably at least 91%, 92%, 93%, 94%, 95%, 96%, 97%, 98% or 99% sequence identity to the sequence set forth in SEQ ID NO: 75, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 75; and the FR-H4 comprises or consists of the amino acid sequence set forth in SEQ ID NO: 76, a sequence having at least 80%, 85% or 90%, preferably at least 91%, 92%, 93%, 94%, 95%, 96%, 97%, 98% or 99% sequence identity to the sequence set forth in SEQ ID NO: 76, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 76.

In one embodiment of the present invention, the antibody further comprises FRs in the light chain variable region, preferably the FRs including FR-L1, FR-L2, FR-L3 and FR-L4, wherein the FR-L1 comprises or consists of the amino acid sequence set forth in SEQ ID NO: 77, a sequence having at least 80%, 85% or 90%, preferably at least 91%, 92%, 93%, 94%, 95%, 96%, 97%, 98% or 99% sequence identity to the sequence set forth in SEQ ID NO: 77, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 77; the FR-L2 comprises or consists of the amino acid sequence set forth in SEQ ID NO: 78, a sequence having at least 80%, 85% or 90%, preferably at least 91%, 92%, 93%, 94%, 95%, 96%, 97%, 98% or 99% sequence identity to the sequence set forth in SEQ ID NO: 78, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 78; the FR-L3 comprises or consists of the amino acid sequence set forth in SEQ ID NO: 79, a sequence having at least 80%, 85% or 90%, preferably at least 91%, 92%, 93%, 94%, 95%, 96%, 97%, 98% or 99% sequence identity to the sequence set forth in SEQ ID NO: 79, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 79; and the FR-L4 comprises or consists of the amino acid sequence set forth in SEQ ID NO: 80, a sequence having at least 80%, 85% or 90%, preferably at least 91%, 92%, 93%, 94%, 95%, 96%, 97%, 98% or 99% sequence identity to the sequence set forth in SEQ ID NO: 80, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 80.

In one embodiment of the present invention, the antibody further comprises FRs in the heavy chain variable region, preferably the FRs including FR-H1, FR-H2, FR-H3 and FR-H4, wherein the FR-H1 comprises or consists of the amino acid sequence set forth in SEQ ID NO: 81, a sequence having at least 80%, 85% or 90%, preferably at least 91%, 92%, 93%, 94%, 95%, 96%, 97%, 98% or 99% sequence identity to the sequence set forth in SEQ ID NO: 81, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 81; the FR-H2 comprises or consists of the amino acid sequence set forth in SEQ ID NO: 82, a sequence having at least 80%, 85% or 90%, preferably at least 91%, 92%, 93%, 94%, 95%, 96%, 97%, 98% or 99% sequence identity to the sequence set forth in SEQ ID NO: 82, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 82; the FR-H3 comprises or consists of the amino acid sequence set forth in SEQ ID NO: 83, a sequence having at least 80%, 85% or 90%, preferably at least 91%, 92%, 93%, 94%, 95%, 96%, 97%, 98% or 99% sequence identity to the sequence set forth in SEQ ID NO: 83, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 83; and the FR-H4 comprises or consists of the amino acid sequence set forth in SEQ ID NO: 84, a sequence having at least 80%, 85% or 90%, preferably at least 91%, 92%, 93%, 94%, 95%, 96%, 97%, 98% or 99% sequence identity to the sequence set forth in SEQ ID NO: 84, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 84.

In one embodiment of the present invention, the antibody further comprises FRs in the light chain variable region, preferably the FRs including FR-L1, FR-L2, FR-L3 and FR-L4, wherein the FR-L1 comprises or consists of the amino acid sequence set froth in SEQ ID NO: 85, a sequence having at least 80%, 85% or 90%, preferably at least 91%, 92%, 93%, 94%, 95%, 96%, 97%, 98% or 99% sequence identity to the sequence set forth in SEQ ID NO: 85, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 85; the FR-L2 comprises or consists of the amino acid sequence set forth in SEQ ID NO: 86, a sequence having at least 80%, 85% or 90%, preferably at least 91%, 92%, 93%, 94%, 95%, 96%, 97%, 98% or 99% sequence identity to the sequence set forth in SEQ ID NO: 86, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 86; the FR-L3 comprises or consists of the amino acid sequence set forth in SEQ ID NO: 87, a sequence having at least 80%, 85% or 90%, preferably at least 91%, 92%, 93%, 94%, 95%, 96%, 97%, 98% or 99% sequence identity to the sequence set forth in SEQ ID NO: 87, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 87; and the FR-L4 comprises or consists of the amino acid sequence set forth in SEQ ID NO: 88, a sequence having at least 80%, 85% or 90%, preferably at least 91%, 92%, 93%, 94%, 95%, 96%, 97%, 98% or 99% sequence identity to the sequence set forth in SEQ ID NO: 88, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 88.

In one embodiment of the present invention, the antibody further comprises FRs in the heavy chain variable region, preferably the FRs including FR-H1, FR-H2, FR-H3 and FR-H4, wherein the FR-H1 comprises or consists of the amino acid sequence set forth in SEQ ID NO: 89, a sequence having at least 80%, 85% or 90%, preferably at least 91%, 92%, 93%, 94%, 95%, 96%, 97%, 98% or 99% sequence identity to the sequence set forth in SEQ ID NO: 89, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 89; the FR-H2 comprises or consists of the amino acid sequence set forth in SEQ ID NO: 90, a sequence having at least 80%, 85% or 90%, preferably at least 91%, 92%, 93%, 94%, 95%, 96%, 97%, 98% or 99% sequence identity to the sequence set forth in SEQ ID NO: 90, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 90; the FR-H3 comprises or consists of the amino acid sequence set forth in SEQ ID NO: 91, a sequence having at least 80%, 85% or 90%, preferably at least 91%, 92%, 93%, 94%, 95%, 96%, 97%, 98% or 99% sequence identity to the sequence set forth in SEQ ID NO: 91, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 91; and the FR-H4 comprises or consists of the amino acid sequence set forth in SEQ ID NO: 92, a sequence having at least 80%, 85% or 90%, preferably at least 91%, 92%, 93%, 94%, 95%, 96%, 97%, 98% or 99% sequence identity to the sequence set forth in SEQ ID NO: 92, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 92.

In one embodiment of the present invention, the antibody further comprises FRs in the light chain variable region, preferably the FRs including FR-L1, FR-L2, FR-L3 and FR-L4, wherein the FR-L1 comprises or consists of the amino acid sequence set forth in SEQ ID NO: 93, a sequence having at least 80%, 85% or 90%, preferably at least 91%, 92%, 93%, 94%, 95%, 96%, 97%, 98% or 99% sequence identity to the sequence set forth in SEQ ID NO: 93, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 93; the FR-L2 comprises or consists of the amino acid sequence set forth in SEQ ID NO: 94, a sequence having at least 80%, 85% or 90%, preferably at least 91%, 92%, 93%, 94%, 95%, 96%, 97%, 98% or 99% sequence identity to the sequence set forth in SEQ ID NO: 94, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 94; the FR-L3 comprises or consists of the amino acid sequence set forth in SEQ ID NO: 95, a sequence having at least 80%, 85% or 90%, preferably at least 91%, 92%, 93%, 94%, 95%, 96%, 97%, 98% or 99% sequence identity to the sequence set forth in SEQ ID NO: 95, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 95; and the FR-L4 comprises or consists of the amino acid sequence set forth in SEQ ID NO: 96, a sequence having at least 80%, 85% or 90%, preferably at least 91%, 92%, 93%, 94%, 95%, 96%, 97%, 98% or 99% sequence identity to the sequence set forth in SEQ ID NO: 96, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 96.

In one embodiment of the present invention, the antibody further comprises FRs in the heavy chain variable region, preferably the FRs including FR-H1, FR-H2, FR-H3 and FR-H4, wherein the FR-H1 comprises or consists of the amino acid sequence set forth in SEQ ID NO: 97, a sequence having at least 80%, 85% or 90%, preferably at least 91%, 92%, 93%, 94%, 95%, 96%, 97%, 98% or 99% sequence identity to the sequence set forth in SEQ ID NO: 97, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 97; the FR-H2 comprises or consists of the amino acid sequence set forth in SEQ ID NO: 98, a sequence having at least 80%, 85% or 90%, preferably at least 91%, 92%, 93%, 94%, 95%, 96%, 97%, 98% or 99% sequence identity to the sequence set forth in SEQ ID NO: 98, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 98; the FR-H3 comprises or consists of the amino acid sequence set forth in SEQ ID NO: 99, a sequence having at least 80%, 85% or 90%, preferably at least 91%, 92%, 93%, 94%, 95%, 96%, 97%, 98% or 99% sequence identity to the sequence set forth in SEQ ID NO: 99, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 99; and the FR-H4 comprises or consists of the amino acid sequence set forth in SEQ ID NO: 100, a sequence having at least 80%, 85% or 90%, preferably at least 91%, 92%, 93%, 94%, 95%, 96%, 97%, 98% or 99% sequence identity to the sequence set forth in SEQ ID NO: 100, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 100.

In one embodiment of the present invention, the antibody further comprises FRs in the light chain variable region, preferably the FRs including FR-L1, FR-L2, FR-L3 and FR-L4, wherein the FR-L1 comprises or consists of the amino acid sequence set forth in SEQ ID NO: 101, a sequence having at least 80%, 85% or 90%, preferably at least 91%, 92%, 93%, 94%, 95%, 96%, 97%, 98% or 99% sequence identity to the sequence set forth in SEQ ID NO: 101, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 101; the FR-L2 comprises or consists of the amino acid sequence set forth in SEQ ID NO: 102, a sequence having at least 80%, 85% or 90%, preferably at least 91%, 92%, 93%, 94%, 95%, 96%, 97%, 98% or 99% sequence identity to the sequence set forth in SEQ ID NO: 102, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 102; the FR-L3 comprises or consists of the amino acid sequence set forth in SEQ ID NO: 103, a sequence having at least 80%, 85% or 90%, preferably at least 91%, 92%, 93%, 94%, 95%, 96%, 97%, 98% or 99% sequence identity to the sequence set forth in SEQ ID NO: 103, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 103; and the FR-L4 comprises or consists of the amino acid sequence set forth in SEQ ID NO: 104, a sequence having at least 80%, 85% or 90%, preferably at least 91%, 92%, 93%, 94%, 95%, 96%, 97%, 98% or 99% sequence identity to the sequence set forth in SEQ ID NO: 104, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 104.

In one embodiment of the present invention, the antibody further comprises FRs in the heavy chain variable region, preferably the FRs including FR-H1, FR-H2, FR-H3 and FR-H4, wherein the FR-H1 comprises or consists of the amino acid sequence set forth in SEQ ID NO: 105, a sequence having at least 80%, 85% or 90%, preferably at least 91%, 92%, 93%, 94%, 95%, 96%, 97%, 98% or 99% sequence identity to the sequence set forth in SEQ ID NO: 105, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 105; the FR-H2 comprises or consists of the amino acid sequence set forth in SEQ ID NO: 106, a sequence having at least 80%, 85% or 90%, preferably at least 91%, 92%, 93%, 94%, 95%, 96%, 97%, 98% or 99% sequence identity to the sequence set forth in SEQ ID NO: 106, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 106; the FR-H3 comprises or consists of the amino acid sequence set forth in SEQ ID NO: 107, a sequence having at least 80%, 85% or 90%, preferably at least 91%, 92%, 93%, 94%, 95%, 96%, 97%, 98% or 99% sequence identity to the sequence set forth in SEQ ID NO: 107, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 107; and the FR-H4 comprises or consists of the amino acid sequence set forth in SEQ ID NO: 108, a sequence having at least 80%, 85% or 90%, preferably at least 91%, 92%, 93%, 94%, 95%, 96%, 97%, 98% or 99% sequence identity to the sequence set forth in SEQ ID NO: 108, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 108.

In one embodiment of the present invention, the antibody further comprises FRs in the light chain variable region, preferably the FRs including FR-L1, FR-L2, FR-L3 and FR-L4, wherein the FR-L1 comprises or consists of the amino acid sequence set forth in SEQ ID NO: 109, a sequence having at least 80%, 85% or 90%, preferably at least 91%, 92%, 93%, 94%, 95%, 96%, 97%, 98% or 99% sequence identity to the sequence set forth in SEQ ID NO: 109, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 109; the FR-L2 comprises or consists of the amino acid sequence set forth in SEQ ID NO: 110, a sequence having at least 80%, 85% or 90%, preferably at least 91%, 92%, 93%, 94%, 95%, 96%, 97%, 98% or 99% sequence identity to the sequence set forth in SEQ ID NO: 110, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 110; the FR-L3 comprises or consists of the amino acid sequence set forth in SEQ ID NO: 111, a sequence having at least 80%, 85% or 90%, preferably at least 91%, 92%, 93%, 94%, 95%, 96%, 97%, 98% or 99% sequence identity to the sequence set forth in SEQ ID NO: 111, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 111; and the FR-L4 comprises or consists of the amino acid sequence set forth in SEQ ID NO: 112, a sequence having at least 80%, 85% or 90%, preferably at least 91%, 92%, 93%, 94%, 95%, 96%, 97%, 98% or 99% sequence identity to the sequence set forth in SEQ ID NO: 112, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 112.

In one embodiment of the present invention, the antibody further comprises FRs in the heavy chain variable region, preferably the FRs including FR-H1, FR-H2, FR-H3 and FR-H4, wherein the FR-H1 comprises or consists of the amino acid sequence set forth in SEQ ID NO: 113, a sequence having at least 80%, 85% or 90%, preferably at least 91%, 92%, 93%, 94%, 95%, 96%, 97%, 98% or 99% sequence identity to the sequence set forth in SEQ ID NO: 113, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 113; the FR-H2 comprises or consists of the amino acid sequence set forth in SEQ ID NO: 114, a sequence having at least 80%, 85% or 90%, preferably at least 91%, 92%, 93%, 94%, 95%, 96%, 97%, 98% or 99% sequence identity to the sequence set forth in SEQ ID NO: 114, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 114; the FR-H3 comprises or consists of the amino acid sequence set forth in SEQ ID NO: 115, a sequence having at least 80%, 85% or 90%, preferably at least 91%, 92%, 93%, 94%, 95%, 96%, 97%, 98% or 99% sequence identity to the sequence set forth in SEQ ID NO: 115, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 115; and the FR-H4 comprises or consists of the amino acid sequence set forth in SEQ ID NO: 116, a sequence having at least 80%, 85% or 90%, preferably at least 91%, 92%, 93%, 94%, 95%, 96%, 97%, 98% or 99% sequence identity to the sequence set forth in SEQ ID NO: 116, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 116.

In one embodiment of the present invention, the antibody further comprises FRs in the light chain variable region, preferably the FRs including FR-L1, FR-L2, FR-L3 and FR-L4, wherein the FR-L1 comprises or consists of the amino acid sequence set forth in SEQ ID NO: 117, a sequence having at least 80%, 85% or 90%, preferably at least 91%, 92%, 93%, 94%, 95%, 96%, 97%, 98% or 99% sequence identity to the sequence set forth in SEQ ID NO: 117, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 117; the FR-L2 comprises or consists of the amino acid sequence set forth in SEQ ID NO: 118, a sequence having at least 80%, 85% or 90%, preferably at least 91%, 92%, 93%, 94%, 95%, 96%, 97%, 98% or 99% sequence identity to the sequence set forth in SEQ ID NO: 118, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 118; the FR-L3 comprises or consists of the amino acid sequence set forth in SEQ ID NO: 119, a sequence having at least 80%, 85% or 90%, preferably at least 91%, 92%, 93%, 94%, 95%, 96%, 97%, 98% or 99% sequence identity to the sequence set forth in SEQ ID NO: 119, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 119; and the FR-L4 comprises or consists of the amino acid sequence set forth in SEQ ID NO: 120, a sequence having at least 80%, 85% or 90%, preferably at least 91%, 92%, 93%, 94%, 95%, 96%, 97%, 98% or 99% sequence identity to the sequence set forth in SEQ ID NO: 120, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 120.

In one embodiment of the present invention, the antibody further comprises FRs in the heavy chain variable region, preferably the FRs including FR-H1, FR-H2, FR-H3 and FR-H4, wherein the FR-H1 comprises or consists of the amino acid sequence set forth in SEQ ID NO: 136, a sequence having at least 80%, 85% or 90%, preferably at least 91%, 92%, 93%, 94%, 95%, 96%, 97%, 98% or 99% sequence identity to the sequence set forth in SEQ ID NO: 136, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 136; the FR-H2 comprises or consists of the amino acid sequence set forth in SEQ ID NO: 137, a sequence having at least 80%, 85% or 90%, preferably at least 91%, 92%, 93%, 94%, 95%, 96%, 97%, 98% or 99% sequence identity to the sequence set forth in SEQ ID NO: 137, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 137; the FR-H3 comprises or consists of the amino acid sequence set forth in SEQ ID NO: 138, a sequence having at least 80%, 85% or 90%, preferably at least 91%, 92%, 93%, 94%, 95%, 96%, 97%, 98% or 99% sequence identity to the sequence set forth in SEQ ID NO: 138, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 138; and the FR-H4 comprises or consists of the amino acid sequence set forth in SEQ ID NO: 139, a sequence having at least 80%, 85% or 90%, preferably at least 91%, 92%, 93%, 94%, 95%, 96%, 97%, 98% or 99% sequence identity to the sequence set forth in SEQ ID NO: 139, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 139.

In one embodiment of the present invention, the antibody further comprises FRs in the light chain variable region, preferably the FRs including FR-L1, FR-L2, FR-L3 and FR-L4, wherein the FR-L1 comprises or consists of the amino acid sequence set forth in SEQ ID NO: 140, a sequence having at least 80%, 85% or 90%, preferably at least 91%, 92%, 93%, 94%, 95%, 96%, 97%, 98% or 99% sequence identity to the sequence set forth in SEQ ID NO: 140, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 140; the FR-L2 comprises or consists of the amino acid sequence set forth in SEQ ID NO: 141, a sequence having at least 80%, 85% or 90%, preferably at least 91%, 92%, 93%, 94%, 95%, 96%, 97%, 98% or 99% sequence identity to the sequence set forth in SEQ ID NO: 141, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 141; the FR-L3 comprises or consists of the amino acid sequence set forth in SEQ ID NO: 142, a sequence having at least 80%, 85% or 90%, preferably at least 91%, 92%, 93%, 94%, 95%, 96%, 97%, 98% or 99% sequence identity to the sequence set forth in SEQ ID NO: 142, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 142; and the FR-L4 comprises or consists of the amino acid sequence set forth in SEQ ID NO: 143, a sequence having at least 80%, 85% or 90%, preferably at least 91%, 92%, 93%, 94%, 95%, 96%, 97%, 98% or 99% sequence identity to the sequence set forth in SEQ ID NO: 143, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 143.

One aspect of the present invention relates to an isolated polypeptide comprising the sequences set forth in SEQ ID NOs: 9, 10 and 11, wherein the polypeptide specifically binds to human IL-4RA as part of an anti-human IL-4RA antibody, the antibody further comprising the sequences set forth in SEQ ID NOs: 12, 13 and 14.

One aspect of the present invention relates to an isolated polypeptide comprising the sequences set forth in SEQ ID NOs: 15, 16 and 17, wherein the polypeptide specifically binds to human IL-4RA as part of an anti-human IL-4RA antibody, the antibody further comprising the sequences set forth in SEQ ID NOs: 18, 19 and 20.

One aspect of the present invention relates to an isolated polypeptide comprising the sequences set forth in SEQ ID NOs: 12, 13 and 14, wherein the polypeptide specifically binds to human IL-4RA as part of an anti-human IL-4RA antibody, the antibody further comprising the sequences set forth in SEQ ID NOs: 9, 10 and 11.

One aspect of the present invention relates to an isolated polypeptide comprising the sequences set forth in SEQ ID NOs: 18, 19 and 20, wherein the polypeptide specifically binds to human IL-4RA as part of an anti-human IL-4RA antibody, the antibody further comprising the sequences set forth in SEQ ID NOs: 15, 16 and 17.

One aspect of the present invention relates to an isolated polypeptide comprising the sequences set forth in SEQ ID NOs: 130, 131 and 132, wherein the polypeptide specifically binds to human IL-4RA as part of an anti-human IL-4RA antibody, the antibody further comprising the sequences set forth in SEQ ID NOs: 133, 134 and 135.

One aspect of the present invention relates to an isolated polypeptide comprising the sequences set forth in SEQ ID NOs: 133, 134 and 135, wherein the polypeptide specifically binds to human IL-4RA as part of an anti-human IL-4RA antibody, the antibody further comprising the sequences set forth in SEQ ID NOs: 130, 131 and 132.

One aspect of the present invention relates to an isolated polypeptide comprising a sequence selected from the sequences set forth in SEQ ID NOs:
2, 6, 22, 26, 30, 34 and 38, a sequence having at least 80%, 85% or 90%, preferably at least 91%, 92%, 93%, 94%, 95%, 96%, 97%, 98% or 99% sequence identity to the sequences, or an amino acid sequence having one or more (preferably 1, 2, 3, 4, 5, 6, 7, 8, 9 or 10) conservative amino acid mutations (preferably substitutions, insertions or deletions) compared to the sequences, wherein the polypeptide specifically binds to human IL-4RA as part of an anti-human IL-4RA antibody, the antibody further comprising a sequence selected from the sequences set forth in SEQ ID NOs: 4, 8, 24, 28, 32, 36 and 40, a sequence having at least 80%, 85% or 90%, preferably at least 91%, 92%, 93%, 94%, 95%, 96%, 97%, 98% or 99% sequence identity to the sequences, or an amino acid sequence having one or more (preferably 1, 2, 3, 4, 5, 6, 7, 8, 9 or 10) conservative amino acid mutations (preferably substitutions, insertions or deletions) compared to the sequences;
an isolated polypeptide comprising a sequence selected from the sequence set forth in SEQ ID NO: 42, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 44, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence;
an isolated polypeptide comprising a sequence selected from the sequence set forth in SEQ ID NO: 42, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 48, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence;
an isolated polypeptide comprising a sequence selected from the sequence set forth in SEQ ID NO: 46, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 48, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence; or
an isolated polypeptide comprising a sequence selected from the sequence set forth in SEQ ID NO: 34, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 28, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence.

One aspect of the present invention relates to an isolated polypeptide comprising a sequence selected from the sequences set forth in SEQ ID NOs:
4, 8, 24, 28, 32, 36 and 40, a sequence having at least 80%, 85% or 90%, preferably at least 91%, 92%, 93%, 94%, 95%, 96%, 97%, 98% or 99% sequence identity to the sequences, or an amino acid sequence having one or more (preferably 1, 2, 3, 4, 5, 6, 7, 8, 9 or 10) conservative amino acid mutations (preferably substitutions, insertions or deletions) compared to the sequences, wherein the polypeptide specifically binds to human IL-4RA as part of an anti-human IL-4RA antibody, the antibody further comprising a sequence respectively selected from the sequences set forth in SEQ ID NOs: 2, 6, 22, 26, 30, 34 and 38, a sequence having at least 80%, 85% or 90%, preferably at least 91%, 92%, 93%, 94%, 95%, 96%, 97%, 98% or 99% sequence identity to the sequences, or an amino acid sequence having one or more (preferably 1, 2, 3, 4, 5, 6, 7, 8, 9 or 10) conservative amino acid mutations (preferably substitutions, insertions or deletions) compared to the sequences;
an isolated polypeptide comprising a sequence selected from the sequence set forth in SEQ ID NO: 44, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 42, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence;
an isolated polypeptide comprising a sequence selected from the sequence set forth in SEQ ID NO: 48, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 42, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence;
an isolated polypeptide comprising a sequence selected from the sequence set forth in SEQ ID NO: 48, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 46, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence;
an isolated polypeptide comprising a sequence selected from the sequence set forth in SEQ ID NO: 28, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 34, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence;
an isolated polypeptide comprising a sequence selected from the sequence set forth in SEQ ID NO: 127, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 129, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence; or
an isolated polypeptide comprising a sequence selected from the sequence set forth in SEQ ID NO: 129, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 127, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence.

In one embodiment of the present invention, the antigen-binding fragment is selected from Fab, Fab′, F(ab′)₂, Fd, Fv, dAb, Fab/c, complementarity determining region (CDR) fragment, single chain antibody (e.g., scFv), bivalent antibody and domain antibody.

In one embodiment of the present invention, the antibody is a humanized antibody, a chimeric antibody or a multispecific antibody (e.g., bispecific antibody).

In one embodiment of the present invention, the antibody binds to human IL-4RA protein with a K_{D} less than about 10⁻⁵ M, e.g., less than about 10⁻⁶ M, 10⁻⁷ M, 10⁻⁸ M, 10⁻⁹ M or 10⁻¹⁰ M or less. Preferably, the K_{D} is measured by a Fortebio molecular interaction instrument.

In one embodiment of the present invention, the antibody binds to human IL-4RA protein with an EC₅₀ less than about 100 nM, e.g., less than about 10 nM, 1 nM, 0.9 nM, 0.8 nM, 0.7 nM, 0.6 nM, 0.5 nM, 0.4 nM, 0.3 nM, 0.2 nM, 0.1 nM or less. Specifically, the EC₅₀ is measured by indirect ELISA.

In one embodiment of the present invention, the antibody comprises a constant region, and the constant region is derived from a species other than murine, e.g., from a human antibody, preferably from a human IgG, more preferably from IgG4.

In one embodiment of the present invention, the constant region of the antibody is humanized, e.g., the heavy chain constant regions are Ig gamma-4 chain C regions, preferably the Ig gamma-4 chain C region of GenBank ACCESSION No: P01861.1; the light chain constant regions are Ig kappa chain C regions, preferably the Ig kappa chain C region of GenBank ACCESSION No: P01834.

Another aspect of the present invention relates to an isolated polynucleotide encoding a polypeptide comprising the sequences set forth in SEQ ID NOs: 9, 10 and 11, wherein the polypeptide specifically binds to human IL-4RA as part of an anti-human IL-4RA antibody, the antibody further comprising the sequences set forth in SEQ ID NOs: 12, 13 and 14.

One aspect of the present invention relates to an isolated polynucleotide encoding a polypeptide comprising the sequences set forth in SEQ ID NOs: 15, 16 and 17, wherein the polypeptide specifically binds to human IL-4RA as part of an anti-human IL-4RA antibody, the antibody further comprising the sequences set forth in SEQ ID NOs: 18, 19 and 20.

One aspect of the present invention relates to an isolated polynucleotide encoding a polypeptide comprising the sequences set forth in SEQ ID NOs: 12, 13 and 14, wherein the polypeptide specifically binds to human IL-4RA as part of an anti-human IL-4RA antibody, the antibody further comprising the sequences set forth in SEQ ID NOs: 9, 10 and 11.

One aspect of the present invention relates to an isolated polynucleotide encoding a polypeptide comprising the sequences set forth in SEQ ID NOs: 18, 19 and 20, wherein the polypeptide specifically binds to human IL-4RA as part of an anti-human IL-4RA antibody, the antibody further comprising the sequences set forth in SEQ ID NOs: 15, 16 and 17.

One aspect of the present invention relates to an isolated polynucleotide encoding a polypeptide comprising the sequences set forth in SEQ ID NOs: 130, 131 and 132, wherein the polypeptide specifically binds to human IL-4RA as part of an anti-human IL-4RA antibody, the antibody further comprising the sequences set forth in SEQ ID NOs: 133, 134 and 135.

One aspect of the present invention relates to an isolated polynucleotide encoding a polypeptide comprising the sequences set forth in SEQ ID NOs: 133, 134 and 135, wherein the polypeptide specifically binds to human IL-4RA as part of an anti-human IL-4RA antibody, the antibody further comprising the sequences set forth in SEQ ID NOs: 130, 131 and 132.

One aspect of the present invention relates to an isolated polynucleotide encoding a polypeptide comprising a sequence selected from the sequences set forth in SEQ ID NOs: 2, 6, 22, 26, 30, 34 and 38, a sequence having at least 80%, 85% or 90%, preferably at least 91%, 92%, 93%, 94%, 95%, 96%, 97%, 98% or 99% sequence identity to the sequences, or an amino acid sequence having one or more (preferably 1, 2, 3, 4, 5, 6, 7, 8, 9 or 10) conservative amino acid mutations (preferably substitutions, insertions or deletions) compared to the sequences, wherein the polypeptide specifically binds to human IL-4RA as part of an anti-human IL-4RA antibody, the antibody further comprising a sequence respectively selected from the sequences set forth in SEQ ID NOs: 4, 8, 24, 28, 32, 36 and 40, a sequence having at least 80%, 85% or 90%, preferably at least 91%, 92%, 93%, 94%, 95%, 96%, 97%, 98% or 99% sequence identity to the sequences, or an amino acid sequence having one or more (preferably 1, 2, 3, 4, 5, 6, 7, 8, 9 or 10) conservative amino acid mutations (preferably substitutions, insertions or deletions) compared to the sequences;
an isolated polynucleotide encoding a polypeptide comprising a sequence selected from the sequence set forth in SEQ ID NO: 42, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 44, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence;
an isolated polynucleotide encoding a polypeptide comprising a sequence selected from the sequence set forth in SEQ ID NO: 42, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 48, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence;
an isolated polynucleotide encoding a polypeptide comprising a sequence selected from the sequence set forth in SEQ ID NO: 46, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 48, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence; or
an isolated polynucleotide encoding a polypeptide comprising a sequence selected from the sequence set forth in SEQ ID NO: 34, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 28, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence.

One aspect of the present invention relates to an isolated polynucleotide encoding a polypeptide comprising a sequence selected from the sequences set forth in SEQ ID NOs: 4, 8, 24, 28, 32, 36 and 40, a sequence having at least 80%, 85% or 90%, preferably at least 91%, 92%, 93%, 94%, 95%, 96%, 97%, 98% or 99% sequence identity to the sequences, or an amino acid sequence having one or more (preferably 1, 2, 3, 4, 5, 6, 7, 8, 9 or 10) conservative amino acid mutations (preferably substitutions, insertions or deletions) compared to the sequences, wherein the polypeptide specifically binds to human IL-4RA as part of an anti-human IL-4RA antibody, the antibody further comprising a sequence respectively selected from the sequences set forth in SEQ ID NOs: 2, 6, 22, 26, 30, 34 and 38, a sequence having at least 80%, 85% or 90%, preferably at least 91%, 92%, 93%, 94%, 95%, 96%, 97%, 98% or 99% sequence identity to the sequences, or an amino acid sequence having one or more (preferably 1, 2, 3, 4, 5, 6, 7, 8, 9 or 10) conservative amino acid mutations (preferably substitutions, insertions or deletions) compared to the sequences;
an isolated polynucleotide encoding a polypeptide comprising a sequence selected from the sequence set forth in SEQ ID NO: 44, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 42, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence;
an isolated polynucleotide encoding a polypeptide comprising a sequence selected from the sequence set forth in SEQ ID NO: 48, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 42, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence;
an isolated polynucleotide encoding a polypeptide comprising a sequence selected from the sequence set forth in SEQ ID NO: 48, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 46, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence;
an isolated polynucleotide encoding a polypeptide comprising a sequence selected from the sequence set forth in SEQ ID NO: 28, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 34, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence;
an isolated polynucleotide encoding a polypeptide comprising a sequence selected from the sequence set forth in SEQ ID NO: 127, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 129, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence; or
an isolated polynucleotide encoding a polypeptide comprising a sequence selected from the sequence set forth in SEQ ID NO: 129, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence, wherein the polypeptide specifically binds to human IL-4RA as part of an anti-human IL-4RA antibody, the antibody further comprising a sequence selected from the sequence set forth in SEQ ID NO: 127, a sequence having at least 80%, 85% or 90%, preferably at least 91%, 92%, 93%, 94%, 95%, 96%, 97%, 98% or 99% sequence identity to the sequence, or an amino acid sequence having one or more (preferably 1, 2, 3, 4, 5, 6, 7, 8, 9 or 10) conservative amino acid mutations (preferably substitutions, insertions or deletions) compared to the sequence.

In particular, the polynucleotide molecule comprises or consists of the sequences set forth in SEQ ID NO: 1, SEQ ID NO: 5, SEQ ID NO: 21, SEQ ID NO: 25, SEQ ID NO: 29, SEQ ID NO: 33, SEQ ID NO: 37, SEQ ID NO: 41, SEQ ID NO: 45 or SEQ ID NO: 126, or a sequence having at least 80%, 85% or 90%, preferably at least 91%, 92%, 93%, 94%, 95%, 96%, 97%, 98% or 99% sequence identity to the sequences.

In particular, the polynucleotide molecule comprises or consists of the sequences set forth in SEQ ID NO: 3, SEQ ID NO: 7, SEQ ID NO: 23, SEQ ID NO: 27, SEQ ID NO: 31, SEQ ID NO: 35, SEQ ID NO: 39, SEQ ID NO: 43, SEQ ID NO: 47 or SEQ ID NO: 128, or a sequence having at least 80%, 85% or 90%, preferably at least 91%, 92%, 93%, 94%, 95%, 96%, 97%, 98% or 99% sequence identity to the sequences.

Yet another aspect of the present invention relates to a vector comprising any one of the polynucleotide molecules disclosed herein.

Yet another aspect of the present invention relates to a host cell comprising any one of the polynucleotide molecules disclosed herein, or the vector disclosed herein.

Yet another aspect of the present invention relates to a method for preparing any one of the antibodies or the antigen-binding fragments thereof disclosed herein, comprising culturing the host cell disclosed herein in a suitable condition, and isolating the antibody or the antigen-binding fragment thereof from the cell cultures.

One aspect of the present invention further provides an antibody conjugate comprising the anti-human IL-4RA antibody or the antigen-binding fragment thereof, and a conjugated moiety coupled thereto, wherein the conjugated moiety is a purification tag (e.g., a His tag), a cytotoxic agent or a detectable label. Preferably, the conjugated moiety is a radioisotope, a luminescent substance, a colored substance, an enzyme or polyethylene glycol.

One aspect of the present invention further provides a multispecific antibody, preferably a bispecific antibody, comprising the anti-human IL-4RA antibody or the antigen-binding fragment thereof, and an antibody or an antigen-binding fragment against another antigen and/or another antigenic epitope.

One aspect the present invention further provides a fusion protein comprising any one of the anti-human IL-4RA antibodies or the antigen-binding fragments thereof disclosed herein.

One aspect the present invention further provides a kit comprising any one of the antibodies or the antigen-binding fragments thereof disclosed herein, or the antibody conjugate or the multispecific antibody disclosed herein.

Preferably, the kit further comprises a second antibody that specifically recognizes the antibody or the antigen-binding fragment thereof; optionally, the second antibody further comprises a detectable label, such as a radioisotope, a luminescent substance, a colored substance, an enzyme or polyethylene glycol.

Yet another aspect of the present invention relates to use of any one of the antibodies or the antigen-binding fragments thereof disclosed herein, or the antibody conjugate or the multispecific antibody disclosed herein in preparing a kit for detecting the presence or level of human IL-4RA in a sample or for the prevention and/or treatment and/or adjuvant treatment and/or diagnosis of an allergic disease, a tumor, an autoimmune disease, a skin infection, tissue fibrosis, rhinosinusitis, nasal polyps and chronic obstructive pulmonary disease, wherein preferably the allergic disease is selected from atopic dermatitis, allergic rhinitis, asthma and allergy, and more preferably, atopic dermatitis includes moderate and severe atopic dermatitis.

Yet another aspect of the present invention relates to a pharmaceutical composition comprising any one of the antibodies or the antigen-binding fragments thereof disclosed herein or the antibody conjugate, the multispecific antibody or the fusion protein disclosed herein, and optionally, a pharmaceutically acceptable carrier and/or excipient. Preferably, the pharmaceutical composition is used alone or in combination with one or more medicaments. When the pharmaceutical composition cannot be directly mixed with the combined medicament, the pharmaceutical composition and the combined medicament are separately present in the kit.

Yet another aspect of the present invention relates to use of any one of the antibodies or the antigen binding fragments thereof disclosed herein or the antibody conjugate, the multispecific antibody or the fusion protein disclosed herein in preparing:
a medicament for blocking the binding of human IL-4RA to IL-4 or IL-13,
a medicament for blocking the activity of human IL-4RA or down-regulating the level of human IL-4RA, and
a medicament for blocking a cellular biological response mediated by the binding of human IL-4 or human IL-13 to IL-4RA;
wherein preferably, the ligand of human IL-4RA is human IL-4 or human IL-13, more preferably, human IL-4.

One aspect of the present invention relates to use of any one of the antibodies or the antigen binding fragments thereof disclosed herein or the antibody conjugate, the multispecific antibody or the fusion protein disclosed herein in preparing a medicament for treating a disease selected from: an atopic dermatitis including moderate and severe atopic dermatitis; nasal polyps; asthma; a skin infection; an autoimmune disease, and the like.

Yet another aspect of the present invention relates to an in vivo or in vitro method comprising administering a cell comprising the antibody or the antigen-binding fragment thereof disclosed herein, the antibody conjugate, the multispecific antibody or the fusion protein disclosed herein, or administering to a subject in need an effective amount of any one of the antibodies or the antigen-binding fragments thereof or the antibody conjugate, the multispecific antibody or the fusion protein disclosed herein. The method is selected from:
a method for blocking the binding of IL-4RA to IL-4 or IL-13,
a method for down-regulating IL-4RA activity or level, or
a method for blocking a cellular biological response mediated by the binding of human IL-4 or human IL-13 to IL-4RA;
wherein preferably, the ligand of IL-4RA is IL-4 or IL-13, more preferably, IL-4.

In one embodiment of the present invention, the in vitro method is non-therapeutic and/or non-diagnostic.

Yet another aspect of the present invention relates to use of any one of the antibodies or the antigen-binding fragments thereof or the antibody conjugate or the multispecific antibody disclosed herein in preparing a medicament for the prevention and/or treatment and/or adjuvant treatment and/or diagnosis of an allergic disease, a tumor, a skin infection, an autoimmune disease, tissue fibrosis, rhinosinusitis, nasal polyps and chronic obstructive pulmonary disease. In particular, the allergic disease is selected from atopic dermatitis, allergic rhinitis, asthma and allergy.

In one embodiment of the present invention, the medicament is in a dosage form suitable for oral administration to the gastrointestinal (GI) tract. Preferably the dosage form is selected from tablets, capsules, pills, powders, granules, emulsions, microemulsions, solutions, suspensions, syrups and elixirs.

In one embodiment of the present invention, the medicament is in a form suitable for administration by subcutaneous injection, intradermal injection, intravenous injection, intramuscular injection or intralesional injection.

Yet another aspect of the present invention relates to a method for the prevention and/or treatment and/or adjuvant treatment and/or diagnosis of an allergic disease, a tumor, an autoimmune disease, tissue fibrosis, a skin infection, rhinosinusitis, nasal polyps, chronic obstructive pulmonary disease, comprising administering any of the antibody or the antigen-binding fragment thereof, the antibody conjugate, or the multispecific antibody disclosed herein to a subject in need. In particular, the allergic disease is selected from atopic dermatitis, allergic rhinitis, asthma and allergy.

In the present invention, unless otherwise defined, the scientific and technical terms used herein have the meanings generally understood by those skilled in the art. In addition, the laboratory operations of cell culture, molecular genetics, nucleic acid chemistry and immunology used in the present invention are the routine operations widely used in the corresponding fields. Meanwhile, in order to better understand the present invention, the definitions and explanations of the relevant terms are provided below.

As used herein, the term “antigen-binding fragment” means a protein or portion of a protein that specifically binds to a given antigen. For example, a portion of an antibody comprising amino acid residues that interact with an antigen and confer the antibody the specificity and affinity for the antigen is referred to as an “antigen-binding fragment”. The antigen-binding fragment generally comprises one or more complementary-determining regions (CDRs). Certain antigen-binding fragments further comprise one or more framework regions (FRs). CDRs are amino acid sequences that contribute to antigen binding specificity and affinity.

As used herein, the term “antibody” refers to an intact immunoglobulin of any isotype or an antigen-binding fragment thereof that can compete with an intact antibody for specifically binding to a target antigen, and includes, for example, chimeric, humanized, fully human, and bispecific antibodies or antigen-binding fragments thereof. Such “antibodies” are antigen-binding proteins. An intact antibody generally comprises at least two full-length heavy chains and two full-length light chains, but, in some cases, may comprise fewer chains, such as an antibody naturally occurring in Camelidae species that may comprise only a heavy chain. An antibody or antigen-binding fragment thereof may be derived from a single source only, or may be “chimeric”, i.e., different portions of an antibody may be derived from two different sources as further described below. Antibodies or antigen-binding fragments thereof can be produced in hybridomas by recombinant DNA techniques, or by enzymatic or chemical cleavage of intact antibodies. Unless otherwise indicated, the term “antibody”, in addition to antibodies comprising two full-length heavy chains and two full-length light chains, also includes derivatives, variants and fragments thereof.

As used herein, the term “antigen-binding fragment” (or simply “fragment”) of an “antibody” or “immunoglobulin chain” (heavy or light chain) includes a portion of an antibody (whether obtained or synthesized) that lacks at least some of the amino acid residues present in the full length of the antibody but is capable of specifically binding the antigen. Such fragments are biologically active as they specifically bind to a target antigen and can compete with other antibodies or antigen binding fragments thereof for specifically binding to a given epitope. In one aspect, such fragments will retain at least one CDR present in the full-length light or heavy chain of the antibody, and in some embodiments, will comprise a single heavy and/or light chain or a portion thereof. Such biologically active fragments can be produced by recombinant DNA techniques, or, for example, by enzymatic or chemical cleavage of intact antibodies. Immunologically functional immunoglobulin fragments include, but are not limited to, Fab, Fab′, F(ab′)₂, Fv, domain antibodies, and single-chain antibodies, and can be derived from any mammalian source, including, but not limited to, human, mouse, rat, Camelidae species, and rabbit. It is further contemplated that a functional portion of an antibody disclosed herein, such as one or more CDRs, can be covalently bound to a second protein or a small molecule to generate a therapeutic agent that targets a particular target in the body, thereby having bifunctional therapeutic properties or having an extended serum half-life, such as a fusion protein.

As used herein, the terms “antibody full-length chain”, “full-length antibody”, “intact antibody” and “whole antibody” are used interchangeably herein to refer to such an antibody having a substantially similar structure to a natural antibody structure or having a heavy chain that comprises an Fc region as defined herein.

The term “light chain” includes full-length light chains and fragments thereof having a sufficient variable region sequence to confer the binding specificity. The full-length light chain comprises a variable region domain VL and a constant region domain CL. The variable region domain of the light chain is at the amino terminus of the polypeptide. Light chains include kappa (κ) and lambda (λ) chains.

The term “heavy chain” includes full-length heavy chains and fragments thereof having a sufficient variable region sequence to confer the binding specificity. The full-length heavy chain comprises a variable region domain VH and 3 constant region domains CH1, CH2 and CH3. The VH domain is at the amino terminus of the polypeptide and the CH domains are at the carboxyl terminus, in which CH3 is closest to the carboxyl terminus of the polypeptide. The heavy chain may be of any isotype, including IgG (including IgG1, IgG2, IgG3 and IgG4 subtypes), IgA (including IgA1 and IgA2 subtypes), IgM and IgE.

As used herein, the term “Fab” fragment consists of one light chain, CH1 and the variable region of one heavy chain. The heavy chain of a Fab molecule cannot form disulfide bonds with another heavy chain molecule.

As used herein, the term “Fc” region comprises two heavy chain fragments comprising the CH1 and CH2 domains of an antibody. The two heavy chain fragments are held together by two or more disulfide bonds and the hydrophobic interaction of the CH3 domains.

As used herein, the term “Fab”′ fragment comprises portions of one light chain and one heavy chain including the VH domain and the CH1 domain and the region between the CH1 and CH2 domains) such that interchain disulfide bonds can be formed between the two heavy chains of two Fab′ fragments to give an F(ab′)₂ molecule.

As used herein, the term “F(ab′)₂” fragment comprises two light chains and two heavy chains containing portions of the constant region between the CH1 and CH2 domains such that interchain disulfide bonds are formed between the two heavy chains. Thus the F(ab′)₂ fragment consists of two Fab′ fragments held together by disulfide bonds between the two heavy chains.

As used herein, the term “Fv” region comprises the variable regions from the heavy and light chains, but lacks the constant regions.

As used herein, the term “Fd” fragment refers to an antibody fragment consisting of VH and CH1 domains (Ward et al., Nature, 341:544-546 (1989)).

As used herein, the term “dAb” fragment consists of a VH domain (Ward et al., Nature 341:544-546 (1989)).

As used herein, the term “Fab′-SH″ is the designation herein for Fab′, wherein one or more cysteine residues of the constant domain carry a free thiol group.

As used herein, the term “Fab/c” fragment is an intermediate formed by pepsin digestion of an immunoglobulin, which combines the advantages of Fab and Fc regions, i.e., strong diffusibility and low metabolic clearance in vivo, while retaining high affinity (Liu Jianjun, Chinese Journal of Cellular and Molecular Immunology, 1989(4):29-29).

As used herein, the term “single-chain antibody” is an Fv molecule in which the heavy and light chain variable regions are connected by a flexible linker to form a single polypeptide chain (which forms an antigen binding region) (see, e.g., Bird et al., Science, 242:423-426 (1988), and Huston et al., Proc. Natl. Acad. Sci., USA, 90:5879-5883 (1988)). Single-chain antibodies are described in detail in International Patent Publication No. WO88/01649 and U.S. Pat. Nos. 4,946,778 and 5,260,203, the disclosures of which are incorporated herein by reference.

As used herein, the term “domain antibody” is an immunofunctional immunoglobulin fragment that comprises only the variable region of the heavy chain or the light chain. In some cases, two or more VH regions are covalently linked by a peptide linker to generate a multivalent domain antibody (particularly a bivalent domain antibody). The two VH regions of the bivalent domain antibody may target the same or different antigens.

As used herein, the term “bivalent antigen-binding protein” or “bivalent antibody” comprises two antigen-binding sites. In some cases, the two binding sites have specificity against the same antigen. The bivalent antibody may be bispecific.

As used herein, the term “multispecific antigen-binding protein” or “multispecific antibody” is an antigen-binding protein or antibody that targets more than one antigen or epitope.

As used herein, the term “bispecific”, “dual-specificity” or “bifunctional” antigen-binding protein or antibody is a hybrid antigen-binding protein or antibody having two different antigen-binding sites, respectively. A bispecific antibody is a multispecific antigen-binding protein or multispecific antibody, and can be produced by a variety of methods, including but not limited to, fusion of hybridomas or linkage of Fab′ fragments. See, e.g., Songsivilai and Lachmann, 1990, Clin. Exp. Immunol., 79:315-321; Kostelny et al., 1992, J. Immunol., 148:1547-1553. The two binding sites of a bispecific antigen-binding protein or antibody will bind to two different epitopes that are present in the same or different protein targets.

As used in the present invention, the terms “mAb” and “monoclonal antibody” refer to an antibody or a fragment of an antibody that is derived from a group of highly homologous antibodies, i.e., from a group of identical antibody molecules, except for natural mutations that may occur spontaneously. The monoclonal antibody has a high specificity for a single epitope on an antigen. The polyclonal antibody, relative to the monoclonal antibody, generally comprises at least two or more different antibodies which generally recognize different epitopes on an antigen. Monoclonal antibodies can generally be obtained using hybridoma technique first reported by Kohler et al. (Nature, 256:495, 1975), but can also be obtained using recombinant DNA technique (see, e.g., U.S. Pat. No. 4,816,567).

As used in the present invention, the term “humanized antibody” refers to an antibody or antibody fragment obtained when all or a part of CDR regions of a human immunoglobulin (receptor antibody) are replaced by the CDR regions of a non-human antibody (donor antibody), wherein the donor antibody may be a non-human (e.g., mouse, rat or rabbit) antibody having expected specificity, affinity or reactivity. In addition, some amino acid residues in the framework regions (FRs) of the receptor antibody can also be replaced by the amino acid residues of corresponding non-human antibodies or by the amino acid residues of other antibodies to further improve or optimize the performance of the antibody. For more details on humanized antibodies, see, for example, Jones et al., Nature, 321:522-525 (1986); Reichmann et al., Nature, 332:323-329 (1988); Presta, Curr. Op. Struct. Biol., 2:593-596 (1992); and Clark, Immunol. Today, 21:397-402 (2000).

As used herein, the term “epitope” refers to a site on an antigen at which an immunoglobulin or antibody specifically binds. “Epitope” is also called in the field as an “antigenic determinant”. The epitope or antigenic determinant generally consists of chemically active surface groups of molecules such as amino acids, carbohydrates or sugar side chains, and usually has specific three-dimensional structural characteristics and specific charge characteristics. For example, the epitope generally includes at least 3, 4, 5, 6, 7, 8, 9, 10, 11, 12, 13, 14, or 15 consecutive or non-consecutive amino acids in a unique spatial conformation, which can be “linear” or “conformational”. See, for example, Epitope Mapping Protocols in Methods in Molecular Biology, Vol. 66, G. E. Morris, Ed. (1996). In a linear epitope, all interaction sites between a protein and an interaction molecule (e.g., an antibody) are located adjacently along the primary amino acid sequence of the protein. In a conformational epitope, the interaction sites are located across sections of the amino acid residue.

The terms “polypeptide” or “protein” are used interchangeably herein to refer to a polymer of amino acid residues. The term is also used to refer to an amino acid polymer in which one or more amino acid residues are analogs or mimetics of naturally existing amino acids, and for naturally existing amino acid polymers. The term may also include, for example, amino acid polymers that have been modified by addition of saccharide residues to form glycoproteins, or have been phosphorylated. Polypeptides and proteins can be produced by naturally existing cells and non-recombinant cells; or they may be produced by genetically engineered or recombinant cells, and comprise a molecule having the amino acid sequence of a native protein or a molecule having deletions, insertions and/or substitutions in one or more amino acids of the native sequence.

In particular, the terms “polypeptide” and “protein” include antibodies, such as anti-human IL-4RA antibodies (also referred to as IL-4RA antibodies), IL-4RA-binding proteins, antibodies or sequences having deletions, insertions, and/or substitutions in one or more amino acids of an antigen-binding protein.

The term “polypeptide fragment” refers to a polypeptide having amino-terminal deletions, carboxyl-terminal deletions, and/or internal deletions as compared to a full-length protein. Such fragments may also contain modified amino acids as compared to the full-length protein. In certain embodiments, such fragments are about 5 to 500 amino acids in length. For example, a fragment can be at least 5, 6, 8, 10, 14, 20, 50, 70, 100, 110, 150, 200, 250, 300, 350, 400 or 450 amino acids in length. Useful polypeptide fragments include immunologically functional fragments of antibodies, including binding domains. In the case of human IL-4RA antibodies, useful fragments include but are not limited to, CDR regions, variable domains of heavy or light chains, portions of antibody chains, variable domains just comprising 2 CDRs, or the like.

The terms “human IL-4RA”, “hIL-4RA”, “human IL-4 receptor A”, “human IL-4 receptor alpha subunit” are used interchangeably to refer to human interleukin-4 receptor alpha subunit. Human IL-4RA refers to its mature peptide (Genbank ID: NP_001244336.1). IL-4 and IL-13 are the main endogenous agonists of IL-4RA. Unless otherwise indicated or clear from the context in which the term is used, “IL-4RA” refers to human IL-4RA.

A “derivative” of a polypeptide is a polypeptide (e.g., an antigen-binding protein or antibody) that is chemically modified in other manners than insertion, deletion or substitution, e.g., by conjugation with another chemical moiety, e.g., a PEG-conjugated polypeptide.

As used herein, the term “isolated” refers to “obtained by artificial means from a natural state”. If a certain “isolated” substance or component appears in nature, it may be that change occurs in its natural environment, or that it is isolated from the natural environment, or both. For example, a certain non-isolated polynucleotide or polypeptide naturally exists in a certain living animal, and the same polynucleotide or polypeptide with a high purity isolated from such a natural state is called isolated polynucleotide or polypeptide. The term “isolated” does not exclude the existence of artificial or synthetic substances or other impurities that do not affect the activity of the substance.

As used herein, the term “vector” refers to a nucleic acid vehicle into which a polynucleotide can be inserted. When a vector allows for the expression of the protein encoded by the inserted polynucleotide, the vector is called an expression vector. A vector can be introduced into a host cell by transformation, transduction, or transfection so that the genetic substance elements carried by the vector can be expressed in the host cell. Vectors are well known to those skilled in the art, including but not limited to: plasmids; phagemids; cosmids; artificial chromosomes, such as yeast artificial chromosome (YAC), bacterial artificial chromosome (BAC), or P1-derived artificial chromosome (PAC); phages such as lambda phages or M13 phages; and animal viruses. Animal viruses that can be used as vectors include, but are not limited to retroviruses (including lentiviruses), adenoviruses, adeno-associated viruses, herpes viruses (such as herpes simplex virus), poxviruses, baculoviruses, papillomaviruses, and papovaviruses (such as SV40). A vector can contain a variety of elements that control expression, including, but not limited to promoter sequences, transcription initiation sequences, enhancer sequences, selection elements, and reporter genes. In addition, the vector may further contain a replication initiation site.

As used herein, the term “host cell” refers to cells that can be introduced with vectors, including, but not limited to, prokaryotic cells such as E. coli or bacillus subtilis, fungal cells such as yeast cells or aspergillus, insect cells such as S2 drosophila cells or Sf9, or animal cells such as fibroblasts, CHO cells, COS cells, NSO cells, HeLa cells, BHK cells, HEK 293 cells, or human cells.

As used herein, the term “specifically bind” refers to a non-random binding reaction between two molecules, such as a reaction between an antibody and an antigen it targets. In some embodiments, an antibody that specifically binds to an antigen (or an antibody that is specific for an antigen) refers to that the antibody binds to the antigen with an affinity (K_{D}) of less than about 10⁻⁵ M, such as less than about 10⁻⁶ M, 10⁻⁷ M, 10⁻⁸ M, 10⁻⁹ M or 10⁻¹⁰ M or less.

As used herein, the term “K_{D}” refers to a dissociation equilibrium constant for a specific antibody-antigen interaction, which is used to describe the binding affinity between the antibody and the antigen. Among the several parameters measured by molecular binding kinetics, the K_{D} value is the dissociation equilibrium constant. In an antibody drug research, it is the parameter characterizing the intensity of the affinity effect of an antibody of interest and the target antigen molecule, and is calculated by the formula: K_{D} = k_{dis}/kₒₙ. A smaller equilibrium dissociation constant indicates a more intensive antibody-antigen binding and a higher affinity between the antibody and the antigen. kₒₙ (association rate constant) is the rate of antigen-antibody complex formation, and a smaller kₒₙ suggests a faster binding of an antibody to an antigen. k_{dis} (dissociation rate constant) is the rate at which an antibody dissociates from an antigen-antibody complex, and a smaller k_{dis} suggests a slower rate for the antibody dissociating from the antigen and a firmer binding between the antibody and the antigen. Generally, an antibody binds to an antigen (e.g., L1 protein) with a dissociation equilibrium constant (K_{D}) of less than about 10⁻⁵ M, such as less than about 10⁻⁶ M, 10⁻⁷ M, 10⁻⁸ M, 10⁻⁹ M or 10⁻¹⁰ M or less, for example, as determined on a BIACORE surface plasmon resonance (SPR) instrument or a ForteBio molecular interaction instrument.

As used herein, the terms “monoclonal antibody” and “McAb” have the same meaning and can be used interchangeably; the terms “polyclonal antibody” and “PcAb” have the same meaning and can be used interchangeably; the terms “polypeptide” and “protein” have the same meaning and can be used interchangeably. Besides, herein, amino acids are generally represented by single-letter and three-letter abbreviations known in the art. For example, alanine can be represented by A or Ala.

As used herein, the terms “hybridoma” and “hybridoma cells” can be used interchangeably, and when referring to the terms “hybridoma” and “hybridoma cells”, subclones and progeny cells of the hybridoma are also included.

As used herein, the terms “percent sequence identity” and “percent sequence homology” are used interchangeably.

As used herein, the terms “similarity”, “sequence similarity” and “identity” refers to the correlation of the sequences of two or more protein or polypeptide molecules, as determined by aligning and comparing the sequences. “Percent identity” refers to the percentage of identical amino acid residues in the molecules compared, and can be calculated based on the size of the smallest molecule for comparison. For such calculations, gaps in the alignment (if any) must be addressed by a particular mathematical model or computer program (i.e., an “algorithm”). The term “substantial identity”, when used for polypeptides, refers to that two peptide sequences, when optimally aligned, for example using the programs GAP or BESTFIT, using default gap weights provided by the programs, have at least 70%, 75% or 80% sequence identity, at least 90% or 95% sequence identity, or at least 97%, 98% or 99% sequence identity. In some cases, residue positions that are not identical differ by conservative amino acid substitutions. “Conservative amino acid substitution” is one in which the amino acid residue is replaced with another amino acid residue having a side chain R group that possesses similar chemical properties (e.g., charge or hydrophilicity). Generally, conservative amino acid substitutions will substantially retain the functions and properties of the protein. In cases where two or more amino acid sequences differ from each other by conservative substitutions, the percent sequence identity may be elevated to correct for the conservative nature of the substitution. Methods for making this adjustment are well known to those skilled in the art. See, e.g., Pearson, Methods Mol. Biol., 243:307-31 (1994). Examples of groups of amino acids having side chains with similar chemical properties include: 1) aliphatic hydroxy side chain: glycine, alanine, valine, leucine and isoleucine, 2) aliphatic hydroxy side chain: serine and threonine, 3) amide-containing side chain: asparagine and glutamine, 4) aromatic side chain: phenylalanine, tyrosine and tryptophan, 5) basic side chain: lysine, arginine and histidine, 6) acidic side chain: aspartic acid and glutamic acid, and 7) sulfur-containing side chain: cysteine and methionine. The conservative amino acid substitution groups are valine-leucine-isoleucine, phenylalanine-tyrosine, lysine-arginine, alanine-valine, glutamic acid-aspartic acid and asparagine-glutamine Optionally, a conservative substitution is any change with a positive value in the PAM250 log-likelihood matrix disclosed in Gonnet et al., Science, 256:1443-45 (1992), which is incorporated herein by reference. A “moderately conservative” substitution is any change with a non-negative value in the PAM250 log-likelihood matrix.

Sequence identity of polypeptides is usually measured by sequence analysis software. Protein analysis software matches sequences using a measure of similarity assigned to different substitutions, deletions and other modifications (including conservative amino acid substitutions). For example, GCG, including programs such as “Gap” and “Bestfit” which (using default parameters specified by the program) can be used to determine sequence homology or sequence identity between closely related polypeptides (e.g., homologous polypeptides from different biological species) or between a wild-type protein and its mutant protein. See, for example, GCG Version 6.1 (University of Wisconsin, WI). Polypeptide sequences can also be compared using FASTA with default or recommended parameters. See GCG Version 6.10 FASTA (e.g., FASTA2 and FASTA3) which provides alignments for regions of optimal overlap between the challenge and query sequences and percent sequence identities (Pearson, Methods Enzymol. 183:63-98 (1990); Pearson, Methods Mol. Biol., 132:185-219 (2000)). Another preferred algorithm when comparing sequences to a database containing massive sequences from different organisms is the computer program BLAST, in particular, blastp or tblastn (using default parameters provided by the program). See, for example, Altschul et al., Mol. Biol., 215:403-410 (1990); Al tschul et al., Nucleic Acids Res., 25:3389-402 (1997).

Compared with the prior art, the present invention has the following advantages:

The anti-human IL-4RA antibody disclosed herein can bind to human IL-4RA with high affinity, and inhibit relevant cellular biological effects mediated by human IL-4 and human IL-13, such as cell proliferation, IL-4 and IL-13 induced CD23 expression level up-regulation and the like, by blocking the binding of human IL-4 and human IL-13 to human IL-4RA. The antibody has advantages of high activity, exclusion of species difference and the like, and can be used in preparing medicaments for blocking the binding of human IL-4 and IL-13 to human IL-4RA and in preparing medicaments for treating or preventing allergic diseases such as allergic rhinitis, asthma, allergy and atopic dermatitis (including moderate and severe atopic dermatitis), sinusitis, nasal polyps, chronic obstructive pulmonary disease, tissue fibrosis, and autoimmune diseases, thus having prospect for application and marketing.

### BRIEF DESCRIPTION OF THE DRAWINGS

FIG. 1: Binding activity of 13E5 and dupilumab to antigen IL4RA-mFc.

FIG. 2: Binding activity of 13E5 H1L1, 13E5 H2L2, 13E5 H3L3 and dupilumab to antigen IL4RA-mFc.

FIG. 3: Binding activity of 13E5 H4L2, 13E5 H4L4 and dupilumab to antigen IL4RA-mFc.

FIG. 4: Activity of 13E5 and dupilumab in competing with human IL4-N-His for binding to human IL4RA-mFc.

FIG. 5: Activity of 13E5 H1L1, 13E5 H2L2, 13E5 H3L3 and dupilumab in competing with human IL4-N-His for binding to human IL4RA-mFc.

FIG. 6: Activity of 13E5 H4L2, 13E5 H4L4 and dupilumab in competing with human IL4-N-His for binding to human IL4RA-mFc.

FIG. 7: Binding activity of 18H7 and dupilumab to antigen IL4RA-mFc.

FIG. 8: Binding activity of 18H7 H1L1, 18H7 H2L2, 18H7 H2L3, 18H7 H3L2, 18H7 H3L3 and dupilumab to antigen IL4RA-mFc.

FIG. 9: Activity assay of 18H7 and dupilumab in competing with human IL4-N-His for binding to human IL4RA-mFc.

FIG. 10: Activity assay of 18H7 H1L1, 18H7 H2L2, 18H7 H2L3, 18H7 H3L2 and dupilumab in competing with human IL4-N-His for binding to human IL4RA-mFc.

FIG. 11: Activity assay of 20G10 H3L3, 13E5 H4L4 and dupilumab in competing with human IL4-N-His for binding to human IL4RA-mFc.

FIG. 12: Affinity constant assay of 13E5 H4L4 to human IL4RA. The antibody concentrations for the curve pairs from top to bottom were 25 nM, 6.25 nM, 3.13 nM, 1.56 nM, 0.78 nM and 0.39 nM, respectively.

FIG. 13: Affinity constant assay of 18H7 H1L1 to human IL4RA. The antibody concentrations for the curve pairs from top to bottom were 25 nM, 6.25 nM, 3.13 nM, 1.56 nM, 0.78 nM and 0.39 nM, respectively.

FIG. 14: Affinity constant assay of dupilumab to human IL4RA. The antibody concentrations for the curve pairs from top to bottom were 12.5 nM, 6.25 nM, 3.13 nM, 1.56 nM, 0.78 nM and 0.39 nM, respectively.

FIG. 15: Affinity constant assay of 13E5 H1L1 to human IL4RA. The antibody concentrations for the curve pairs from top to bottom were 25 nM, 12.5 nM, 6.25 nM, 3.13 nM, 1.56 nM, 0.78 nM and 0.39 nM, respectively.

FIG. 16: Affinity constant assay of 13E5 H2L2 to human IL4RA. The antibody concentrations for the curve pairs from top to bottom were 25 nM, 12.5 nM, 6.25 nM, 3.13 nM, 1.56 nM, 0.78 nM and 0.39 nM, respectively.

FIG. 17: Affinity constant assay of 13E5 H3L3 to human IL4RA. The antibody concentrations for the curve pairs from top to bottom were 25 nM, 12.5 nM, 6.25 nM, 3.13 nM, 1.56 nM and 0.39 nM, respectively.

FIG. 18: Affinity constant assay of 13E5 H4L2 to human IL4RA. The antibody concentrations for the curve pairs from top to bottom were 12.5 nM, 6.25 nM, 3.13 nM, 1.56 nM, 0.78 nM and 0.39 nM, respectively.

FIG. 19: Affinity constant assay of 18H7 H2L2 to human IL4RA. The antibody concentrations for the curve pairs from top to bottom were 6.25 nM, 3.13 nM, 1.56 nM, 0.78 nM and 0.39 nM, respectively.

FIG. 20: Affinity constant assay of 18H7 H3L2 to human IL4RA. The antibody concentrations for the curve pairs from top to bottom were 25 nM, 12.5 nM, 6.25 nM, 3.13 nM, 1.56 nM, 0.78 nM and 0.39 nM, respectively.

FIG. 21: Affinity constant assay of dupilumab to human IL4RA. The antibody concentrations for the curve pairs from top to bottom were 6.25 nM, 3.13 nM, 1.56 nM, 0.78 nM and 0.39 nM, respectively.

FIG. 22: 13E5 H1L1, 13E5 H2L2 and dupilumab inhibiting IL-4-induced TF-1 cell proliferation.

FIG. 23: 13E5 H1L1, 13E5 H2L2 and dupilumab inhibiting IL-13-induced TF-1 cell proliferation.

FIG. 24: 13E5 H4L2, 13E5 H4L4 and dupilumab inhibiting IL-4-induced TF-1 cell proliferation.

FIG. 25: 13E5 H4L2, 13E5 H4L4 and dupilumab inhibiting IL-13-induced TF-1 cell proliferation.

FIG. 26: 18H7 H1L1, 18H7 H2L2 and dupilumab inhibiting IL-4-induced TF-1 cell proliferation.

FIG. 27: 18H7 H1L1, 18H7 H2L2 and dupilumab inhibiting IL-13-induced TF-1 cell proliferation.

FIG. 28: 18H7 H1L1, 13E5 H4L4 and dupilumab inhibiting IL-4-induced CD23 expression up-regulation in monocytes.

FIG. 29: 18H7 H1L1, 13E5 H4L4 and dupilumab inhibiting IL-13-induced CD23 expression up-regulation in monocytes.

FIG. 30: 13E5 H4L4 inhibiting increase of epidermal thickness in B-hIL4Ra mouse skin inflammation model.

FIG. 31: Pathological sections showing epidermal thickness in mice of all experimental groups (HE staining, 400×). a. control (normal saline); b. dupilumab (80 mg/kg); c. dupilumab (20 mg/kg); d. hIgG4 (80 mg/kg); e. 13E5 H4L4 (80 mg/kg); f. 13E5 H4L4 (20 mg/kg).

### DETAILED DESCRIPTION

The embodiments of the present invention will be described in detail below with reference to the examples. Those skilled in the art will understand that the following examples are only used to illustrate the present invention, and should not be regarded as limiting the scope of the present invention. In the cases where the techniques or conditions are not specified, the examples were carried out according to the techniques or conditions described in the literature in the art (e.g., see, Molecular Cloning: A Laboratory Manual, authored by J. Sambrook et al., and translated by Huang Peitang et al., Third Edition, Science Press) or according to the product manual. Reagents or instruments used are commercially available conventional products if the manufacturers thereof are not specified.

In the following examples of the present invention, BALB/C mice were purchased from Guangdong Medical Laboratory Animal Center.

In the following examples of the present invention, the reference antibody dupilumab VAB 16F3-1 (hereinafter referred to as dupilumab) was produced by Akeso, Inc., the sequence of which can be found in Patent Application No. PCT/US2007/021210 granted to Regeneron Pharmaceuticals, Inc. The heavy chain variable region of antibody VAB 16F3-1 is set forth in SEQ ID NO: 124, with the constant region being an Ig gamma-1 chain C region, ACCESSION No. P01857, while the coding sequence of the light chain variable region of antibody VAB 16F3-1 is set forth in SEQ ID NO: 125, with the constant region being an Ig kappa chain C region, ACCESSION No. P01834).

### Example 1. Preparation of Anti-Human IL-4RA Antibodies 20G10, 13E5 and 18H7

### 1. Preparation of Hybridoma Cell Lines 20G10, 13E5 and 18H7

The antigen IL-4RA-mFc for producing anti-IL-4RA antibody was a fusion protein of human IL-4RA mature peptide (Genbank ID: NP_001244336.1) and mFc tag (SEQ ID NO: 121) synthesized by Akeso, Inc., and was used for immunizing BALB/C mice (purchased from Guangdong Medical Laboratory Animal Center). Spleen cells of immunized BALB/C mice (purchased from Guangdong center for medicine laboratory animals) and mouse myeloma cells were fused to form hybridoma cells with reference to existing cell fusion techniques (e.g., Stewart, S.J., “Monoclonal Antibody Production”, in Basic Methods in Antibody Production and Characterization, Eds. G.C. Howard and D.R. Bethell, Boca Raton: CRC Press, 2000). The plate was coated with IL 4RA-hFc protein (IL-4RA is described above, and hFc is a human IgG Fc purification tag, specifically Ig gamma-1 chain C region, Genbank ID: P01857, positions 114-330) for indirect ELISA. By screening, hybridoma cell lines secreting antibodies specifically binding to IL 4RA-hFc were obtained. The hybridoma cell lines obtained by indirect ELISA screening were subjected to competitive ELISA to select hybridoma cell lines secreting monoclonal antibodies that compete with ligand IL4-N-his (IL4 NCBI Gene ID: AAH70123.1) for binding to IL 4RA-hFc. Two hybridoma cell lines stably secreting anti-human IL-4RA antibodies were obtained by limiting dilution. The hybridoma cell lines were named as LT018, LT008 and LT009, and the secreted monoclonal antibodies were named as 20G10, 13E5 and 18H7.

Hybridoma cell line LT018 (IL4RA-20G10) was deposited at China Center for Type Culture Collection (CCTCC) on Dec. 25, 2019 with an accession number CCTCC NO: C202010, and a preservation address of Wuhan University, Wuhan, China, postal code: 430072.

Hybridoma cell line LT008 (IL4RA-13E5) was deposited at China Center for Type Culture Collection (CCTCC) on Jun. 21, 2018 with an accession number CCTCC NO: C2018131, and a preservation address of Wuhan University, Wuhan, China, postal code: 430072.

Hybridoma cell line LT009 (IL4RA-18H7) was deposited at China Center for Type Culture Collection (CCTCC) on Jun. 21, 2018 with an accession number CCTCC NO: C2018132, and a preservation address of Wuhan University, Wuhan, China, postal code: 430072.

### 2. Preparation of Anti-Human IL-4RA Antibodies 20G10, 13E5 and 18H7

The cell lines LT018, LT008 and LT009 prepared as described above were separately cultured with a chemical defined medium (CD medium; containing 1% streptomycin) in a 5% CO₂, 37° C. incubator. After 7 days, the supernatants were collected and purified by high-speed centrifugation, vacuum filtration through a microfiltration membrane and a HiTrap protein A HP column to give antibodies 20G10, 13E5 and 18H7.

### Example 2. Sequence Analysis of Anti-Human IL-4RA Antibody 13E5

mRNA was extracted from the cell line LT008 cultured in Example 1 according to the method described in the manual of RNAprep pure Cell/Bacteria Kit (Tiangen, Cat. No. DP430).

cDNA was synthesized according to the manual of Invitrogen SuperScript® III First-Strand Synthesis System for RT-PCR and amplified by PCR.

The PCR-amplified products were directly subjected to TA cloning according to the manual of the pEASY-T1 Cloning Kit (Transgen CT101).

The TA-cloned products were directly sequenced, and the sequencing results are as follows:

The nucleic acid sequence of the heavy chain variable region is set forth in SEQ ID NO: 1 with a length of 348 bp. The coded amino acid sequence is set forth in SEQ ID NO: 2 with a length of 116 amino acids, while the sequences of heavy chain CDR1, CDR2 and CDR3 are set forth in SEQ ID NOs: 9, 10 and 11, respectively.

The nucleic acid sequence of the light chain variable region is set forth in SEQ ID NO: 3 with a length of 321 bp. The coded amino acid sequence is set forth in SEQ ID NO: 4 with a length of 107 amino acids, while the sequences of light chain CDR1, CDR2 and CDR3 are set forth in SEQ ID NOs: 12, 13 and 14, respectively.

### Example 3. Design and Preparation of Humanized Anti-Human IL-4RA Antibodies 13E5 H1L1, 13E5 H2L2, 13E5 H3L3, 13E5 H4L2 and 13E5 H4L4

### 1. Design of Light and Heavy Chain Sequences of Humanized Anti-IL4RA Antibodies 13E5 H1L1, 13E5 H2L2, 13E5 H3L3, 13E5 H4L2, and 13E5 H4L4

Based on the three-dimensional crystal structure of human IL-4RA protein (Hage T, Reinemer P, Sebald W., Crystals of a 1:1 Complex Between Human Interleukin-4 and the Extracellular Domain of Its Receptor Alpha Chain, Eur. J. Biochem., 1998; 258(2):831-6.) and the sequence of antibody 13E5 obtained in Example 2, the variable region sequences of antibodies 13E5 H1L1, 13E5 H2L2, 13E5 H3L3, 13E5 H4L2 and 13E5 H4L4 were given by computer modeling and mutation design (antibody constant region sequences from NCBI database: the heavy chain constant region is Ig gamma-4 chain C region, ACCESSION No. P01861.1; the light chain constant region is Ig kappa chain C region, ACCESSION No. P01834).

The designed variable region sequences are as follows:

### Heavy and Light Chain Sequences of Humanized Monoclonal Antibody 13E5 H1L1

The nucleic acid sequence of the heavy chain variable region is set forth in SEQ ID NO: 21 with a length of 348 bp. The coded amino acid sequence is set forth in SEQ ID NO: 22 with a length of 116 amino acids, while the sequences of heavy chain CDR1, CDR2 and CDR3 are set forth in SEQ ID NOs: 9, 10 and 11, respectively.

The nucleic acid sequence of the light chain variable region is set forth in SEQ ID NO: 23 with a length of 321 bp. The coded amino acid sequence is set forth in SEQ ID NO: 24 with a length of 107 amino acids, while the sequences of light chain CDR1, CDR2 and CDR3 are set forth in SEQ ID NOs: 12, 13 and 14, respectively.

### Heavy and Light Chain Sequences of Humanized Monoclonal Antibody 13E5 H2L2

The nucleic acid sequence of the heavy chain variable region is set forth in SEQ ID NO: 25 with a length of 348 bp. The coded amino acid sequence is set forth in SEQ ID NO: 26 with a length of 116 amino acids, while the sequences of heavy chain CDR1, CDR2 and CDR3 are set forth in SEQ ID NOs: 9, 10 and 11, respectively.

The nucleic acid sequence of the light chain variable region is set forth in SEQ ID NO: 27 with a length of 321 bp. The coded amino acid sequence is set forth in SEQ ID NO: 28 with a length of 107 amino acids, while the sequences of light chain CDR1, CDR2 and CDR3 are set forth in SEQ ID NOs: 12, 13 and 14, respectively.

### Heavy and Light Chain Sequences of Humanized Monoclonal Antibody 13E5 H3L3

The nucleic acid sequence of the heavy chain variable region is set forth in SEQ ID NO: 29 with a length of 348 bp. The coded amino acid sequence is set forth in SEQ ID NO: 30 with a length of 116 amino acids, while the sequences of heavy chain CDR1, CDR2 and CDR3 are set forth in SEQ ID NOs: 9, 10 and 11, respectively.

The nucleic acid sequence of the light chain variable region is set forth in SEQ ID NO: 31 with a length of 321 bp. The coded amino acid sequence is set forth in SEQ ID NO: 32 with a length of 107 amino acids, while the sequences of light chain CDR1, CDR2 and CDR3 are set forth in SEQ ID NOs: 12, 13 and 14, respectively.

### Heavy and Light Chain Sequences of Humanized Monoclonal Antibody 13E5 H4L4

The nucleic acid sequence of the heavy chain variable region is set forth in SEQ ID NO: 33 with a length of 348 bp. The coded amino acid sequence is set forth in SEQ ID NO: 34 with a length of 116 amino acids, while the sequences of heavy chain CDR1, CDR2 and CDR3 are set forth in SEQ ID NOs: 9, 10 and 11, respectively.

The nucleic acid sequence of the light chain variable region is set forth in SEQ ID NO: 35 with a length of 321 bp. The coded amino acid sequence is set forth in SEQ ID NO: 36 with a length of 107 amino acids, while the sequences of light chain CDR1, CDR2 and CDR3 are set forth in SEQ ID NOs: 12, 13 and 14, respectively.

### Heavy and Light Chain Sequences of Humanized Monoclonal Antibody 13E5 H4L2

The nucleic acid sequence of the heavy chain variable region is set forth in SEQ ID NO: 33, and

the coded amino acid sequence is set forth in SEQ ID NO: 34.

The nucleic acid sequence of the light chain variable region is set forth in SEQ ID NO: 27, and

the coded amino acid sequence is set forth in SEQ ID NO: 28.

### 2. Preparation of Humanized Antibodies 13E5 H1L1, 13E5 H2L2, 13E5 H3L3, 13E5 H4L2 and 13E5 H4L4

The heavy chain constant regions are Ig gamma-4 chain C region, ACCESSION: P01861.1; the light chain constant regions are Ig kappa chain C region, ACCESSION: P01834.

The heavy and light chain variable region cDNA of 13E5 H1L1, heavy and light chain variable region cDNA of 13E5 H2L2, heavy and light chain variable region cDNA of 13E5 H3L3, heavy and light chain variable region cDNA of 13E5 H4L2, and heavy and light chain variable region cDNA of 13E5 H4L4 were separately cloned into pUC57 simple vector (provided by Genscript Biotech Corporation) to give pUC57simple-13E5H1, pUC57simple-13E5L1, pUC57simple-13E5H2, pUC57simple-13E5L2, pUC57simple-13E5H3, pUC57simple-13E5L3, pUC57simple-13E5H4 and pUC57simple-13E5L4. The variable region fragments were acquired by digestion according to the standard techniques described in Molecular Cloning: A Laboratory Manual (Second Edition), and subcloned into the pcDNA3.1 vectors containing the corresponding heavy or light chain constant region fragment (for the pcDNA3.1 vectors containing the heavy and light chain constant regions, the restriction enzymes were HindIII & EcoRI) to give pcDNA3.1-13E5H1, pcDNA3.1-13E5L1, pcDNA3.1-13E5H2, pcDNA3.1-13E5L2, pcDNA3.1-13E5H3, pcDNA3.1-13E5L3, pcDNA3.1-13E5H4 and pcDNA3.1-13E5L4. The recombinant plasmid pair containing the corresponding light and heavy chains (pcDNA3.1-13E5H1 and pcDNA3.1-13E5L1, pcDNA3.1-13E5H2 and pcDNA3.1-13E5L2, pcDNA3.1-13E5H3 and pcDNA3.1-13E5L3, pcDNA3.1-13E5H4 and pcDNA3.1-13E5L4, and pcDNA3.1-13E5H4 and pcDNA3.1-13E5L2) were co-transfected into 293F cells. The cultures were collected and purified. After the sequences were verified, endotoxin-free expression plasmids were prepared, and were transiently transfected into HEK293 cells for antibody expression. The cultures were collected after 7 days, and purified on a Protein A column to give humanized antibodies 13E5 H1L1, 13E5 H2L2, 13E5 H3L3, 13E5 H4L2 and 13E5 H4L4.

### Example 4. Sequence Analysis of Anti-Human IL-4RA Antibody 18H7

mRNA was extracted from the cell line LT009 cultured in Example 1 according to the method described in the manual of RNAprep pure Cell/Bacteria Kit (Tiangen, Cat. No. DP430).

cDNA was synthesized according to the manual of Invitrogen SuperScript® III First-Strand Synthesis System for RT-PCR and amplified by PCR.

The PCR-amplified products were directly subjected to TA cloning according to the manual of the pEASY-T1 Cloning Kit (Transgen CT101).

The TA-cloned products were directly sequenced, and the sequencing results are as follows:

The nucleic acid sequence of the heavy chain variable region is set forth in SEQ ID NO: 5 with a length of 360 bp. The coded amino acid sequence is set forth in SEQ ID NO: 6 with a length of 120 amino acids, while the sequences of heavy chain CDR1, CDR2 and CDR3 are set forth in SEQ ID NOs: 15, 16 and 17, respectively.

The nucleic acid sequence of the light chain variable region is set forth in SEQ ID NO: 7 with a length of 333 bp. The coded amino acid sequence is set forth in SEQ ID NO: 8 with a length of 111 amino acids, while the sequences of light chain CDR1, CDR2 and CDR3 are set forth in SEQ ID NOs: 18, 19 and 20, respectively.

### Example 5. Design and Preparation of Humanized Anti-Human IL-4RA Antibodies 18H7 H1L1, 18H7 H2L2, 18H7 H2L3, 18H7 H3L2 and 18H7 H3L3

### 1. Design of Light and Heavy Chain Sequences of Humanized Anti-IL-4RA Antibodies 18H7 H1L1, 18H7 H2L2, 18H7 H2L3, 18H7 H3L2, and 18H7 H3L3

Based on the three-dimensional crystal structure of human IL-4RA protein (Nat Hage T, Reinemer P, Sebald W., Crystals of a 1:1 Complex Between human Interleukin-4 and the Extracellular Domain of Its Receptor Alpha Chain, Eur. J. Biochem., 1998; 258(2):831-6.) and the sequence of antibody 18H7 obtained in Example 4, the variable region sequences of antibodies 18H7 H1L1, 18H7 H2L2, 18H7 H2L3, 18H7 H3L2 and 18H7 H3L3 were given by computer modeling and mutation design (antibody constant region sequences from NCBI database: the heavy chain constant region is Ig gamma-4 chain C region, ACCESSION No. P01861.1; the light chain constant region is Ig kappa chain C region, ACCESSION No. P01834).

The designed variable region sequences are as follows:

### Heavy and Light Chain Sequences of Humanized Monoclonal Antibody 18H7 H1L1

The nucleic acid sequence of the heavy chain variable region is set forth in SEQ ID NO: 37 with a length of 360 bp. The coded amino acid sequence is set forth in SEQ ID NO: 38 with a length of 120 amino acids, while the sequences of heavy chain CDR1, CDR2 and CDR3 are set forth in SEQ ID NOs: 15, 16 and 17, respectively.

The nucleic acid sequence of the light chain variable region is set forth in SEQ ID NO: 39 with a length of 333 bp. The coded amino acid sequence is set forth in SEQ ID NO: 40 with a length of 111 amino acids, while the amino acid sequences of light chain CDR1, CDR2 and CDR3 are set forth in SEQ ID NOs: 18, 19 and 20, respectively.

### Heavy and Light Chain Sequences of Humanized Monoclonal Antibody 18H7 H2L2

The nucleic acid sequence of the heavy chain variable region is set forth in SEQ ID NO: 41 with a length of 360 bp. The coded amino acid sequence is set forth in SEQ ID NO: 42 with a length of 120 amino acids, while the sequences of heavy chain CDR1, CDR2 and CDR3 are set forth in SEQ ID NOs: 15, 16 and 17, respectively.

The nucleic acid sequence of the light chain variable region is set forth in SEQ ID NO: 43 with a length of 333 bp. The coded amino acid sequence is set forth in SEQ ID NO: 44 with a length of 111 amino acids, while the sequences of light chain CDR1, CDR2 and CDR3 are set forth in SEQ ID NOs: 18, 19 and 20, respectively.

### Heavy and Light Chain Sequences of Humanized Monoclonal Antibody 18H7 H3L3

The nucleic acid sequence of the heavy chain variable region is set forth in SEQ ID NO: 45 with a length of 360 bp. The coded amino acid sequence is set forth in SEQ ID NO: 46 with a length of 120 amino acids, while the sequences of heavy chain CDR1, CDR2 and CDR3 are set forth in SEQ ID NOs: 15, 16 and 17, respectively.

The nucleic acid sequence of the light chain variable region is set forth in SEQ ID NO: 47 with a length of 333 bp. The coded amino acid sequence is set forth in SEQ ID NO: 48 with a length of 111 amino acids, while the sequences of light chain CDR1, CDR2 and CDR3 are set forth in SEQ ID NOs: 18, 19 and 20, respectively.

### Heavy and Light Chain Sequences of Humanized Monoclonal Antibody 18H7 H2L3

The nucleic acid sequence of the heavy chain variable region is set forth in SEQ ID NO: 41, and

the coded amino acid sequence is set forth in SEQ ID NO: 42.

The nucleic acid sequence of the light chain variable region is set forth in SEQ ID NO: 47, and

the coded amino acid sequence is set forth in SEQ ID NO: 48.

### Heavy and Light Chain Sequences of Humanized Monoclonal Antibody 18H7 H3L2

The nucleic acid sequence of the heavy chain variable region is set forth in SEQ ID NO: 45, and

the coded amino acid sequence is set forth in SEQ ID NO: 46.

The nucleic acid sequence of the light chain variable region is set forth in SEQ ID NO: 43, and

the coded amino acid sequence is set forth in SEQ ID NO: 44.

### 2. Preparation of Humanized Antibodies 18H7 H1L1, 18H7 H2L2, 18H7 H2L3, 18H7 H3L3 and 18H7 H3L2

The heavy chain constant regions are Ig gamma-4 chain C region, ACCESSION: P01861.1; the light chain constant regions are Ig kappa chain C region, ACCESSION: P01834.

The heavy and light chain variable region cDNA of 18H7 H1L1, heavy and light chain variable region cDNA of 18H7 H2L2, heavy and light chain variable region cDNA of 18H7 H2L3, heavy and light chain variable region cDNA of 18H7 H3L3, and heavy and light chain variable region cDNA of 18H7 H3L2 were separately cloned into pUC57 simple vector (provided by Genscript Biotech Corporation) to give pUC57simple-18H7H1, pUC57simple-18H7L1, pUC57simple-18H7H2, pUC57simple-18H7L2, pUC57simple-18H7H3 and pUC57simple-18H7L3. The variable region fragments were acquired by digestion according to the standard techniques described in Molecular Cloning: A Laboratory Manual (Second Edition), and subcloned into the pcDNA3.1 vectors containing the corresponding heavy or light chain constant region fragment to give pcDNA3.1-18H7H1, pcDNA3.1-18H7L1, pcDNA3.1-18H7H2, pcDNA3.1-18H7L2, pcDNA3.1-18H7H3 and pcDNA3.1-18H7L3. The recombinant plasmid pair containing the corresponding light and heavy chains (pcDNA3.1-18H7H1 and pcDNA3.1-18H7L1, pcDNA3.1-18H7H2 and pcDNA3.1-18H7L2, pcDNA3.1-18H7H3 and pcDNA3.1-18H7L3, pcDNA3.1-18H7H2 and pcDNA3.1-18H7L3, and pcDNA3.1-18H7H3 and pcDNA3.1-18H7L2) were co-transfected into 293F cells. The cultures were collected and purified. After the sequences were verified, endotoxin-free expression plasmids were prepared, and were transiently transfected into HEK293 cells for antibody expression. The cultures were collected after 7 days, and purified on a Protein A column (GE) to give the humanized antibodies 18H7 H1L1, 18H7 H2L2, 18H7 H2L3, 18H7 H3L2 and 18H7 H3L3.

### Example 6. Design and Preparation of Humanized Anti-Human IL-4RA Antibody 20G10 H3L3

### 1. Design of Light and Heavy Chain Sequences of Humanized Anti-IL-4RA Antibody 20G10 H3L3

Based on the three-dimensional crystal structure of human IL-4RA protein (Nat Hage T, Reinemer P, Sebald W., Crystals of a 1:1 Complex Between human Interleukin-4 and the Extracellular Domain of Its Receptor Alpha Chain, Eur. J. Biochem., 1998; 258(2):831-6.), the variable region sequences of antibody 20G10 H3L3 were given by computer modeling and mutation design (antibody constant region sequences from NCBI database: the heavy chain constant region is Ig gamma-4 chain C region, ACCESSION No. P01861.1; the light chain constant region is Ig kappa chain C region, ACCESSION No. P01834).

The designed variable region sequences are as follows:

### Heavy and Light Chain Sequences of Humanized Monoclonal Antibody 20G10 H3L3

The nucleic acid sequence of the heavy chain variable region is set forth in SEQ ID NO: 126 with a length of 360 bp. The coded amino acid sequence is set forth in SEQ ID NO: 127 with a length of 120 amino acids, while the sequences of heavy chain CDR1, CDR2 and CDR3 are set forth in SEQ ID NO: 130, 131 and 132, respectively.

The nucleic acid sequence of the light chain variable region is set forth in SEQ ID NO: 128 with a length of 321 bp. The coded amino acid sequence is set forth in SEQ ID NO: 129 with a length of 107 amino acids, while the amino acid sequences of light chain CDR1, CDR2 and CDR3 are set forth in SEQ ID NOs: 133, 134 and 135, respectively.

### 2. Preparation of Humanized Antibody 20G10 H3L3

The heavy chain constant regions are Ig gamma-4 chain C region, ACCESSION: P01861.1; the light chain constant regions are Ig kappa chain C region, ACCESSION: P01834.

The heavy and light chain variable region cDNA of 20G10 H3L3 were cloned into pUC57simple vector (provided by Genscript Biotech Corporation) to give pUC57simple-20G10H3 and pUC57simple-20G10L3. The variable region fragments were acquired by digestion according to the standard techniques described in Molecular Cloning: A Laboratory Manual (Second Edition), and subcloned into the pcDNA3.1 vectors containing the corresponding heavy or light chain constant region fragment to give pcDNA3.1-20G10H3 and pcDNA3.1-20G10L3. The recombinant plasmid pair containing the corresponding light and heavy chains (pcDNA3.1-20G10H3 and pcDNA3.1-20G10L3) were co-transfected into 293F cells. The cultures were collected and purified. After the sequences were verified, endotoxin-free expression plasmids were prepared, and were transiently transfected into HEK293 cells for antibody expression. The cultures were collected after 7 days, and purified on a Protein A column (GE) to give the humanized antibody 20G10 H3L3.

### Example 7. Binding Activity of Antibodies to Antigens by ELISA

### 1. Binding Activity of Murine Antibody 13E5 and Humanized Antibodies 13E5 H1L1, 13E5 H2L2, 13E5 H3L3, 13E5 H4L2 and 13E5 H4L4 to Antigens Human IL4RA-hFc or Human IL-4RA-mFc

1.1 The binding activity of antibodies 13E5, 13E5 H1L1, 13E5 H2L2, 13E5 H3L3, 13E5 H4L2 and 13E5 H4L4 to the antigens human IL4RA-hFc or human IL4RA-mFc was measured by indirect ELISA.

Procedures: The ELISA plates were coated with human IL4RA-hFc or human IL4RA-mFc and were incubated, and target antibodies were added after blocking. Goat anti-mouse IgG (H+L) and HRP (purchased from Jackson ImmunoResearch Inc., Cat No. 109-035-062), or goat anti-human IgG and HRP (purchased from Jackson ImmunoResearch Inc., Cat No. 109-035-088) were added. The plates were incubated and washed before TMB (Neogen, 308177) was added for chromogenic reaction. At the end of the reaction, the absorbance at 450 nm was measured by a plate reader. The data were analyzed by SoftMax Pro 6.2.1.

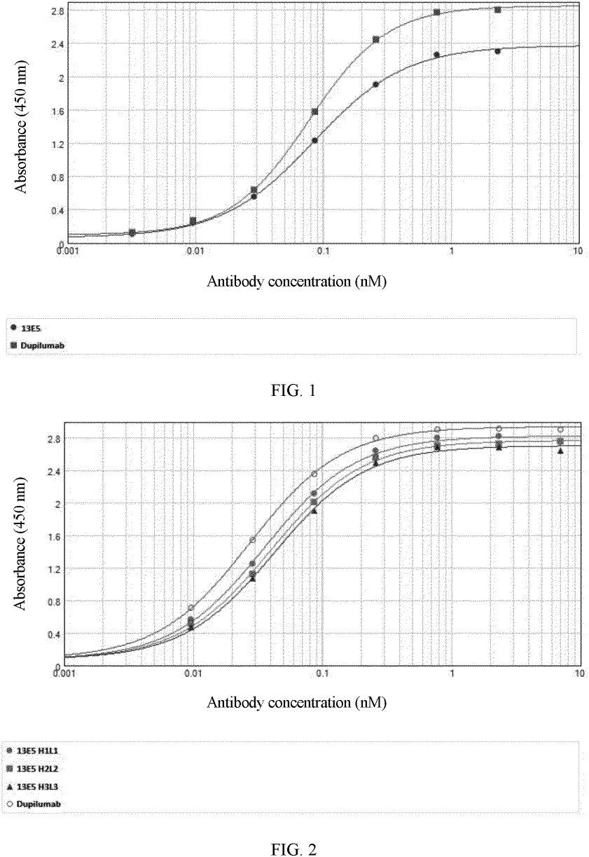

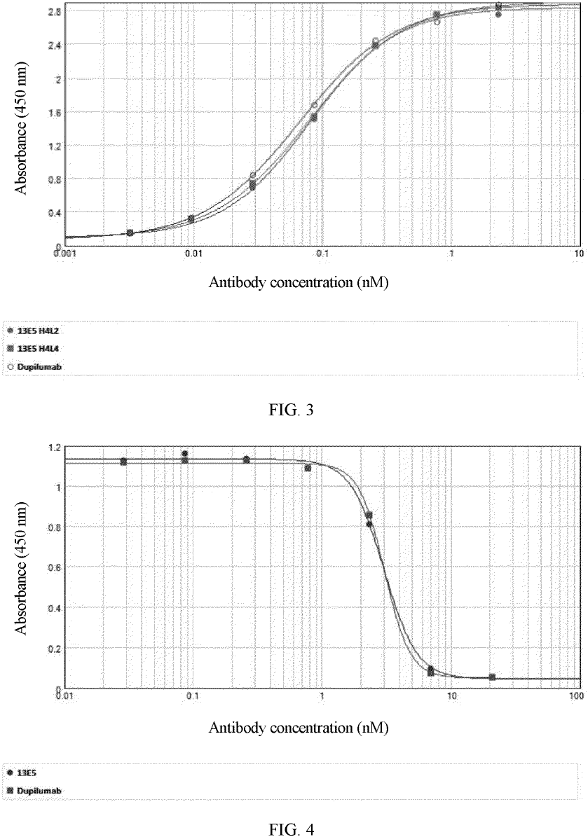

The readings at 450 nm show that antibodies 13E5, 13E5 H1L1, 13E5 H2L2, 13E5 H3L3, 13E5 H4L2 and 13E5 H4L4 are all capable of effectively binding to antigens human IL4RA-hFc or human IL4RA-mFc in a dose-dependent manner. By 4-parameter logistic regression of absorbance vs. antibody concentration, as shown in Tables 1, 2 and 3, 13E5, 13E5 H1L1, 13E5 H2L2, 13E5 H3L3, 13E5 H4L2 and 13E5 H4L4 are all capable of binding to antigens human IL4RA-hFc or human IL4RA-mFc effectively, and demonstrate comparable binding activities to that of the reference antibody dupilumab against the same target (FIGS. 1, 2 and 3).

TABLE 1

Binding activity of 13E5 and dupilumab to antigens human IL4RA-hFc or human IL4RA-mFc

| Antibody dilution (µg/mL) | Antigen (1 µg/mL) | | | |
|---|---|---|---|---|
| | IL4RA-hFc | | IL4RA-mFc | |
| | 13E5 | | Dupilumab | |
| 0.333 | 2.300 | 2.313 | 2.758 | 2.858 |
| 1:3 | 2.318 | 2.215 | 2.748 | 2.801 |
| 1:9 | 1.923 | 1.878 | 2.388 | 2.500 |
| 1:27 | 1.251 | 1.207 | 1.469 | 1.679 |
| 1:81 | 0.558 | 0.563 | 0.599 | 0.673 |
| 1:243 | 0.244 | 0.237 | 0.250 | 0.281 |
| 1:729 | 0.112 | 0.113 | 0.126 | 0.137 |
| 0 | 0.047 | 0.053 | 0.076 | 0.069 |

| | | |
|---|---|---|
| Second antibody | Goat anti-mouse IgG (H+L), HRP (1:5000) | Goat anti-human IgG (H+L), HRP (1:5000) |
| EC₅₀ (nM) | 0.084 | 0.077 |

TABLE 2

Binding activity of 13E5 H1L1, 13E5 H2L2, 13E5 H3L3 and dupilumab to antigen IL4RA-mFc

| Antibody dilution (µg/mL) | Antigen: IL4RA-mFc | | | | | | | |
|---|---|---|---|---|---|---|---|---|
| | 13E5 H1L1 | | 13E5 H2L2 | | 13E5 H3L3 | | Dupilumab | |
| 1 | 2.716 | 2.797 | 2.768 | 2.754 | 2.642 | 2.656 | 2.901 | 2.921 |
| 1:3 | 2.834 | 2.825 | 2.686 | 2.770 | 2.679 | 2.688 | 2.928 | 2.923 |
| 1:9 | 2.802 | 2.798 | 2.692 | 2.735 | 2.658 | 2.717 | 2.909 | 2.921 |
| 1:27 | 2.684 | 2.613 | 2.551 | 2.578 | 2.492 | 2.498 | 2.799 | 2.801 |
| 1:81 | 2.116 | 2.122 | 1.972 | 2.043 | 1.918 | 1.891 | 2.358 | 2.358 |
| 1:243 | 1.214 | 1.297 | 1.104 | 1.153 | 1.069 | 1.080 | 1.551 | 1.544 |
| 1:729 | 0.571 | 0.566 | 0.499 | 0.528 | 0.469 | 0.464 | 0.717 | 0.705 |
| 0 | 0.056 | 0.059 | 0.055 | 0.056 | 0.056 | 0.056 | 0.056 | 0.093 |

| | | | | |
|---|---|---|---|---|
| Second antibody | Goat anti-human IgG (H+L), HRP (1:5000) | | | |
| EC₅₀ (nM) | 0.035 | 0.040 | 0.042 | 0.028 |

TABLE 3

Binding activity of 13E5 H4L2, 13E5 H4L4 and dupilumab to antigen IL4RA-mFc

| Antibody dilution (µg/mL) | Antigen: IL4RA-mFc | | | | | |
|---|---|---|---|---|---|---|
| | 13E5 H4L2 | | 13E5 H4L4 | | Dupilumab | |
| 0.333 | 2.733 | 2.772 | 2.798 | 2.879 | 2.850 | 2.893 |
| 1:3 | 2.730 | 2.775 | 2.766 | 2.730 | 2.676 | 2.673 |
| 1:9 | 2.359 | 2.406 | 2.370 | 2.392 | 2.482 | 2.408 |
| 1:27 | 1.492 | 1.523 | 1.474 | 1.608 | 1.637 | 1.722 |
| 1:81 | 0.673 | 0.707 | 0.709 | 0.778 | 0.861 | 0.816 |
| 1:243 | 0.305 | 0.293 | 0.317 | 0.313 | 0.335 | 0.336 |
| 1:729 | 0.145 | 0.134 | 0.146 | 0.147 | 0.147 | 0.147 |
| 0 | 0.063 | 0.063 | 0.064 | 0.064 | 0.067 | 0.064 |

| | | | |
|---|---|---|---|
| Second antibody | Goat anti-human IgG (H+L), HRP (1:5000) | | |
| EC₅₀ (nM) | 0.079 | 0.079 | 0.066 |

1.2 The binding activity of antibodies 13E5, 13E5 H1L1, 13E5 H2L2, 13E5 H3L3, 13E5 H4L2 and 13E5 H4L4 for blocking the binding between IL4-N-his and the antigen IL4RA-hFc was measured by competitive ELISA.

The EC₅₀ (median effect concentration) of 13E5, 13E5 H1L1, 13E5 H2L2, 13E5 H3L3, 13E5 H4L2 and 13E5 H4L4 for competing with ligand human IL4-N-His for binding to the antigen human IL4RA-hFc was assayed by ELISA, so as to investigate the activity of 13E5, 13E5 H1L1, 13E5 H2L2, 13E5 H3L3, 13E5 H4L2 and 13E5 H4L4 for blocking the binding of the ligand to the target antigen human IL4RA-hFc.

The ELISA plates were coated with human IL4RA-hFc and blocked before the antibodies were added. An equal volume of human IL4-N-His (synthesized by Akeso, Inc.) was added, and the mixture was well mixed and incubated. After the plates were washed, mouse anti-His, HRP (purchased from CoWin Biosciences, Cat No. CW0285A) was added for incubation. The plates were subjected to another wash. Chromogenic reaction was started by adding TMB (Neogen, 308177) and was then terminated. At the end of the reaction, the OD value at 450 nm wavelength was measured by a plate reader, and was analyzed and processed by SoftMax Pro 6.2.1. The results are shown in Tables 4, 5 and 6.

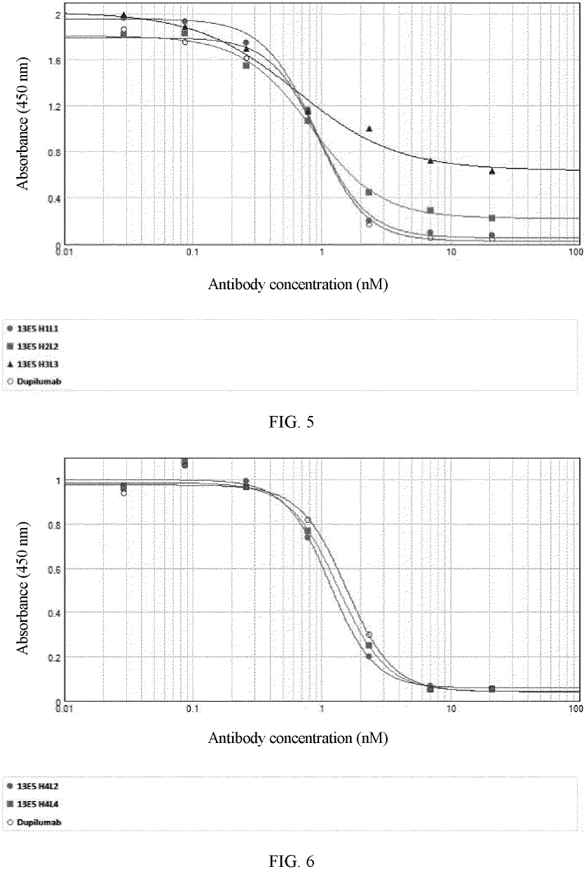

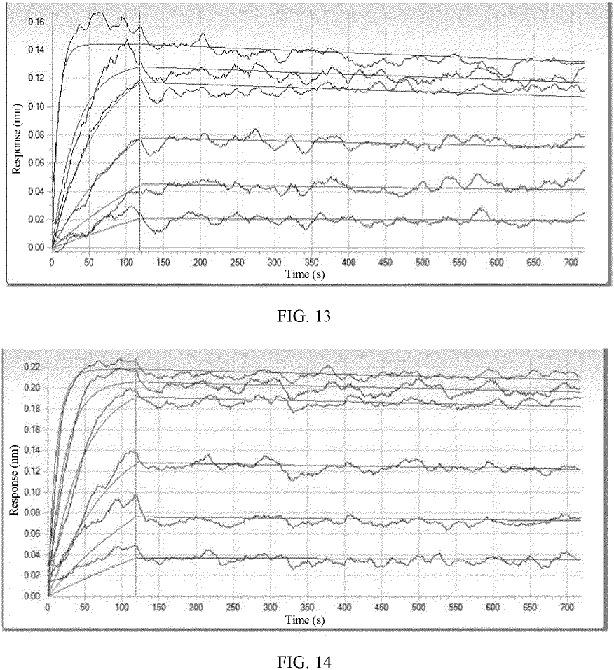

By 4-parameter logistic regression of absorbance vs. antibody concentration, the blocking EC₅₀ of the antibodies was calculated. As shown in FIGS. 4, 5 and 6, 13E5, 13E5 H1L1, 13E5 H2L2, 13E5 H4L2 and 13E5 H4L4 are all capable of effectively blocking the binding of the ligand human IL4-N-His to the antigen human IL4RA-hFc in a dose-dependent manner. Antibodies 13E5 H1L1, 13E5 H2L2, 13E5 H4L2 and 13E5 H4L4 demonstrate superior activities for competing with human IL4-N-His for binding to human IL4RA-hFc as compared to that of the reference antibody dupilumab for the same antigen.

TABLE 4

Activity assay of 13E5 and dupilumab for competing with human IL4-N-His for binding to human IL4RA-hFc

| Antibody dilution (µg/mL) | Antigen: IL4RA-hFc | | | |
|---|---|---|---|---|
| | 13E5 | | Dupilumab | |
| 3 | 0.053 | 0.053 | 0.054 | 0.052 |
| 1:3 | 0.093 | 0.100 | 0.074 | 0.076 |
| 1:9 | 0.790 | 0.832 | 0.837 | 0.877 |
| 1:27 | 1.080 | 1.095 | 1.056 | 1.123 |
| 1:81 | 1.108 | 1.164 | 1.100 | 1.149 |
| 1:243 | 1.120 | 1.199 | 1.126 | 1.126 |
| 1:729 | 1.125 | 1.129 | 1.076 | 1.158 |
| 0 | 1.136 | 1.156 | 1.123 | 1.093 |

IL4-N-his

| | | |
|---|---|---|
| Second antibody | Mouse anti-His, HRP (1:4000) | |
| EC₅₀ (nM) | 3.000 | 3.033 |

TABLE 5

Activity assay of 13E5 H1L1, 13E5 H2L2, 13E5 H3L3 and dupilumab for competing with human IL4-N-His for binding to human IL4RA-hFc

| Antibody dilution (µg/mL) | Antigen: IL4RA-hFc | | | | | | | |
|---|---|---|---|---|---|---|---|---|
| | 13E5 H1L1 | | 13E5 H2L2 | | 13E5 H3L3 | | Dupilumab | |
| 3 | 0.070 | 0.078 | 0.230 | 0.216 | 0.645 | 0.620 | 0.050 | 0.049 |
| 1:3 | 0.095 | 0.103 | 0.282 | 0.306 | 0.727 | 0.708 | 0.059 | 0.057 |
| 1:9 | 0.179 | 0.225 | 0.443 | 0.455 | 1.047 | 0.955 | 0.186 | 0.159 |
| 1:27 | 0.921 | 1.415 | 1.048 | 1.096 | 1.450 | 0.845 | 1.230 | 1.053 |
| 1:81 | 1.468 | 2.037 | 1.562 | 1.543 | 1.705 | 1.686 | 1.680 | 1.546 |
| 1:243 | 1.705 | 2.157 | 1.776 | 1.888 | 1.932 | 1.846 | 1.774 | 1.743 |
| 1:729 | 1.746 | 2.182 | 1.792 | 1.845 | 2.060 | 1.933 | 1.889 | 1.841 |
| 0 | 1.793 | 2.243 | 1.741 | 1.776 | 2.056 | 1.912 | 1.828 | 1.806 |

IL4-N-his

| | | | | |
|---|---|---|---|---|
| Second antibody | Mouse anti-His, HRP (1:4000) | | | |
| EC₅₀(nM) | 0.875 | 0.819 | 0.636 | 0.943 |

TABLE 6

Activity assay of 13E5 H4L2, 13E5 H4L4 and dupilumab for competing with human IL4-N-His for binding to human IL4RA-hFc

| Antibody dilution (µg/mL) | Antigen: IL4RA-hFc | | | | | |
|---|---|---|---|---|---|---|
| | 13E5 H4L2 | | 13E5 H4L4 | | Dupilumab | |
| 3 | 0.056 | 0.059 | 0.054 | 0.051 | 0.054 | 0.057 |
| 1:3 | 0.068 | 0.065 | 0.053 | 0.052 | 0.052 | 0.053 |
| 1:9 | 0.202 | 0.197 | 0.228 | 0.274 | 0.292 | 0.308 |
| 1:27 | 0.697 | 0.780 | 0.752 | 0.786 | 0.771 | 0.871 |
| 1:81 | 0.996 | 0.991 | 0.903 | 1.030 | 0.971 | 0.966 |
| 1:243 | 1.082 | 1.047 | 1.030 | 1.135 | 1.050 | 1.073 |
| 1:729 | 0.964 | 0.988 | 0.950 | 0.978 | 0.918 | 0.964 |
| 0 | 0.935 | 0.960 | 0.932 | 0.898 | 0.896 | 0.956 |

IL4-N-his

| | | | |
|---|---|---|---|
| Second antibody | Mouse anti-His, HRP (1:4000) | | |
| EC₅₀(nM) | 1.150 | 1.328 | 1.546 |

### 2. Binding Activity of Murine Antibody 18H7 and Humanized Antibodies 18H7 H1L1, 18H7 H2L2, 18H7 H2L3, 18H7 H3L2 and 18H7 H3L3 to Antigens IL-4RA-hFc

### 2.1 The Binding Activity of the Antibodies 18H7, 18H7 H1L1, 18H7 H2L2, 18H7 H2L3, 18H7 H3L2 and 18H7 H3L3 to the Antigens Human IL4RA-hFc or Human IL4RA-mFc was Measured by Indirect ELISA.

Procedures: The ELISA plates were coated with human IL4RA-hFc or human IL4RA-mFc and were incubated, and target antibodies were added after blocking. Goat anti-mouse IgG (H+L) and HRP (purchased from Jackson ImmunoResearch Inc., Cat No. 109-035-062), or goat anti-human IgG and HRP (purchased from Jackson ImmunoResearch Inc., Cat No. 109-035-088) were added. The plates were incubated and washed before TMB (Neogen, 308177) was added for chromogenic reaction. At the end of the reaction, the absorbance at 450 nm was measured by a plate reader. The data were analyzed by SoftMax Pro 6.2.1.

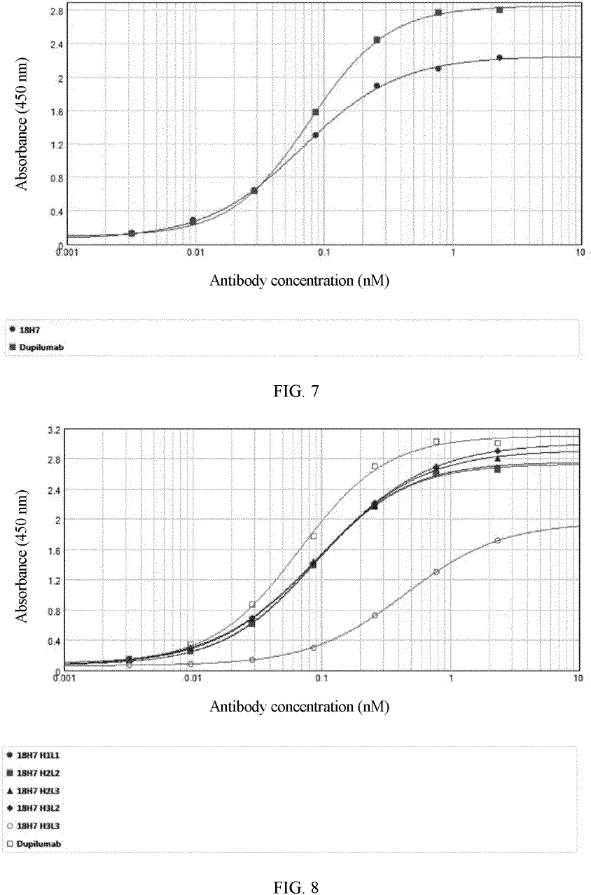

The readings at 450 nm show that antibodies 18H7, 18H7 H1L1, 18H7 H2L2, 18H7 H2L3, 18H7 H3L2 and 18H7 H3L3 are all capable of effectively binding to antigens human IL4RA-hFc or human IL4RA-mFc in a dose-dependent manner. By 4-parameter logistic regression of absorbance vs. antibody concentration, as shown in Tables 7 and 8, 18H7, 18H7 H1L1, 18H7 H2L2, 18H7 H2L3, 18H7 H3L2 and 18H7 H3L3 are all capable of binding to antigens human IL4RA-hFc or human IL4RA-mFc effectively, and antibodies 18H7 H1L1, 18H7 H2L2 and 18H7 H2L3 demonstrate comparable binding activities to that of the reference antibody dupilumab against the same target (FIGS. 7 and 8).

TABLE 7

Binding activity of 18H7 and dupilumab to antigens human IL4RA-hFc or human IL-4 RA-mFc

| Antibody dilution (µg/mL) | Antigen (1 µg/mL) | | | |
|---|---|---|---|---|
| | IL-4RA-hFc | | IL-4RA-mFc | |
| | 18H7 | | Dupilumab | |
| 0.333 | 2.239 | 2.219 | 2.758 | 2.858 |
| 1:3 | 2.065 | 2.128 | 2.748 | 2.801 |
| 1:9 | 1.913 | 1.867 | 2.388 | 2.500 |
| 1:27 | 1.278 | 1.333 | 1.469 | 1.679 |
| 1:81 | 0.640 | 0.650 | 0.599 | 0.673 |
| 1:243 | 0.276 | 0.302 | 0.250 | 0.281 |
| 1:729 | 0.132 | 0.137 | 0.126 | 0.137 |
| 0 | 0.051 | 0.053 | 0.076 | 0.069 |

| | | |
|---|---|---|
| Second antibody | Goat anti-mouse IgG (H+L), HRP (1:5000) | Goat anti-human IgG (H+L), HRP (1:5000) |
| EC₅₀ (nM) | 0.068 | 0.077 |

TABLE 8

Binding activity of 18H7 H1L1, 18H7 H2L2, 18H7 H2L3, 18H7 H3L2, 18H7 H3L3 and dupilumab to antigen IL4RA-mFc

| Antibody dilution (µg/mL) | Antigen: IL-4RA-mFc | | | | | | | | | | | |
|---|---|---|---|---|---|---|---|---|---|---|---|---|
| | 18H7 H1L1 | | 18H7 H2L2 | | 18H7 H2L3 | | 18H7 H3L2 | | 18H7 H3L3 | | Dupilumab | |
| 0.333 | 2.683 | 2.666 | 2.580 | 2.720 | 2.750 | 2.848 | 2.862 | 2.943 | 1.729 | 1.704 | 2.972 | 3.042 |
| 1:3 | 2.630 | 2.616 | 2.611 | 2.595 | 2.726 | 2.675 | 2.696 | 2.673 | 1.314 | 1.279 | 2.989 | 3.057 |
| 1:9 | 2.226 | 2.101 | 2.153 | 2.174 | 2.150 | 2.185 | 2.197 | 2.235 | 0.719 | 0.744 | 2.654 | 2.740 |
| 1:27 | 1.441 | 1.373 | 1.401 | 1.382 | 1.439 | 1.437 | 1.429 | 1.410 | 0.291 | 0.308 | 1.743 | 1.815 |
| 1:81 | 0.682 | 0.575 | 0.626 | 0.613 | 0.677 | 0.679 | 0.685 | 0.708 | 0.139 | 0.134 | 0.847 | 0.896 |
| 1:243 | 0.282 | 0.223 | 0.257 | 0.253 | 0.288 | 0.292 | 0.295 | 0.300 | 0.085 | 0.086 | 0.329 | 0.358 |
| 1:729 | 0.133 | 0.115 | 0.127 | 0.124 | 0.138 | 0.143 | 0.146 | 0.150 | 0.078 | 0.068 | 0.147 | 0.165 |
| 0 | 0.059 | 0.062 | 0.060 | 0.062 | 0.059 | 0.063 | 0.062 | 0.059 | 0.065 | 0.063 | 0.068 | 0.063 |

| | | | | | | |
|---|---|---|---|---|---|---|
| Second antibody | Goat anti-human IgG (H+L), HRP (1:5000) | | | | | |
| EC₅₀ (nM) | 0.087 | 0.087 | 0.094 | 0.100 | 0.446 | 0.067 |

### 2.2 Activity of Antibodies 18H7, 18H7 H1L1, 18H7 H2L2, 18H7 H2L3 and 18H7 H3L2 for Blocking the Binding Between IL4-N-his and the Antigen IL4RA-hFc by Competitive ELISA

The EC₅₀ (median effect concentration) of 18H7, 18H7 H1L1, 18H7 H2L2, 18H7 H2L3 and 18H7 H3L2 for competing with ligand human IL4-N-His for binding to the antigen human IL4RA-hFc was assayed by ELISA, so as to investigate the activity of 18H7, 18H7 H1L1, 18H7 H2L2, 18H7 H2L3 and 18H7 H3L2 for blocking the binding of the ligand to the target antigen human IL4RA-hFc.

The ELISA plates were coated with human IL4RA-hFc and blocked before the antibodies were added. An equal volume of human IL4-N-His (synthesized by Akeso, Inc., an IL4 connected with 6 His tags at the N terminus) was added, and the mixture was well mixed and incubated. After the plates were washed, mouse anti-His, HRP (purchased from CoWin Biosciences, Cat No. CW0285M) was added for incubation. The plates were subjected to another wash. Chromogenic reaction was started by adding TMB (Neogen, 308177) and was then terminated. At the end of the reaction, the OD value at 450 nm wavelength was measured by a plate reader, and was analyzed and processed by SoftMax Pro 6.2.1. The results are shown in Tables 9 and 10.

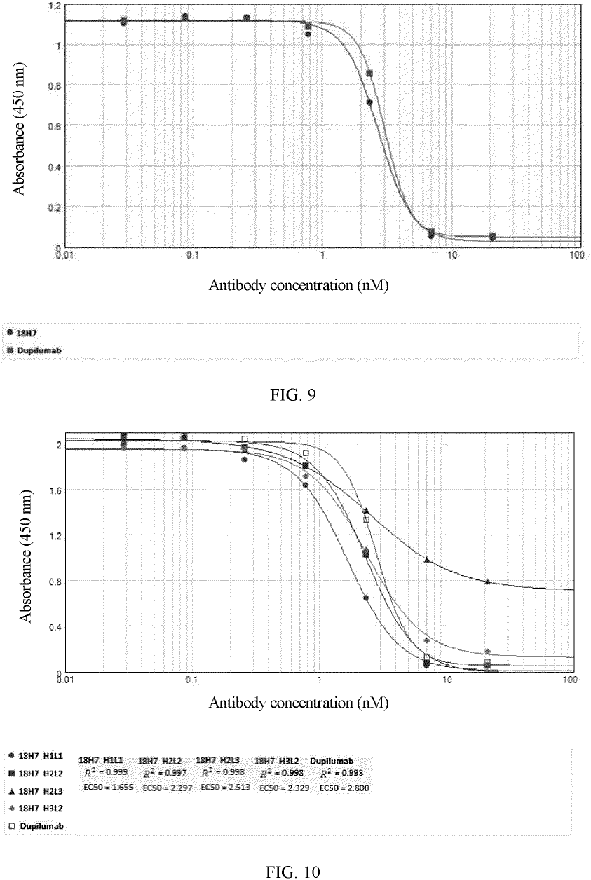

By 4-parameter logistic regression of absorbance vs. antibody concentration, the blocking EC₅₀ of the antibodies was calculated. As shown in FIGS. 9 and 10, 18H7, 18H7 H1L1, 18H7 H2L2 and 18H7 H3L2 are all capable of effectively blocking the binding of the ligand human IL4-N-His to the antigen human IL4RA-hFc in a dose-dependent manner. Antibodies 18H7 H1L1, 18H7 H2L2 and 18H7 H3L2 demonstrate superior activities for competing with human IL4-N-His for binding to human IL4RA-hFc as compared to that of the reference antibody dupilumab for the same antigen.

TABLE 9

Activity assay of 18H7 and dupilumab in competing with human IL4-N-His for binding to human IL4RA-hFc

| Antibody dilution (µg/mL) | Antigen: IL4RA-hFc (2 µg/mL) | | | |
|---|---|---|---|---|
| | 18H7 | | Dupilumab | |
| 3 | 0.046 | 0.045 | 0.054 | 0.052 |
| 1:3 | 0.051 | 0.053 | 0.074 | 0.076 |
| 1:9 | 0.704 | 0.722 | 0.837 | 0.877 |
| 1:27 | 1.047 | 1.055 | 1.056 | 1.123 |
| 1:81 | 1.117 | 1.144 | 1.100 | 1.149 |
| 1:243 | 1.113 | 1.162 | 1.126 | 1.126 |
| 1:729 | 1.097 | 1.115 | 1.076 | 1.158 |
| 0 | 1.157 | 1.127 | 1.123 | 1.093 |

IL-4-N-his:0.15 µg/ml

| | | |
|---|---|---|
| Second antibody | Mouse anti-His, HRP (1:4000) | |
| EC₅₀ (nM) | 2.714 | 3.033 |

TABLE 10

Activity assay of 18H7 H1L1, 18H7 H2L2, 18H7 H2L3, 18H7 H3L2 and dupilumab in competing with human IL4-N-His for binding to human IL4RA-hFc

| Antibody dilution (µg/mL) | Antigen: IL4RA-hFc (2 µg/mL) | | | | | | | | | |
|---|---|---|---|---|---|---|---|---|---|---|
| | 18H7 H1L1 | | 18H7 H2L2 | | 18H7 H2L3 | | 18H7 H3L2 | | Dupilumab | |
| 3 | 0.053 | 0.051 | 0.057 | 0.061 | 0.781 | 0.806 | 0.183 | 0.184 | 0.086 | 0.079 |
| 1:3 | 0.051 | 0.054 | 0.074 | 0.083 | 0.956 | 1.011 | 0.280 | 0.279 | 0.127 | 0.126 |
| 1:9 | 0.564 | 0.735 | 0.985 | 1.072 | 1.439 | 1.388 | 1.036 | 1.100 | 1.326 | 1.341 |
| 1:27 | 1.599 | 1.671 | 1.835 | 1.785 | 1.840 | 1.775 | 1.761 | 1.673 | 1.887 | 1.947 |
| 1:81 | 1.839 | 1.884 | 1.939 | 2.009 | 1.971 | 1.926 | 1.947 | 1.964 | 2.029 | 2.059 |
| 1:243 | 1.938 | 1.993 | 2.037 | 2.055 | 2.086 | 2.057 | 1.960 | 1.946 | 2.079 | 2.049 |
| 1:729 | 1.979 | 1.995 | 2.101 | 2.043 | 2.048 | 2.002 | 1.975 | 1.948 | 2.039 | 2.029 |
| 0 | 1.919 | 1.991 | 2.063 | 2.024 | 2.096 | 1.973 | 1.928 | 1.938 | 2.034 | 1.975 |

IL-4-N-his:0.15 µg/ml

| | | | | | |
|---|---|---|---|---|---|
| Second antibody | Mouse anti-His, HRP (1:4000) | | | | |
| EC₅₀ (nM) | 1.655 | 2.297 | 2.513 | 2.329 | 2.800 |

### 3. Binding Activity of Humanized Antibodies 20G10 H3L3 and 13E5 H4L4 to Antigen IL-4RA-hFc

### 3.1 The Activity of Antibodies 20G10 H3L3 and 13E5 H4L4 for Blocking The Binding of IL4-N-His to the Antigen IL4RA-hFc was Measured by Competitive ELISA

The EC₅₀ (median effect concentration) of 20G10 H3L3 and 13E5 H4L4 for competing with ligand human IL4-N-His for binding to the antigen human IL4RA-hFc was assayed by ELISA, so as to investigate the activity of 20G10 H3L3 and 13E5 H4L4 for blocking the binding of the ligand to the target antigen human IL4RA-hFc.

The ELISA plates were coated with human IL4RA-hFc and blocked before the antibodies were added. An equal volume of human IL4-N-His (synthesized by Akeso, Inc.) was added, and the mixture was well mixed and incubated. After the plates were washed, mouse anti-His, HRP (purchased from CoWin Biosciences, Cat No. CW0285A) was added for incubation. The plates were subjected to another wash. Chromogenic reaction was started by adding TMB (Neogen, 308177) and was then terminated. At the end of the reaction, the OD value at 450 nm wavelength was measured by a plate reader, and was analyzed and processed by SoftMax Pro 6.2.1. The results are shown in Table 11.

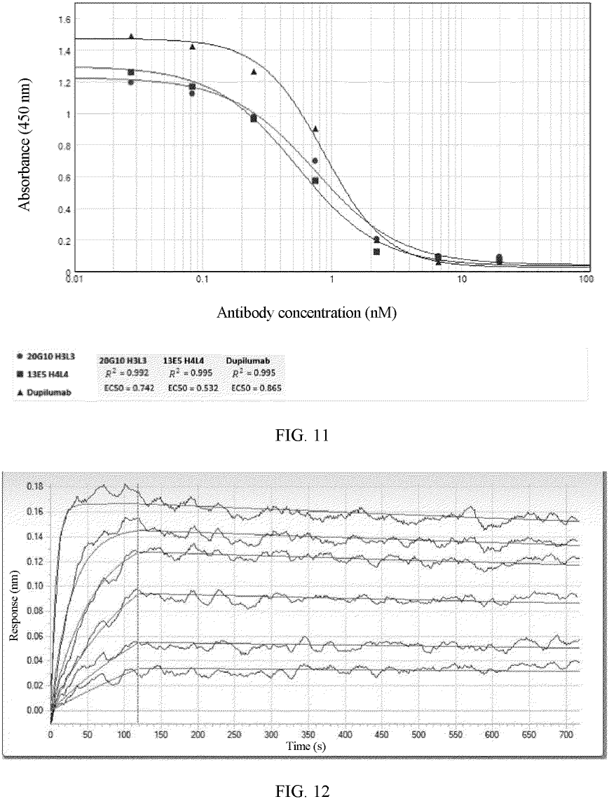

By 4-parameter logistic regression of absorbance vs. antibody concentration, the blocking EC₅₀ of the antibodies was calculated. As shown in FIG. 11, 20G10 H3L3 and 13E5 H4L4 are all capable of effectively blocking the binding of the ligand human IL4-N-His to the antigen human IL4RA-hFc in a dose-dependent manner. The antibodies 20G10 H3L3 and 13E5 H4L4 demonstrate superior activities for competing with human IL4-N-His for binding to human IL4RA-hFc as compared to that of the reference antibody dupilumab for the same antigen.

TABLE 11

Activity assay of 20G10 H3L3, 13E5 H4L4 and dupilumab in competing with human IL4-N-His for binding to human IL4RA-hFc

| Antibody dilution (µg/mL) | Antigen: IL4RA-hFc (2 µg/mL) | | | | | |
|---|---|---|---|---|---|---|
| | 20G10 H3L3 | | 13E5 H4L4 | | Dupilumab | |
| 3 | 0.093 | 0.082 | 0.065 | 0.074 | 0.059 | 0.059 |
| 1:3 | 0.099 | 0.094 | 0.097 | 0.071 | 0.059 | 0.060 |
| 1:9 | 0.199 | 0.212 | 0.123 | 0.119 | 0.201 | 0.197 |
| 1:27 | 0.701 | 0.693 | 0.554 | 0.594 | 0.853 | 0.948 |
| 1:81 | 0.948 | 1.001 | 0.944 | 0.976 | 1.241 | 1.287 |
| 1:243 | 1.118 | 1.131 | 1.137 | 1.200 | 1.443 | 1.400 |
| 1:729 | 1.210 | 1.180 | 1.252 | 1.269 | 1.534 | 1.441 |
| 0 | 1.295 | 1.274 | 1.328 | 1.349 | 1.559 | 1.482 |

IL4-N-his:0.15 µg/ml

| | | | |
|---|---|---|---|
| Second antibody | Mouse anti-His, HRP (1:4000) | | |
| EC50(nM) | 0.742 | 0.532 | 0.865 |

### Example 8. Affinity Constant Assay of Humanized Antibodies 13E5 H1L1, 13E5 H2L2, 13E5 H3L3, 13E5 H4L2, 13E5 H4L4, 18H7 H1L1, 18H7 H2L2 and 18H7 H3L2 for Human IL-4RA

By Fortebio molecular interaction instrument, the affinity constant of humanized antibodies 13E5H 1L1, 13E5H 2L2, 13E5 H3L3, 13E5H 4L2, 13E5H 4L4, 18H7 H1L1, 18H7 H2L2, 18H7 H3L2 and dupilumab for binding human IL-4RA were measured.

5 µg/mL antigen was immobilized on AMC sensor by 60 s of incubation. The sensor was then equilibrated in PBST for 300 s and incubated for 120 s in the antibodies at 0.39-25 nM (two-fold serial dilution) for binding the antibodies to the antigen immobilized on the sensor. The remaining antibodies were dissociated with the antigen by a 600-second incubation in PBST. The sensor was refreshed in 10 mM glycine, pH 1.7. The data were analyzed by 1:1 model fitting to obtain affinity constants.

The affinity constants of humanized antibodies 13E5 H4L4, 18H7 H1L1 and dupilumab (reference) for human IL4RA are shown in Table 12 and FIGS. 12-14.

TABLE 12

Affinity constant assay of 13E5 H4L4, 18H7 H1L1 and dupilumab for human IL4RA

| Name | K_{D} (M) | kon(⅟Ms) | kon Error | kdis(⅟s) | kdis Error | Rmax(nm) |
|---|---|---|---|---|---|---|
| 13E5 H4L4 | 2.63E-11 | 5.56E+06 | 1.60E+05 | 1.46E-04 | 9.74E-06 | 0.1370-0.1667 |
| 18H7 H1L1 | 3.09E-11 | 4.97E+06 | 1.94E+05 | 1.54E-04 | 1.33E-05 | 0.1026-0.1445 |
| Dupilumab | 1.14E-11 | 7.08E+06 | 2.15E+05 | 8.10E-05 | 1.13E-05 | 0.1325-0.2184 |

K_{D} is the affinity constant; K_{D} = kdis/kon.

The results show that the humanized antibodies 13E5 H4L4, 18H7 H1L1 and human IL4RA have high binding capacities to the target antigen, which are comparable to that of the reference antibody dupilumab.

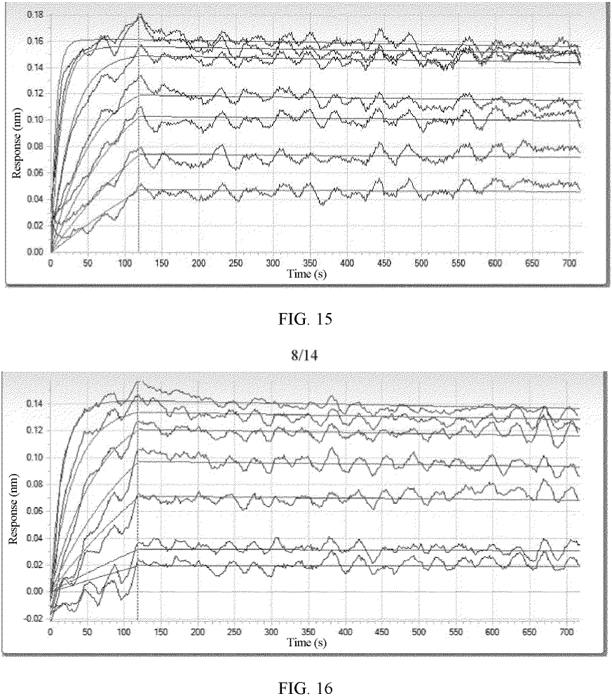

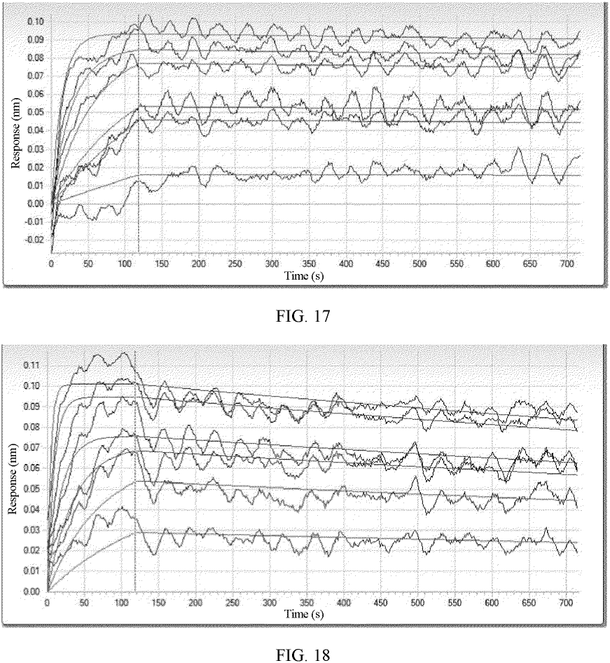

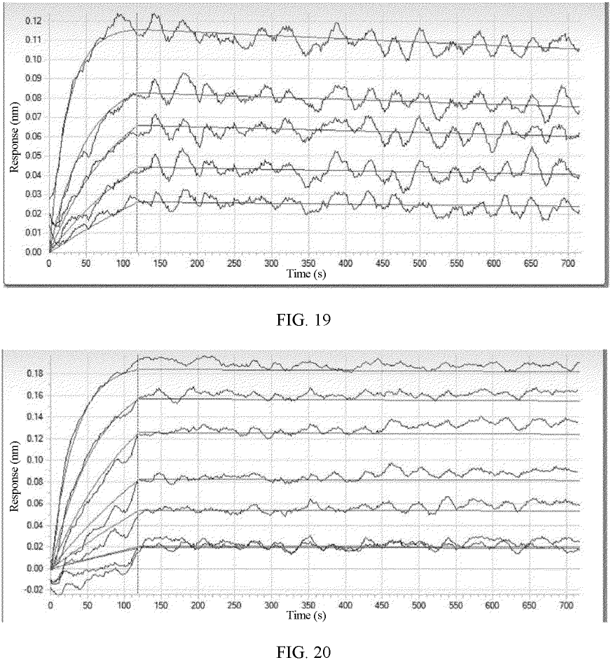

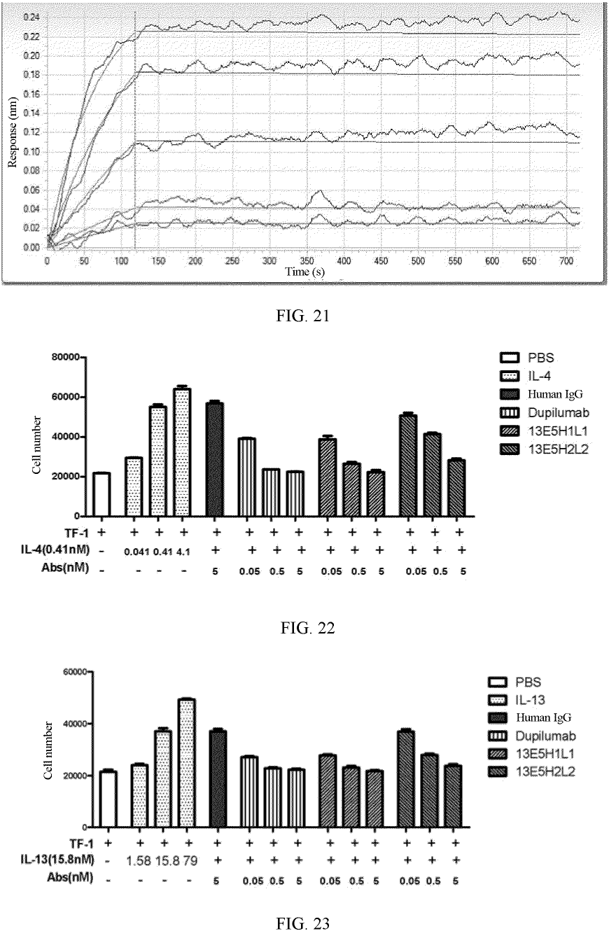

The affinity constants of humanized antibodies 13E5 H1L1, 13E5 H2L2, 13E5 H3L3, 13E5 H4L2, 18H7 H2L2, 18H7 H3L2 and dupilumab (reference) for human IL-4RA are shown in Table 13 and FIGS. 15-21.

TABLE 13

Affinity constant assay of 13E5 H1L1, 13E5 H2L2, 13E5 H3L3, 13E5 H4L2, 18H7 H2L2, 18H7 H3L2 and dupilumab for human IL4RA

| Antibody | K_{D} (M) | kon(⅟Ms) | kon Error | kdis(⅟s) | kdis Error | Rmax(nm) |
|---|---|---|---|---|---|---|
| 13E5 H1L1 | 1.00E-11 | 5.78E+06 | 1.94E+05 | 5.79E-05 | 1.12E-05 | 0.1347-0.2008 |
| 13E5 H2L2 | 2.83E-11 | 2.44E+06 | 9.77E+04 | 6.90E-05 | 1.44E-05 | 0.1377-0.1944 |
| 13E5 H3L3 | 1.82E-11 | 2.79E+06 | 1.46E+05 | 5.09E-05 | 1.90E-05 | 0.0824-0.1332 |
| 13E5 H4L2 | 1.79E-11 | 1.78E+07 | 1.16E+06 | 3.18E-04 | 2.15E-05 | 0.0521-0.1012 |
| 18H7 H2L2 | 2.39E-11 | 6.47E+06 | 2.63E+05 | 1.54E-04 | 1.60E-05 | 0.0913-0.117 |
| 18H7 H3L2 | 1.71E-11 | 1.27E+06 | 5.38E+04 | 2.16E-05 | 1.44E-05 | 0.1731-0.3601 |
| Dupilumab | 1.17E-11 | 2.35E+06 | 1.68E+05 | 2.75E-05 | 1.73E-05 | 0.2131-0.3131 |

K_{D} is the affinity constant; K_{D} = kdis/kon.

The result shows that the affinity of the humanized antibody 13E5 H1L1 for the antigen is superior to that of the reference antibody dupilumab; the dissociation rate constants kdis of 13E5 H1L1, 13E5 H2L2, 18H7 H2L2 and 18H7 H3L2 with human IL-4RA were less than that of the reference antibody dupilumab, suggesting more stable binding of 13E5 H2L2, 18H7 H2L2 and 18H7 H3L2 to human IL-4RA. The association rate constants of 13E5 H1L1 and 13E5 H4L2 with the antigen human IL-4RA are greater than that of the reference antibody dupilumab, suggesting faster binding of 13E5 H1L1 and 13E5 H4L2 to the antigen human IL-4RA than dupilumab.

### Example 9. Cellular Bioactivity Assay

Cellular bioactivity of 13E5 H1L1, 13E5 H2L2, 13E5 H4L2, 13E5 H4L4, 18H7 H1L1 and 18H7 H2L2 for inhibiting human IL-4/IL-13-induced TF-1 cell proliferation was analyzed. The procedures are as follows:

TF-1 cells (purchased from American Type Culture Collection, Cat. No. CRL-2003) were cultivated (complete medium: RPMI 1640 + 10% FBS + 2.5 g/L glucose + 2 ng/mL GM-CSF). On the day of assay, TF-1 cells were separated by centrifugation, resuspended in a medium free of GM-CSF, and counted. The cells were inoculated into 96-well plates at 20,000 cells/well. The treatment was given as designed: the final concentrations of the antibodies were 0.05, 0.5 and 5 nM; three concentrations 0.041, 0.41 and 4.1 nM were set for IL-4, and the antibody group employed 0.41 nM IL-4; three concentrations 1.58, 15.8 and 79 nM were set for IL-13, and the antibody panel employed 15.8 nM IL-13. After administration, the 96-well plates were incubated in a 5% carbon dioxide incubator at 37° C. for 72 h. After the 72 hours, CCK8 reagent was added according to the instruction of CCK8 kit (purchased from Dojindo Laboratories, Japan, Cat No. CK04). The mixture was mixed well, incubated at 37° C. for 3-4 hours in a 5% carbon dioxide incubator, and the OD value at 450 nm was read. A curve of OD vs. cell number was plotted by seeding serially diluted TF-1 cells into 96-well plates, adding CCK8 reagent, incubating the plates in a 5% carbon dioxide incubator at 37° C. for 3-4 h and reading the OD value at 450 nm. The cell numbers of the groups were calculated according to the OD value, and GraphPad Prism 5 were used for plotting.

The isotype reference antibody was human anti-hen egg lysozyme IgG (anti-HEL) derived from the variable region of the Fab F10.6.6 sequence in Affinity Maturation Increases the Stability and Plasticity of the Fv Domain of Anti-Protein Antibodies (Acierno et al., J Mol Biol., 2007, 374(1):130-46). The isotype was synthesized by Akeso, Inc.

Nanjing Genscript Biology was entrusted for codon optimization and gene synthesis for the heavy and light chain (complete sequence or variable region) genes of the human IgG antibodies. With reference to the Molecular Cloning: A Laboratory Manual (Second Edition), the heavy and light chain genes were subcloned into the heavy and antibody light chain expression vectors (both were pcDNA3.1 vector) for mammalian expression system by standard molecular cloning techniques such as PCR, enzymatic digestion, DNA gel electrophoresis, ligation and transformation, colony PCR or digestion and identification. The heavy and light chain genes of the recombinant expression vector were further sequenced and analyzed (the heavy and light chain sequences are set forth in SEQ ID NOs: 122 and 123). After the sequence was verified to be correct, endotoxin-free expression plasmids were prepared in a large scale, and the heavy and light chain plasmids were transiently co-transfected into HEK293 cells for expression of recombinant antibody. After 7 days of culture, the cell culture medium was collected and affinity purified using a Protein A column (GE), and the quality of the resulting antibody sample was determined using SDS-PAGE and SEC-HPLC standard analysis techniques.

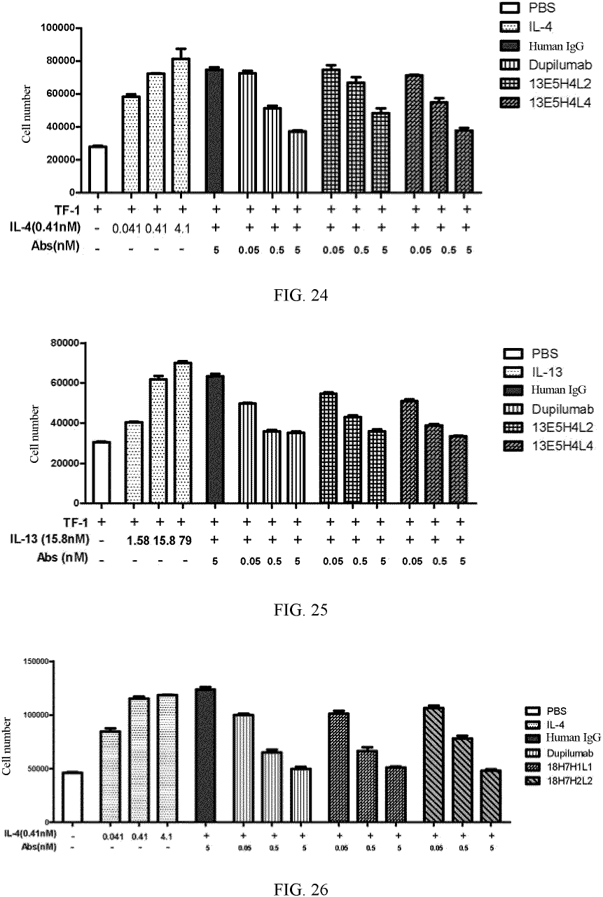

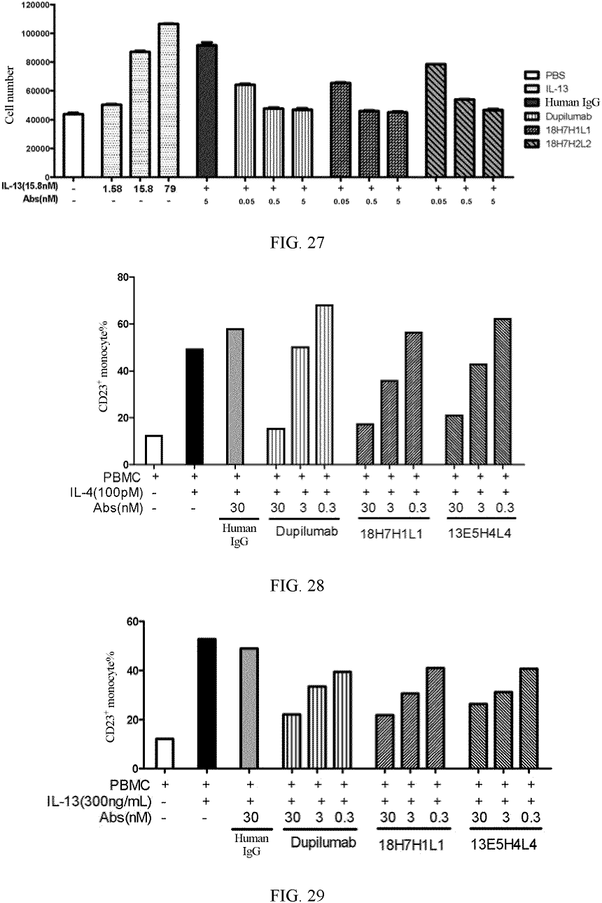

The results are shown in FIGS. 22-27. As can be seen in FIGS. 22-27, both human IL-4 and human IL-13 are able to effectively promote the proliferation of TF-1 cells in a dose-dependent manner; as compared to isotype reference antibodies (human IgG), dupilumab, 13E5 H1L1, 13E5 H2L2, 13E5 H4L2, 13E5 H4L4, 18H7 H1L1 and 18H7 H2L2 can specifically inhibit TF-1 cell proliferation induced by IL-4 and IL-13 in a dose-dependent manner.

The results show that 13E5 H1L1, 13E5 H2L2, 13E5 H4L2, 13E5 H4L4, 18H7 H1L1 and 18H7 H2L2 can specifically inhibit the proliferation of TF-1 cells induced by human IL-4 and human IL-13; antibodies 13E5 H1L1, 13E5 H4L4 and 18H7 H1L1 demonstrate comparable activities to reference antibody dupilumab.

### Example 10. 13E5H4L4 and 18H7H1L1 Inhibiting Up-Regulation of CD23 Expression in PBMCs

This example was intended to examine the bioactivity of 13E5 H4L4 and 18H7 H1L1 for neutralizing the CD23 expression up-regulation on human PBMC surface induced by human IL-4 and human IL-13 through flow cytometry. The procedures are as follows:

Normal human peripheral blood (with heparin) was separated by Ficoll density gradient centrifugation to give human fresh PBMCs. After 3 centrifugations and washes, the cells were counted and the density was adjusted to 2.5 × 10⁶ cells/mL. The cells were seeded into low attachment 96-well plates at 200 µL of PBMCs per well (i.e., 500,000 cells/well); 25 µL of the antibodies dupilumab, 13E5 H4L4 or 18H7 H1L1 (at final concentrations of 30, 3 and 0.3 nM, respectively) were added to each well, and blank reference and isotype reference human IgG (at a final concentration of 30 nM) were set. The cells were incubated at room temperature for 30 min. After 30 min, 25 µL of human IL-4 (final concentration of 100 pM) or 25 µL of human IL-13 (final concentration of 300 ng/mL) was added and the system was incubated for 2.5 days. After the 2.5 days, the PBMCs were separated and transferred into 1.5-mL EP tubes, added with 500 µL of 1% PBSA and centrifuged at 1000× g for 5 min. The supernatant was discarded, and the residues were added with 50 µL of CD23-PE antibody (50-fold diluted with 1% PBSA) and incubated on ice for 40 min. After the 40-min incubation, the system was added with 1 mL of 1% PBSA and centrifuged at 1000× g for 5 min before the supernatant was discarded. The cells were resuspended in 200 µL of 1% PBSA, and transferred into a flow cytometry tube for assay.

The results are shown in FIGS. 28 and 29.

The results show that human IL-4 and human IL-13 can up-regulate the expression level of CD23 on the surface of human PBMCs, and 13E5 H4L4 and 18H7 H1L1 can specifically bind to human IL-4RA, thereby effectively blocking the up-regulation of the CD23 expression level by IL-4 and IL-13.

### Example 11. Effect of 13E5 H4L4 on B-hIL4Ra Mouse Model with HDM-Induced Skin Inflammation

The procedures are as follows:

A B-hIL4Ra mouse (C57BL/6 background) skin inflammation model was established by inducing with HDM (human dust mite allergen, Greer Laborct, Lot No. 348717). The mice were divided into 6 groups with 7 mice in each, including isotype reference group (anti-HEL), dupilumab high-dose group (80 mg/kg; commercially available dupilumab from Sanofi and Regeneron Pharmaceuticals) and low-dose group (20 mg/kg; commercially available as Dupilumab from Sanofi and Regeneron Pharmaceuticals), 13E5 H4L4 high-dose group (80 mg/kg) and low-dose group (20 mg/kg). The treatment was given by subcutaneous injection on D-1, D2, D5 and D8. In the normal group, the animal model was established by daily intradermal injection of normal saline. In the isotype reference group, the animal model was established with daily intradermal injection of HDM (37.5 µg × 2/25 µL/day) for 7 days and daily intradermal injection of HDM (25 µg × 2/25 µL/day)for 3 days. In the antibody treatment groups, the animal model was established with daily intradermal injection of HDM (37.5 µg × 2/25 µL/day)for 7 days and daily intradermal injection of HDM (25 µg × 2/25 µL/day)for 3 days. Epidermal thickness of the mice was measured to investigate the effect of 13E5 H4L4 in inhibiting the epidermal thickness in B-hIL4Ra mouse skin inflammation model.

TABLE 14

Dosage and regimen

| Group | n | Animal model | Treatment |
|---|---|---|---|
| Normal group | 7 | Normal saline, 25 µL/day on D0-D9, intradermal injection, daily | Normal saline, subcutaneous injection, D-1, D2, D5 and D8 |
| hIgG4 (i.e., anti-HEL) | 7 | HDM, 37.5 µg × 2/25 µL/day, D0-D6; 25 µg × 2/25 µL/day, D7-D9, daily intradermal injection | Anti-HEL, 80 mg/kg, subcutaneous injection, D-1, D2, D5 and D8 |
| Dupilumab 80 mg/kg | 7 | | Dupilumab, 80 mg/kg, subcutaneous injection, D-1, D2, D5 and D8 |
| Dupilumab, 20 mg/kg | 7 | | Dupilumab, 80 mg/kg, subcutaneous injection, D-1, D2, D5 and D8 |
| 13E5 H4L4, 80 mg/kg | 7 | | 13E5 H4L4, 80 mg/kg, subcutaneous injection, D-1, D2, D5 and D8 |
| 13E5 H4L4, 20 mg/kg | 7 | | 13E5 H4L4, 20 mg/kg, subcutaneous injection, D-1, D2, D5 and D8 |

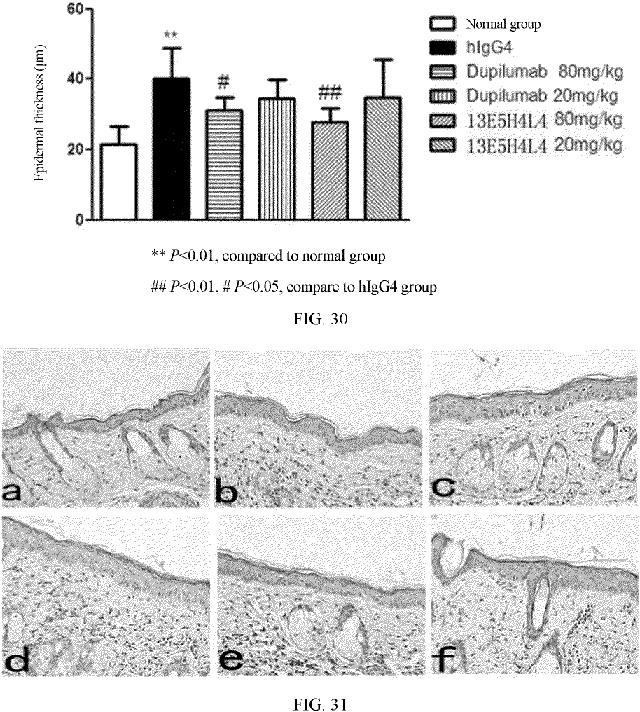

Note: D0 is the first day of model establishment. The normal saline and HDM were administered at the upper right back and lower right back respectively. The results are shown in FIG. 30. As compared with the isotype reference antibody hIgG4, 13E5 H4L4 of different doses can effectively inhibit the thickening of epidermis, and the blood concentration of 13E5 H4L4 is higher than that of dupilumab at 80 mg/kg, which corresponds to the efficacy.

The pathological sections are shown in FIG. 31: in the B-hIL4Ra mouse skin inflammation model established by 37.5 µg + 25 µg HDM intradermal injection, the thickness of epidermis in the model group is significantly thickened as compared with that of the normal group, the thickening of epidermis in mice can be significantly inhibited by dupilumab 80 mg/kg and 13E5 H4L4 80 mg/kg subcutaneous injections with good dose-response relationship. 13E5 H4L4 80 mg/kg is slightly superior to dupilumab 80 mg/kg.

The preferred embodiments of the present invention have been described above in detail, but the present invention is not limited to the embodiments. Those skilled in the art can make various equivalent modifications or replacements without violating the spirit of the present invention. These equivalent modifications or replacements are included in the scope defined by the claims of the present application.

### SEQUENCE LISTING

13E5 heavy chain variable region

GAGGTGCAGCTGCAGCAGAGCGGACCAGAGCTGGTGAAGCCTGGCGCCTC
TGTGAAGATCAGCTGTAAGACCTCCGGCTACACCTTCACAGAGTATACAA
TCCACTGGGTGAAGCAGAACCACGGCAAGAGCCTGGAGTGGATCGGCGGC
ATCAATCCTAACAATGGCGGCACCGTGTACAACCAGAACTTCAAGGGCAA
GGCCACCCTGACAGTGGACAAGAGCTCCTCTACCGCCTATATGGAGCTGA
GGTCTCTGACAAGCGAGGACTCCGCCGTGTACTATTGCGCCAGAGTGCGG
AGAGGCATGGATTACTGGGGCCAGGGCACCTCCGTGACAGTGAGCTCC (
SEQ ID NO: 1)

EVQLQQSGPELVKPGASVKISCKTSGYTFTEYTIHWVKQNHGKSLEWIGG INPNNGGTVYNQNFKGKATLVDKSSSTAYMELRSLTSEDSAVYYCARVRR GMDYWGQGTSVTVSS (SEQ ID NO: 2)

13E5 light chain variable region

GACATCGTGATGACCCAGTCCCACAAGTTTATGTCCACATCTGTGGGCGA
CAGGGTGTCCATCACCTGTAAGGCCTCTCAGGATGTGACCACAGCCGTGG
CCTGGTACCAGCAGAAGCCAGGCCAGTCTCCCAAGCTGCTGATCTATAGC
GCCTCCTACCGGTATACAGGCGTGCCCGACAGATTCACCGGCTCTGGCAG
CGGCACAGATTTCACCTTTACAATCAGCTCCGTGCAGGCAGAGGACCTGG
CCGTGTACTATTGCCAGCAGCACTACTCTGCCCCTTGGACCTTCGGCGGA
GGAACAAACCTGGAGATCAAG (SEQ ID NO: 3)

DIVMTQSHKFMSTSVGDRVSITCKASQDVTTAVAWYQQKPGQSPKLLIYS ASYRYTGVPDRFTGSGSGTDFTFTISSVQAEDLAVYYCQQHYSAPWTFGG
GTNLEIK (SEQ ID NO: 4)

18H7 heavy chain variable region

GATGTGAAGCTGGTGGAGTCCGGCGGCGACCTGGTGAACCTGGGCGGAAG
CCTGAAGCTGAGCTGTGCCGCCAGCGGCTTCACATTTTCCTCCAGCTACA
TGAGCTGGGTGAGGCAGACACCCGAGAGGAGACTGGAGCTGGTGGCCGCC
ATCAACAGCAATGGCGGCAAGACATACTACCCTGATACCGTGAAGGGCAG
ATTCACCATCAGCAGGGACAACGCCAAGAACACCCTGTACCTGCAGATGT
CCGGCCTGAAGACAGAGGACACAGCCCTGTACTACTGCACCAGGCAGAGG
GGCAACTACGTGGGCGCCATGGACTACTGGGGCCAGGGCACAAGCGTGAC
CGTGAGCTCC (SEQ ID NO: 5)

DVKLVESGGDLVNLGGSLKLSCAASGFTFSSSYMSWVRQTPERRLELVAA INSNGGKTYYPDTVKGRFTISRDNAKNTLYLQMSGLKTEDTALYYCTROR GNYVGAMDYWGQGTSVTVSS (SEQ ID NO: 6)

18H7 light chain variable region

GACATCGTGATGACACAGTCCCACAAGTTTATGTCCACATCCGTGGGCGA
TAGGGTGTCCATCACATGCAAGGCCAGCCAGGACGTGAGCACAGCCGTGG
TGTGGTACCAGCAGAAGCCTGGCCAGAGCCCCACCCTGCTGATCTACTCC
GCCAGCTACAGGTACACAGGCGTGCCTGACAGATTCACAGGCTCCGGCAG
CGGCACCGATTTCACCTTTACCATCTCCTCCGTGCAGGCCGAGGATCTGG
CCGTGTACTACTGCCACCAGTACTACGGCAGCCCCCCCACATTCGGCGGC
GGAACAAAGCTGGAGATCAAG (SEQ ID NO: 7)

DIVMTQSHKFMSTSVGDRVSITCKASQDVSTAVVWYQQKPGQSPTLLIYS ASYRYTGVPDRFTGSGSGTDFTFTISSVQAEDLAVYYCHOYYGSPPTFGG
GTKLEIK (SEQ ID NO: 8)

13E5 CDR

HCDR1: GYTFTEYT (SEQ ID NO: 9)

HCDR2: INPNNGGT (SEQ ID NO: 10)

HCDR3: ARVRRGMDY (SEQ ID NO: 11)

LCDR1: QDVTTA (SEQ ID NO: 12)

LCDR2: SAS (SEQ ID NO: 13)

LCDR3: QQHYSAPWT (SEQ ID NO: 14)

18H7 CDR

HCDR1: GFTFSSSY (SEQ ID NO: 15)

HCDR2: INSNGGKT (SEQ ID NO: 16)

HCDR3: TRQRGNYVGAMDY (SEQ ID NO: 17)

LCDR1: QDVSTA (SEQ ID NO: 18)

LCDR2: SAS (SEQ ID NO: 19)

LCDR3: HQYYGSPPT (SEQ ID NO: 20)

13E5 H1 heavy chain variable region

CAGGTGCAGCTGCAGCAGTCCGGAGCAGAGGTGGTGAAGCCAGGAGCCAG
CGTGAAGATCTCCTGTAAGACCTCTGGCTACACCTTCACAGAGTATACAA
TCCACTGGGTGAAGCAGGCACACGGACAGAGCCTGGAGTGGATCGGCGGC
ATCAACCCTAACAATGGCGGCACCGTGTACAATCAGAAGTTTCAGGGCAA
GGCCACCCTGACAGTGGACAAGTCTACCAGCACAGCCTATATGGAGCTGA
GGTCCCTGACCTCTGAGGACACAGCCGTGTACTATTGCGCCCGGGTGCGG
AGAGGCATGGATTACTGGGGCCAGGGCACCTCCGTGACAGTGAGCTCC (
SEQ ID NO: 21)

QVQLQQSGAEVVKPGASVKISCKTSGYTFTEYTIHWVKQAHGQSLEWIGG INPNNGGTVYNQKFQGKATLTVDKSTSTAYMELRSLTSEDTAVYYCARVR RGMDYWGQGTSVTVSS (SEQ ID NO: 22)

13E5 L1 light chain variable region

GACATCCAGATGACCCAGTCTCCTAAGTCTCTGAGCACATCCGTGGGCGA
CCGGGTGACCATCACATGTAGAGCCAGCCAGGATGTGACCACAGCAGTGG
CATGGTACCAGCAGAAGCCTGGCAAGTCCCCTAAGCTGCTGATCTATTCT
GCCAGCTACAGGTATACCGGAGTGCCATCTCGGTTCTCCGGCTCTGGCAG
CGGCACAGACTTCACCTTTACAATCAGCTCCGTGCAGCCAGAGGATCTGG
CCACCTACTATTGCCAGCAGCACTACAGCGCCCCATGGACCTTTGGCGGA
GGAACAAACCTGGAGATCAAG (SEQ ID NO: 23)

DIQMTQSPKSTSTSVGDRVTITCRASQDVTTAVAWYQQKPGKSPKLLIYS ASYRYTGVPSRFSGSGSGTDFTFTISSVQPEDLATYYCQQHYSAPWTFGG
GTNLEIK (SEQ ID NO: 24)

13E5 H2 heavy chain variable region

CAGGTGCAGCTGGTGCAGTCCGGAGCAGAGGTGGTGAAGCCAGGAGCCAG
CGTGAAGGTGTCCTGTAAGACCTCTGGCTACACCTTCACAGAGTATACAA
TCCACTGGGTGCGGCAGGCACCAGGACAGTCTCTGGAGTGGATCGGCGGC
ATCAACCCTAACAATGGCGGCACCAACTACAATCAGAAGTTTCAGGGCAA
GGTGACCCTGACAGTGGACAAGTCCACCTCTACAGCCTATATGGAGCTGA
GCTCCCTGAGGTCTGAGGACACAGCCGTGTACTATTGCGCCCGCGTGCGG
AGAGGCATGGATTACTGGGGCCAGGGCACCAGCGTGACAGTGTCTAGC (
SEQ ID NO: 25)

QVQLVQSGAEVVKPGASVKVSCKTSGYTFTEYTIHWVRQAPGQSLEWIGG INPNNGGTNYNQKFQGKVTLYVDKSTSTAYMELSSLRSEDTAVYYCARVR RGMDYWGQGTSVTVSS (SEQ ID NO: 26)

13E5 L2 light chain variable region

GACATCCAGATGACCCAGAGCCCTAGCTCCCTGAGCGCCTCCGTGGGCGA
CAGGGTGACCATCACATGTAGAGCCTCCCAGGATGTGACCACAGCAGTGG
CATGGTACCAGCAGAAGCCAGGCAAGGCCCCTAAGCTGCTGATCTACTCC
GCCTCTAGCAGGTATACCGGAGTGCCATCTCGGTTCTCTGGCAGCGGCTC
CGGCACAGACTTTACCCTGACAATCTCCTCTGTGCAGCCAGAGGATCTGG
CCACATACTATTGCCAGCAGCACTATTCTGCCCCCTGGACCTTTGGCGGC
GGCACAAACCTGGAGATCAAG (SEQ ID NO: 27)

DIQMTQSPSSLSASVGDRVTITCRASQDVTTAVAWYQQKPGKAPKLLIYS ASSRYTGVPSRFSGSGSGTDFTLTISSVQPEDLATYYCQQHYSAPWTFGG
GTNLEIK (SEQ ID NO: 28)

13E5 H3 heavy chain variable region

CAGGTGCAGCTGGTGCAGTCCGGAGCAGAGGTGGTGAAGCCAGGAGCCAG
CGTGAAGGTGTCCTGTAAGGCCTCTGGCTACACCTTCACAGAGTATACCA
TCCACTGGGTGCGGCAGGCACCAGGACAGGGACTGGAGTGGATCGGCGGC
ATCAACCCTAACAATGGCGGCACAAATTACGCCCAGAAGTTTCAGGGCAG
GGTGACCATCACAGTGGACAAGTCCACCTCTACAGCCTATATGGAGCTGA
GCTCCCTGAGGTCTGAGGACACCGCCGTGTACTATTGCGCCCGCGTGCGG
AGAGGCATGGATTACTGGGGCCAGGGCACCAGCGTGACAGTGTCTAGC (
SEQ ID NO: 29)

QVQLVQSGAEVVKPGASVKVSCKASGYTFTEYTIHWVRQAPGQGLEWIGG INPNNGGTNYAQKFQGRVTITVDKSTSTAYMELSSLRSEDTAVYYCARVR RGMDYWGQGTSVTVSS (SEQ ID NO: 30)

13E5 L3 light chain variable region

GACATCCAGATGACCCAGAGCCCTAGCTCCCTGAGCGCCTCCGTGGGCGA
CAGGGTGACCATCACATGTAGAGCCTCCCAGGATGTGACCACAGCCCTGG
CATGGTACCAGCAGAAGCCAGGCAAGGCCCCTAAGCTGCTGATCTACTCC
GCCTCTAGCCTGTATACCGGAGTGCCATCTCGGTTCTCTGGCAGCGGCTC
CGGCACAGACTTTACCCTGACAATCTCCTCTCTGCAGCCAGAGGATTTCG
CCACATACTATTGCCAGCAGCACTATTCTGCCCCCTGGACCTTTGGCGGC
GGCACAAACCTGGAGATCAAG (SEQ ID NO: 31)

DIQMTQSPSSLSASVGDRVTITCRASQDVTTALAWYQQKPGKAPKLLIYS ASSLYTGVPSRFSGSGSGTDFTLTISSLQPEDFATYYCOOHYSAPWTFGG
GTNLEIK (SEQ ID NO: 32)

13E5 H4 heavy chain variable region

CAGGTGCAGCTGGTGCAGTCCGGAGCAGAGGTGGTGAAGCCAGGAGCCAG
CGTGAAGGTGTCCTGTAAGACCTCTGGCTACACCTTCACAGAGTATACAA
TCCACTGGGTGCGGCAGGCACCAGGACAGTCTCTGGAGTGGATCGGCGGC
ATCAACCCTAACAATGGCGGCACCgtcTACAATCAGAAGTTTCAGGGCAA
GGTGACCCTGACAGTGGACAAGTCCACCTCTACAGCCTATATGGAGCTGA
GCTCCCTGAGGTCTGAGGACACAGCCGTGTACTATTGCGCCCGCGTGCGG
AGAGGCATGGATTACTGGGGCCAGGGCACCAGCGTGACAGTGTCTAGC (
SEQ ID NO: 33)

QVQLVQSGAEVVKPGASVKVSCKTSGYTFTEYTIHWVRQAPGQSLEWIGG INPNNGGTVYNQKFQGKVTLTVDKSTSTAYMELSSLRSEDTAVYYCARVR RGMDYWGQGTSVTVSS (SEQ ID NO: 34)

13E5 L4 light chain variable region

GACATCCAGATGACCCAGAGCCCTAGCTCCCTGAGCGCCTCCGTGGGCGA
CAGGGTGACCATCACATGTAGAGCCTCCCAGGATGTGACCACAGCAGTGG
CATGGTACCAGCAGAAGCCAGGCAAGGCCCCTAAGCTGCTGATCTACTCC
GCCTCTTAcAGGTATACCGGAGTGCCATCTCGGTTCTCTGGCAGCGGCTC
CGGCACAGACTTTACCCTGACAATCTCCTCTGTGCAGCCAGAGGATCTGG
CCACATACTATTGCCAGCAGCACTATTCTGCCCCCTGGACCTTTGGCGGC
GGCACAAACCTGGAGATCAAG (SEQ ID NO: 35)

DIQMTQSPSSLSASVGDRVTITCRASQDVTTAVAWYQQKPGKAPKLLIYS ASYRYTGVPSRFSGSGSGTDFTLTISSVQPEDLATYYCQQHYSAPWTFGG
GTNLEIK (SEQ ID NO: 36)

18H7 H1 heavy chain variable region

GACGTGCAGCTGGTGGAGAGCGGCGGCGGCCTGGTGCAGCTGGGCGGCTC
CCTGAGGCTGTCTTGTGCAGCCAGCGGCTTCACCTTTAGCTCCTCTTACA
TGTCTTGGGTGCGGCAGACACCCGAGAAGAGACTGGAGCTGGTGGCCGCC
ATCAACTCTAATGGCGGCAAGACCTACTATGCCGACAGCGTGAAGGGCAG
GTTTACCATCTCCCGCGATAACTCTAAGAATACACTGTATCTGCAGATGA
GCTCCCTGAGAGCCGAGGACACCGCCGTGTACTATTGCACACGGCAGAGA
GGCAACTACGTGGGCGCCATGGATTATTGGGGCCAGGGCACCAGCGTGAC
AGTGTCTAGC (SEQ ID NO: 37)

DVQLVESGGGLVQLGGSLRLSCAASGFTFSSSYMSWVRQTPEKRLELVAA INSNGGKTYYADSVKGRFTISRDNSKNTLYLQMSSLRAEDTAVYYCTROR GNYVGAMDYWGQGTSVTVSS (SEQ ID NO: 38)

18H7 L1 light chain variable region

GACATCCAGATGACCCAGTCTCCCAGCTCCCTGTCCACATCTGTGGGCGA
CCGGGTGACCATCACCTGTAAGGCCTCTCAGGATGTGAGCACCGCCGTGG
TGTGGTACCAGCAGAAGCCAGGCAAGAGCCCCAAGCTGCTGATCTATAGC
GCCTCCTACCGGTATACCGGCGTGCCTTCCAGATTCTCTGGCAGCGGCTC
CGGCACAGACTTCACCTTTACAATCTCTAGCGTGCAGCCAGAGGATATCG
CCACATACTATTGCCACCAGTACTATGGCAGCCCTCCCACCTTCGGCGGC
GGCACAAAGCTGGAGATCAAG (SEQ ID NO: 39)

DIQMTQSPSSLSTSVGDRVTITCKASQDVSTAVVWYQQKPGKSPKLLIYS ASYRYTGVPSRFSGSGSGTDFTFTISSVQPEDIATYYCHQYYGSPPTFGG
GTKLEIK (SEQ ID NO: 40)

18H7 H2 heavy chain variable region

GAGGTGCAGCTGGTGGAGAGCGGCGGCGGCCTGGTGCAGCCCGGCGGCTC
CCTGAGGCTGTCTTGTGCCGCCAGCGGCTTCACCTTTAGCTCCTCTTACA
TGTCTTGGGTGCGGCAGGCACCTGGCAAGAGACTGGAGCTGGTGGCCGCC
ATCAACTCTAATGGCGGCAAGACATACTATGCCGACAGCGTGAAGGGCAG
GTTTACCATCTCCCGCGATAACTCTAAGAATACACTGTATCTGCAGATGA
GCTCCCTGAGAGCCGAGGACACCGCCGTGTACTATTGCACACGGCAGAGA
GGCAACTACGTGGGCGCCATGGATTATTGGGGCCAGGGCACCAGCGTGAC
AGTGTCTAGC (SEQ ID NO: 41)

EVQLVESGGGLVQPGGSLRLSCAASGFTFSSSYMSWVRQAPGKRLELVAA INSNGGKTYYADSVKGRFTISRDNSKNTLYLQMSSLRAEDTAVYYCTRQR GNYVGAMDYWGQGTSVTVSS (SEQ ID NO: 42)

18H7 L2 light chain variable region

GACATCCAGATGACCCAGTCTCCAAGCTCCCTGTCCGCCTCTGTGGGCGA
CAGGGTGACCATCACATGTCAGGCCTCTCAGGATGTGAGCACAGCCGTGG
TGTGGTACCAGCAGAAGCCAGGCAAGAGCCCCAAGCTGCTGATCTATAGC
GCCTCCTACCTGTATACCGGCGTGCCTTCCAGATTCTCTGGCAGCGGCTC
CGGCACAGACTTCACCTTTACAATCTCTAGCCTGCAGCCAGAGGATATCG
CCACCTACTATTGCCACCAGTACTATGGCAGCCCTCCCACCTTCGGCGGC
GGCACAAAGCTGGAGATCAAG (SEQ ID NO: 43)

DIQMTQSPSSLSASVGDRVTITCQASQDVSTAVVWYQQKPGKSPKLLIYS ASYLYTGVPSRFSGSGSGTDFTFTISSLQPEDIATYYCHOYYGSPPTFGG
GTKLEIK (SEQ ID NO: 44)

18H7 H3 heavy chain variable region

GAGGTGCAGCTGGTGGAGAGCGGCGGCGGCCTGGTGCAGCCCGGCGGCTC
CCTGCGGCTGTCTTGTGCCGCCAGCGGCTTCACCTTTAGCTCCTCTTACA
TGTCCTGGGTGAGGCAGGCACCTGGCAAGGGACTGGAGTACGTGAGCGCC
ATCAACTCTAATGGCGGCAAGACATACTATGCCGACTCTGTGAAGGGCAG
GTTTACCATCAGCCGCGATAACTCCAAGAATACACTGTACCTGCAGATGA
GCTCCCTGAGGGCAGAGGACACCGCCGTGTACTATTGCACACGGCAGAGA
GGCAACTACGTGGGCGCCATGGATTATTGGGGCCAGGGCACCTCCGTGAC
AGTGTCTAGC (SEQ ID NO: 45)

EVQLVESGGGLVQPGGSLRLSCAASGFTFSSSYMSWVRQAPGKGLEYVSA INSNGGKTYYADSVKGRFTISRDNSKNTLYLQMSSLRAEDTAVYYCTRQR GNYVGAMDYWGQGTSVTVSS (SEQ ID NO: 46)

18H7 L3 light chain variable region

GACATCCAGATGACCCAGTCTCCAAGCTCCCTGTCCGCCTCTGTGGGCGA
CAGGGTGACCATCACATGTCAGGCCTCTCAGGATGTGAGCACAGCCCTGG
CCTGGTACCAGCAGAAGCCAGGCAAGGCCCCCAAGCTGCTGATCTATAGC
GCCTCCAACCTGGAGACCGGCGTGCCTAGCAGATTCTCTGGCAGCGGCTC
CGGCACAGACTTCACCTTTACAATCTCTAGCCTGCAGCCAGAGGATATCG
CCACCTACTATTGCCACCAGTACTATGGCAGCCCTCCCACCTTCGGCGGC
GGCACAAAGCTGGAGATCAAG (SEQ ID NO: 47)

DIQMTQSPSSLSASVGDRVTITCQASQDVSTALAWYQQKPGKAPKLLIYS ASNLETGVPSRFSGSGSGTDFTFTISSLQPEDIATYYCHOYYGSPPTFGG
GTKLEIK (SEQ ID NO: 48)

13E5 heavy chain framework region

FR-H1: EVQLQQSGPELVKPGASVKISCKTS (SEQ ID NO: 49)

FR-H2: IHWVKQNHGKSLEWIGG (SEQ ID NO: 50)

FR-H3: VYNQNFKGKATLTVDKSSSTAYMELRSLTSEDSAVYYC (SEQ
ID NO: 51)

FR-H4: WGQGTSVTVSS (SEQ ID NO: 52)

13E5 light chain framework region

FR-L1: DIVMTQSHKFMSTSVGDRVSITCKAS (SEQ ID NO: 53)

FR-L2: VAWYQQKPGQSPKLLIY (SEQ ID NO: 54)

FR-L3: YRYTGVPDRFTGSGSGTDFTFTISSVQAEDLAVYYC (SEQ I
D NO: 55)

FR-L4: FGGGTNLEIK (SEQ ID NO: 56)

18H7 heavy chain framework region

FR-H1: DVKLVESGGDLVNLGGSLKLSCAAS (SEQ ID NO: 57)

FR-H2: MSWVRQTPERRLELVAA (SEQ ID NO: 58)

FR-H3: YYPDTVKGRFTISRDNAKNTLYLQMSGLKTEDTALYYC (SEQ
ID NO: 59)

FR-H4: WGQGTSVTVSS (SEQ ID NO: 60)

18H7 light chain framework region

FR-L1: DIVMTQSHKFMSTSVGDRVSITCKAS (SEQ ID NO: 61)

FR-L2: VVWYQQKPGQSPTLLIY (SEQ ID NO:62)

FR-L3: YRYTGVPDRFTGSGSGTDFTFTISSVQAEDLAVYYC (SEQ I
D NO: 63)

FR-L4: FGGGTKLEIK (SEQ ID NO: 64)

13E5 H1 framework region

FR-H1: QVQLQQSGAEVVKPGASVKISCKTS (SEQ ID NO: 65)

FR-H2: IHWVKQAHGQSLEWIGG (SEQ ID NO: 66)

FR-H3: VYNQKFQGKATLTVDKSTSTAYMELRSLTSEDTAVYYC (SEQ
ID NO: 67)

FR-H4: WGQGTSVTVSS (SEQ ID NO: 68)

13E5 L1 framework region

FR-L1: DIQMTQSPKSLSTSVGDRVTITCRAS (SEQ ID NO: 69)

FR-L2: VAWYQQKPGKSPKLLIY (SEQ ID NO: 70)

FR-L3: YRYTGVPSRFSGSGSGTDFTFTISSVQPEDLATYYC (SEQ I
D NO: 71)

FR-L4: FGGGTNLEIK (SEQ ID NO: 72)

13E5 H2 framework region

FR-H1: QVQLVQSGAEVVKPGASVKVSCKTS (SEQ ID NO: 73)

FR-H2: IHWVRQAPGQSLEWIGG (SEQ ID NO: 74)

FR-H3: NYNQKFQGKVTLTVDKSTSTAYMELSSLRSEDTAVYYC (SEQ
ID NO: 75)

FR-H4: WGQGTSVTVSS (SEQ ID NO: 76)

13E5 L2 framework region

FR-L1: DIQMTQSPSSLSASVGDRVTITCRAS (SEQ ID NO: 77)

FR-L2: VAWYQQKPGKAPKLLIY (SEQ ID NO: 78)

FR-L3: SRYTGVPSRFSGSGSGTDFTLTISSVQPEDLATYYC (SEQ I
D NO: 79)

FR-L4: FGGGTNLEIK (SEQ ID NO: 80)

13E5 H3 framework region

FR-H1: QVQLVQSGAEVVKPGASVKVSCKAS (SEQ ID NO: 81)

FR-H2: IHWVRQAPGQGLEWIGG (SEQ ID NO: 82)

FR-H3: NYAQKFQGRVTITVDKSTSTAYMELSSLRSEDTAVYYC (SEQ
ID NO: 83)

FR-H4: WGQGTSVTVSS (SEQ ID NO: 84)

13E5 L3 framework region

FR-L1: DIQMTQSPSSLSASVGDRVTITCRAS (SEQ ID NO: 85)

FR-L2: LAWYQQKPGKAPKLLIY (SEQ ID NO: 86)

FR-L3: SLYTGVPSRFSGSGSGTDFTLTISSLQPEDFATYYC (SEQ I
D NO: 87)

FR-L4: FGGGTNLEIK (SEQ ID NO: 88)

13E5 H4 framework region

FR-H1: QVQLVQSGAEVVKPGASVKVSCKTS (SEQ ID NO: 89)

FR-H2: IHWVRQAPGQSLEWIGG (SEQ ID NO: 90)

FR-H3: VYNQKFQGKVTLTVDKSTSTAYMELSSLRSEDTAVYYC (SEQ
ID NO: 91)

FR-H4: WGQGTSVTVSS (SEQ ID NO: 92)

13E5 L4 framework region

FR-L1: DIQMTQSPSSLSASVGDRVTITCRAS (SEQ ID NO: 93)

FR-L2: VAWYQQKPGKAPKLLIY (SEQ ID NO: 94)

FR-L3: YRYTGVPSRFSGSGSGTDFTLTISSVQPEDLATYYC (SEQ I
D NO: 95)

FR-L4: FGGGTNLEIK (SEQ ID NO: 96)

18H7 H1 framework region

FR-H1: DVQLVESGGGLVQLGGSLRLSCAAS (SEQ ID NO: 97)

FR-H2: MSWVRQTPEKRLELVAA (SEQ ID NO: 98)

FR-H3: YYADSVKGRFTISRDNSKNTLYLQMSSLRAEDTAVYYC (SEQ
ID NO: 99)

FR-H4: WGQGTSVTVSS (SEQ ID NO: 100)

18H7 L1 framework region

FR-L1: DIQMTQSPSSLSTSVGDRVTITCKAS (SEQ ID NO: 101)

FR-L2: VVWYQQKPGKSPKLLIY (SEQ ID NO: 102)

FR-L3: YRYTGVPSRFSGSGSGTDFTFTISSVQPEDIATYYC (SEQ I
D NO: 103)

FR-L4: FGGGTKLEIK (SEQ ID NO: 104)

18H7 H2 framework region

FR-H1: EVQLVESGGGLVQPGGSLRLSCAAS (SEQ ID NO: 105)

FR-H2: MSWVRQAPGKRLELVAA (SEQ ID NO: 106)

FR-H3: YYADSVKGRFTISRDNSKNTLYLQMSSLRAEDTAVYYC (SEQ
ID NO: 107)

FR-H4: WGQGTSVTVSS (SEQ ID NO: 108)

18H7 L2 framework region

FR-L1: DIQMTQSPSSLSASVGDRVTITCQAS (SEQ ID NO: 109)

FR-L2: VVWYQQKPGKSPKLLIY (SEQ ID NO: 110)

FR-L3: YLYTGVPSRFSGSGSGTDFTFTISSLQPEDIATYYC (SEQ I
D NO: 111)

FR-L4: FGGGTKLEIK (SEQ ID NO: 112)

18H7 H3 framework region

FR-H1: EVQLVESGGGLVQPGGSLRLSCAAS (SEQ ID NO: 113)

FR-H2: MSWVRQAPGKGLEYVSA (SEQ ID NO: 114)

FR-H3: YYADSVKGRFTISRDNSKNTLYLQMSSLRAEDTAVYYC (SEQ
ID NO: 115)

FR-H4: WGQGTSVTVSS (SEQ ID NO: 116)

18H7 L3 framework region

FR-L1: DIQMTQSPSSLSASVGDRVTITCQAS (SEQ ID NO: 117)

FR-L2: LAWYQQKPGKAPKLLIY (SEQ ID NO: 118)

FR-L3: NLETGVPSRFSGSGSGTDFTFTISSLQPEDIATYYC (SEQ I
D NO: 119)

FR-L4: FGGGTKLEIK (SEQ ID NO: 120)

Sequence of mFc tag:

PRGPTIKPCPPCKCPAPNLLGGPSVFIFPPKIKDVLMISLSPIVTCVVVD
VSEDDPDVQISWFVNNVEVHTAQTQTHREDYNSTLRVVSALPIQHQDWMS
GKEFKCKVNNKDLPAPIERTISKPKGSVRAPQVYVLPPPEEEMTKKQVTL
TCMVTDFMPEDIYVEWTNNGKTELNYKNTEPVLDSDGSYFMYSKLRVEKK
NWVERNSYSCSVVHEGLHNHHTTKSFSRTPGK (SEQ ID NO: 121)

hIgG heavy chain sequence

EVQLEQSGAELMKPGASVKISCKATGYTFTTYWIEWIKQRPGHSLEWIGE
ILPGSDSTYYNEKVKGKVTFTADASSNTAYMQLSSLTSEDSAVYYCARGD
GFYVYWGQGTTLTVSSASTKGPSVFPLAPCSRSTSESTAALGCLVKDYFP
EPVTVSWNSGALTSGVHTFPAVLQSSGLYSLSSVVTVPSSSLGTKTYTCN
VDHKPSNTKVDKRVESKYGPPCPPCPAPEFLGGPSVFLFPPKPKDTLMIS
RTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK (SEQ I
D NO: 122)

hIgG light chain sequence

DIELTQSPATLSVTPGDSVSLSCRASQSISNNLHWYQQKSHESPRLLIKY
TSQSMSGIPSRFSGSGSGTDFTLSINSVETEDFGVYFCQQSGSWPRTFGG
GTKLDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC (SEQ ID NO: 123)

VAB 16F3-1 VH-2

EVQLVESGGGLVQPGRSLRLSCEASGFTFDDYAMHWVRQAPGKGLEWVSG LSRTSVSIGYADSVKGRFTISRDNAKNSLYLEMNSLRPEDTALYYCAKWG TRGYFDYWGQGTLVTVSS (SEQ ID NO: 124)

16F3-1 VL

DIQMTQSPSSVSASVGDRVTITCRASQDISIWLAWYQQSPGKAPKLLINV ASRLQSGVPSRFSGSGSGTDFTLTINSLQPEDFVTYYCQQANSFPITFGQ
GTRLATK (SEQ ID NO: 125)

20G 10H3 heavy chain variable region

CAGGTGACACTGAAGGAGTCCGGCCCAACCCTGGTGAAGCCCACCCAGAC
ACTGACCCTGACATGTACCTTCAGCGGCTTTTCTCTGAGCACATCCGGCA
TGGGAGTGGGATGGATCAGACAGCCCCCTGGCAAGGCCCTGGAGTGGCTG
GCCCTGATCTGGTGGGCCGACGATAAGAGGTACTCTACCAGCCTGAAGTC
CCGCCTGACAATCTCCAAGGACACCTCTAAGAACCAGGTGGTGCTGACAA
TCACCAATGTGGACCCCGTGGATACAGCCACCTACTATTGCGCCCGGATC
ACAAGAGGCAACTCTGCCATGGATTTTTGGGGCCAGGGCACATCTGTGAC
CGTGAGCTCC (SEQ ID NO: 126)

QVTLKESGPTLVKPTQTLTLTCTFSGFSLSTSGMGVGWIRQPPGKALEWL
ALIWWADDKRYSTSLKSRLTISKDTSKNQVVLTITNVDPVDTATYYCARI
TRGNSAMDFWGQGTSVTVSS (SEQ ID NO: 127)

20G 10L3 light chain variable region

GACATCCAGATGACCCAGTCTCCTAGCTCCCTGTCCGCCTCTGTGGGCGA
TAGGGTGACCATCACATGTAGAGCCTCTGAGAACGTGTACAGCTATCTGA
ATTGGTACCAGCAGAAGCCCGGCAAGGCCCCTAAGCTGCTGATCTATAAC
GCCAAGAGCCTGCAGTCCGGAGTGCCAAGCCGGTTCAGCGGCTCCGGCTC
TGGCACCGACTTTACCCTGACAATCTCTAGCCTGCAGCCAGAGGATTTCG
CCACATACTATTGCCAGCACCACTACGGCATCCCCTGGACCTTCGGCGGC
GGCACAAAGCTGGAGATCAAG (SEQ ID NO:128)

DIQMTQSPSSLSASVGDRVTITCRASENVYSYLNWYQQKPGKAPKLLIYN
AKSLQSGVPSRFSGSGSGTDFTLTISSLQPEDFATYYCQHHYGIPWTFGG
GTKLEIK (SEQ ID NO: 129)

20G10 CDR

HCDR1: GFSLSTSGMG (SEQ ID NO: 130)

HCDR2: IWWADDK (SEQ ID NO: 131)

HCDR3: ARITRGNSAMDF (SEQ ID NO: 132)

LCDR1: ENVYSY (SEQ ID NO: 133)

LCDR2: NAK (SEQ ID NO: 134)

LCDR3: QHHYGIPWT (SEQ ID NO: 135)

20G 10H3 framework region

FR-H1: QVTLKESGPTLVKPTQTLTLTCTFS (SEQ ID NO: 136)

FR-H2: VGWIRQPPGKALEWLAL (SEQ ID NO: 137)

FR-H3: RYSTSLKSRLTISKDTSKNQVVLTITNVDPVDTATYYC (SEQ
ID NO: 138)

FR-H4: WGQGTSVTVSS (SEQ ID NO: 139)

20G 10L3 framework region

FR-L1: DIQMTQSPSSLSASVGDRVTITCRAS (SEQ ID NO: 140)

FR-L2: LNWYQQKPGKAPKLLIY (SEQ ID NO: 141)

FR-L3: SLQSGVPSRFSGSGSGTDFTLTISSLQPEDFATYYC (SEQ I
D NO: 142)

FR-L4: FGGGTKLEIK (SEQ ID NO: 143)

1. An antibody or an antigen-binding fragment thereof binding to IL4RA, preferably human IL4RA, wherein: (1) the antibody comprises:
an HCDR1, comprising or consisting of the sequence set forth in SEQ ID NO: 9, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence,
an HCDR2, comprising or consisting of the sequence set forth in SEQ ID NO: 10, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence, and
an HCDR3, comprising or consisting of the sequence set forth in SEQ ID NO: 11, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence,
and the antibody further comprises:
an LCDR1, comprising or consisting of the sequence set forth in SEQ ID NO: 12, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence,
an LCDR2, comprising or consisting of the sequence set forth in SEQ ID NO: 13, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence, and
an LCDR3, comprising or consisting of the sequence set forth in SEQ ID NO: 14, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence;
(2) the antibody comprises:
an HCDR1, comprising or consisting of the sequence set forth in SEQ ID NO: 15, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence,
an HCDR2, comprising or consisting of the sequence set forth in SEQ ID NO: 16, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence, and
an HCDR3, comprising or consisting of the sequence set forth in SEQ ID NO: 17, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence,
and the antibody further comprises:
an LCDR1, comprising or consisting of the sequence set forth in SEQ ID NO: 18, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence,
an LCDR2, comprising or consisting of the sequence set forth in SEQ ID NO: 19, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence, and
an LCDR3, comprising or consisting of the sequence set forth in SEQ ID NO: 20, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence;
or, (3) the antibody comprises:
an HCDR1, comprising or consisting of the sequence set forth in SEQ ID NO: 130, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence,
an HCDR2, comprising or consisting of the sequence set forth in SEQ ID NO: 131, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence, and
an HCDR3, comprising or consisting of the sequence set forth in SEQ ID NO: 132, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence,
and the antibody further comprises:
an LCDR1, comprising or consisting of the sequence set forth in SEQ ID NO: 133, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence,
an LCDR2, comprising or consisting of the sequence set forth in SEQ ID NO: 134, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence, and
an LCDR3, comprising or consisting of the sequence set forth in SEQ ID NO: 135, a sequence having at least 90%, preferably at least 91%, 92%, 93%, 94%, 95%, 96%, 97%, 98% or 99% sequence identity to the sequence, or an amino acid sequence having one or more (preferably 2 or 3) conservative amino acid mutations (preferably substitutions, insertions or deletions) compared to the sequence,
or the antibody is secreted by a hybridoma cell, wherein the hybridoma cell has the accession number of CCTCC NO: C202010, CCTCC NO: C2018131 or CCTCC NO: C2018132.

2. The antibody according to claim 1, comprising:
(A) (i) a heavy chain variable region comprising or consisting of:
the amino acid sequence set forth in SEQ ID NO: 2, a sequence having at least 80%, 85% or 90%, preferably at least 91%, 92%, 93%, 94%, 95%, 96%, 97%, 98% or 99% sequence identity to the sequence set forth in SEQ ID NO: 2, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 2, and
(ii) a light chain variable region comprising or consisting of:
the amino acid sequence set forth in SEQ ID NO: 4, a sequence having at least 80%, 85% or 90%, preferably at least 91%, 92%, 93%, 94%, 95%, 96%, 97%, 98% or 99% sequence identity to the sequence set forth in SEQ ID NO: 4, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 4;
(B) (i) a heavy chain variable region comprising or consisting of: the amino acid sequence set forth in SEQ ID NO: 6, a sequence having at least 80%, 85% or 90%, preferably at least 91%, 92%, 93%, 94%, 95%, 96%, 97%, 98% or 99% sequence identity to the sequence set forth in SEQ ID NO: 6, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 6, and
(ii) a light chain variable region comprising or consisting of: the amino acid sequence set forth in SEQ ID NO: 8, a sequence having at least 80%, 85% or 90%, preferably at least 91%, 92%, 93%, 94%, 95%, 96%, 97%, 98% or 99% sequence identity to the sequence set forth in SEQ ID NO: 8, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 8;
(C) (i) a heavy chain variable region comprising or consisting of:
the amino acid sequence set forth in SEQ ID NO: 22, a sequence having at least 80%, 85% or 90%, preferably at least 91%, 92%, 93%, 94%, 95%, 96%, 97%, 98% or 99% sequence identity to the sequence set forth in SEQ ID NO: 22, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 22, and
(ii) a light chain variable region comprising or consisting of: the amino acid sequence set forth in SEQ ID NO: 24, a sequence having at least 80%, 85% or 90%, preferably at least 91%, 92%, 93%, 94%, 95%, 96%, 97%, 98% or 99% sequence identity to the sequence set forth in SEQ ID NO: 24, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 24;
(D) (i) a heavy chain variable region comprising or consisting of:
the amino acid sequence set forth in SEQ ID NO: 26, a sequence having at least 80%, 85% or 90%, preferably at least 91%, 92%, 93%, 94%, 95%, 96%, 97%, 98% or 99% sequence identity to the sequence set forth in SEQ ID NO: 26, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 26, and
(ii) a light chain variable region comprising or consisting of:
the amino acid sequence set forth in SEQ ID NO: 28, a sequence having at least 80%, 85% or 90%, preferably at least 91%, 92%, 93%, 94%, 95%, 96%, 97%, 98% or 99% sequence identity to the sequence set forth in SEQ ID NO: 28, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 28;
(E) (i) a heavy chain variable region comprising or consisting of:
the amino acid sequence set forth in SEQ ID NO: 30, a sequence having at least 80%, 85% or 90%, preferably at least 91%, 92%, 93%, 94%, 95%, 96%, 97%, 98% or 99% sequence identity to the sequence set forth in SEQ ID NO: 30, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 30, and
(ii) a light chain variable region comprising or consisting of:
the amino acid sequence set forth in SEQ ID NO: 32, a sequence having at least 80%, 85% or 90%, preferably at least 91%, 92%, 93%, 94%, 95%, 96%, 97%, 98% or 99% sequence identity to the sequence set forth in SEQ ID NO: 32, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 32;
(F) (i) a heavy chain variable region comprising or consisting of:
the amino acid sequence set forth in SEQ ID NO: 34, a sequence having at least 80%, 85% or 90%, preferably at least 91%, 92%, 93%, 94%, 95%, 96%, 97%, 98% or 99% sequence identity to the sequence set forth in SEQ ID NO: 34, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 34, and
(ii) a light chain variable region comprising or consisting of:
the amino acid sequence set forth in SEQ ID NO: 36, a sequence having at least 80%, 85% or 90%, preferably at least 91%, 92%, 93%, 94%, 95%, 96%, 97%, 98% or 99% sequence identity to the sequence set forth in SEQ ID NO: 36, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 36;
(G) (i) a heavy chain variable region comprising or consisting of:
the amino acid sequence set forth in SEQ ID NO: 38, a sequence having at least 80%, 85% or 90%, preferably at least 91%, 92%, 93%, 94%, 95%, 96%, 97%, 98% or 99% sequence identity to the sequence set forth in SEQ ID NO: 38, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 38, and
(ii) a light chain variable region comprising or consisting of:
the amino acid sequence set forth in SEQ ID NO: 40, a sequence having at least 80%, 85% or 90%, preferably at least 91%, 92%, 93%, 94%, 95%, 96%, 97%, 98% or 99% sequence identity to the sequence set forth in SEQ ID NO: 40, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 40;
(H) (i) a heavy chain variable region comprising or consisting of:
the amino acid sequence set forth in SEQ ID NO: 42, a sequence having at least 80%, 85% or 90%, preferably at least 91%, 92%, 93%, 94%, 95%, 96%, 97%, 98% or 99% sequence identity to the sequence set forth in SEQ ID NO: 42, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 42, and
(ii) a light chain variable region comprising or consisting of:
the amino acid sequence set forth in SEQ ID NO: 44, a sequence having at least 80%, 85% or 90%, preferably at least 91%, 92%, 93%, 94%, 95%, 96%, 97%, 98% or 99% sequence identity to the sequence set forth in SEQ ID NO: 44, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 44;
(I) (i) a heavy chain variable region comprising or consisting of:
the amino acid sequence set forth in SEQ ID NO: 46, a sequence having at least 80%, 85% or 90%, preferably at least 91%, 92%, 93%, 94%, 95%, 96%, 97%, 98% or 99% sequence identity to the sequence set forth in SEQ ID NO: 46, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 46, and
(ii) a light chain variable region comprising or consisting of:
the amino acid sequence set forth in SEQ ID NO: 48, a sequence having at least 80%, 85% or 90%, preferably at least 91%, 92%, 93%, 94%, 95%, 96%, 97%, 98% or 99% sequence identity to the sequence set forth in SEQ ID NO: 48, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 48;
(J) (i) a heavy chain variable region comprising or consisting of:
the amino acid sequence set forth in SEQ ID NO: 42, a sequence having at least 80%, 85% or 90%, preferably at least 91%, 92%, 93%, 94%, 95%, 96%, 97%, 98% or 99% sequence identity to the sequence set forth in SEQ ID NO: 42, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 42, and
(ii) a light chain variable region comprising or consisting of:
the amino acid sequence set forth in SEQ ID NO: 48, a sequence having at least 80%, 85% or 90%, preferably at least 91%, 92%, 93%, 94%, 95%, 96%, 97%, 98% or 99% sequence identity to the sequence set forth in SEQ ID NO: 48, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 48;
(K) (i) a heavy chain variable region comprising or consisting of:
the amino acid sequence set forth in SEQ ID NO: 46, a sequence having at least 80%, 85% or 90%, preferably at least 91%, 92%, 93%, 94%, 95%, 96%, 97%, 98% or 99% sequence identity to the sequence set forth in SEQ ID NO: 46, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 46, and
(ii) a light chain variable region comprising or consisting of:
the amino acid sequence set forth in SEQ ID NO: 44, a sequence having at least 80%, 85% or 90%, preferably at least 91%, 92%, 93%, 94%, 95%, 96%, 97%, 98% or 99% sequence identity to the sequence set forth in SEQ ID NO: 44, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 44;
(L) (i) a heavy chain variable region comprising or consisting of:
the amino acid sequence set forth in SEQ ID NO: 34, a sequence having at least 80%, 85% or 90%, preferably at least 91%, 92%, 93%, 94%, 95%, 96%, 97%, 98% or 99% sequence identity to the sequence set forth in SEQ ID NO: 34, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 34, and
(ii) a light chain variable region comprising or consisting of:
the amino acid sequence set forth in SEQ ID NO: 28, a sequence having at least 80%, 85% or 90%, preferably at least 91%, 92%, 93%, 94%, 95%, 96%, 97%, 98% or 99% sequence identity to the sequence set forth in SEQ ID NO: 28, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 28; or
(M) (i) a heavy chain variable region comprising or consisting of:
the amino acid sequence set forth in SEQ ID NO: 127, a sequence having at least 80%, 85% or 90%, preferably at least 91%, 92%, 93%, 94%, 95%, 96%, 97%, 98% or 99% sequence identity to the sequence set forth in SEQ ID NO: 127, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 127, and
(ii) a light chain variable region comprising or consisting of:
the amino acid sequence set forth in SEQ ID NO: 129, a sequence having at least 80%, 85% or 90%, preferably at least 91%, 92%, 93%, 94%, 95%, 96%, 97%, 98% or 99% sequence identity to the sequence set forth in SEQ ID NO: 129, or an amino acid sequence having one or more (preferably 1, 2, 3, 4, 5, 6, 7, 8, 9 or 10) conservative amino acid mutations (preferably substitutions, insertions or deletions) compared to the sequence set forth in SEQ ID NO: 129.

3. The antibody or the antigen-binding fragment thereof according to claim 1, wherein the antibody further comprises framework regions FR-H1, FR-H2, FR-H3 and FR-H4 in the heavy chain variable region, and the framework regions FR-L1, FR-L2, FR-L3 and FR-L4 in the light chain variable region, wherein
(A) the FR-H1 comprises or consists of the amino acid sequence set forth in SEQ ID NO: 49, a sequence having at least 80%, 85% or 90%, preferably at least 91%, 92%, 93%, 94%, 95%, 96%, 97%, 98% or 99% sequence identity to the sequence set forth in SEQ ID NO: 49, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 49, the FR-H2 comprises or consists of the amino acid sequence set forth in SEQ ID NO: 50, a sequence having at least 80%, 85% or 90%, preferably at least 91%, 92%, 93%, 94%, 95%, 96%, 97%, 98% or 99% sequence identity to the sequence set forth in SEQ ID NO: 50, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 50, the FR-H3 comprises or consists of the amino acid sequence set forth in SEQ ID NO: 51, a sequence having at least 80%, 85% or 90%, preferably at least 91%, 92%, 93%, 94%, 95%, 96%, 97%, 98% or 99% sequence identity to the sequence set forth in SEQ ID NO: 51, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 51, the FR-H4 comprises or consists of the amino acid sequence set forth in SEQ ID NO: 52, a sequence having at least 80%, 85% or 90%, preferably at least 91%, 92%, 93%, 94%, 95%, 96%, 97%, 98% or 99% sequence identity to the sequence set forth in SEQ ID NO: 52, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 52, the FR-L1 comprises or consists of the amino acid sequence set forth in SEQ ID NO: 53, a sequence having at least 80%, 85% or 90%, preferably at least 91%, 92%, 93%, 94%, 95%, 96%, 97%, 98% or 99% sequence identity to the sequence set forth in SEQ ID NO: 53, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 53, the FR-L2 comprises or consists of the amino acid sequence set forth in SEQ ID NO: 54, a sequence having at least 80%, 85% or 90%, preferably at least 91%, 92%, 93%, 94%, 95%, 96%, 97%, 98% or 99% sequence identity to the sequence set forth in SEQ ID NO: 54, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 54, the FR-L3 comprises or consists of the amino acid sequence set forth in SEQ ID NO: 55, a sequence having at least 80%, 85% or 90%, preferably at least 91%, 92%, 93%, 94%, 95%, 96%, 97%, 98% or 99% sequence identity to the sequence set forth in SEQ ID NO: 55, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 55, and the FR-L4 comprises or consists of the amino acid sequence set forth in SEQ ID NO: 56, a sequence having at least 80%, 85% or 90%, preferably at least 91%, 92%, 93%, 94%, 95%, 96%, 97%, 98% or 99% sequence identity to the sequence set forth in SEQ ID NO: 56, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 56;
(B) the FR-H1 comprises or consists of the amino acid sequence set forth in SEQ ID NO: 57, a sequence having at least 80%, 85% or 90%, preferably at least 91%, 92%, 93%, 94%, 95%, 96%, 97%, 98% or 99% sequence identity to the sequence set forth in SEQ ID NO: 57, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 57,
the FR-H2 comprises or consists of the amino acid sequence set forth in SEQ ID NO: 58, a sequence having at least 80%, 85% or 90%, preferably at least 91%, 92%, 93%, 94%, 95%, 96%, 97%, 98% or 99% sequence identity to the sequence set forth in SEQ ID NO: 58, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 58,
the FR-H3 comprises or consists of the amino acid sequence set forth in SEQ ID NO: 59, a sequence having at least 80%, 85% or 90%, preferably at least 91%, 92%, 93%, 94%, 95%, 96%, 97%, 98% or 99% sequence identity to the sequence set forth in SEQ ID NO: 59, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 59,
the FR-H4 comprises or consists of the amino acid sequence set forth in SEQ ID NO: 60, a sequence having at least 80%, 85% or 90%, preferably at least 91%, 92%, 93%, 94%, 95%, 96%, 97%, 98% or 99% sequence identity to the sequence set forth in SEQ ID NO: 60, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 60,
the FR-L1 comprises or consists of the amino acid sequence set forth in SEQ ID NO: 61, a sequence having at least 80%, 85% or 90%, preferably at least 91%, 92%, 93%, 94%, 95%, 96%, 97%, 98% or 99% sequence identity to the sequence set forth in SEQ ID NO: 61, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 61,
the FR-L2 comprises or consists of the amino acid sequence set forth in SEQ ID NO: 62, a sequence having at least 80%, 85% or 90%, preferably at least 91%, 92%, 93%, 94%, 95%, 96%, 97%, 98% or 99% sequence identity to the sequence set forth in SEQ ID NO: 62, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 62,
the FR-L3 comprises or consists of the amino acid sequence set forth in SEQ ID NO: 63, a sequence having at least 80%, 85% or 90%, preferably at least 91%, 92%, 93%, 94%, 95%, 96%, 97%, 98% or 99% sequence identity to the sequence set forth in SEQ ID NO: 63, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 63, and
the FR-L4 comprises or consists of the amino acid sequence set forth in SEQ ID NO: 64, a sequence having at least 80%, 85% or 90%, preferably at least 91%, 92%, 93%, 94%, 95%, 96%, 97%, 98% or 99% sequence identity to the sequence set forth in SEQ ID NO: 64, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 64;
(C) the FR-H1 comprises or consists of the amino acid sequence set forth in SEQ ID NO: 65, a sequence having at least 80%, 85% or 90%, preferably at least 91%, 92%, 93%, 94%, 95%, 96%, 97%, 98% or 99% sequence identity to the sequence set forth in SEQ ID NO: 65, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 65,
the FR-H2 comprises or consists of the amino acid sequence set forth in SEQ ID NO: 66, a sequence having at least 80%, 85% or 90%, preferably at least 91%, 92%, 93%, 94%, 95%, 96%, 97%, 98% or 99% sequence identity to the sequence set forth in SEQ ID NO: 66, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 66,
the FR-H3 comprises or consists of the amino acid sequence set forth in SEQ ID NO: 67, a sequence having at least 80%, 85% or 90%, preferably at least 91%, 92%, 93%, 94%, 95%, 96%, 97%, 98% or 99% sequence identity to the sequence set forth in SEQ ID NO: 67, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 67,
the FR-H4 comprises or consists of the amino acid sequence set forth in SEQ ID NO: 68, a sequence having at least 80%, 85% or 90%, preferably at least 91%, 92%, 93%, 94%, 95%, 96%, 97%, 98% or 99% sequence identity to the sequence set forth in SEQ ID NO: 68, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 68,
the FR-L1 comprises or consists of the amino acid sequence set forth in SEQ ID NO: 69, a sequence having at least 80%, 85% or 90%, preferably at least 91%, 92%, 93%, 94%, 95%, 96%, 97%, 98% or 99% sequence identity to the sequence set forth in SEQ ID NO: 69, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 69,
the FR-L2 comprises or consists of the amino acid sequence set forth in SEQ ID NO: 70, a sequence having at least 80%, 85% or 90%, preferably at least 91%, 92%, 93%, 94%, 95%, 96%, 97%, 98% or 99% sequence identity to the sequence set forth in SEQ ID NO: 70, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 70,
the FR-L3 comprises or consists of the amino acid sequence set forth in SEQ ID NO: 71, a sequence having at least 80%, 85% or 90%, preferably at least 91%, 92%, 93%, 94%, 95%, 96%, 97%, 98% or 99% sequence identity to the sequence set forth in SEQ ID NO: 71, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 71, and
the FR-L4 comprises or consists of the amino acid sequence set forth in SEQ ID NO: 72, a sequence having at least 80%, 85% or 90%, preferably at least 91%, 92%, 93%, 94%, 95%, 96%, 97%, 98% or 99% sequence identity to the sequence set forth in SEQ ID NO: 72, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 72;
(D) the FR-H1 comprises or consists of the amino acid sequence set forth in SEQ ID NO: 73, a sequence having at least 80%, 85% or 90%, preferably at least 91%, 92%, 93%, 94%, 95%, 96%, 97%, 98% or 99% sequence identity to the sequence set forth in SEQ ID NO: 73, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 73,
the FR-H2 comprises or consists of the amino acid sequence set forth in SEQ ID NO: 74, a sequence having at least 80%, 85% or 90%, preferably at least 91%, 92%, 93%, 94%, 95%, 96%, 97%, 98% or 99% sequence identity to the sequence set forth in SEQ ID NO: 74, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 74,
the FR-H3 comprises or consists of the amino acid sequence set forth in SEQ ID NO: 75, a sequence having at least 80%, 85% or 90%, preferably at least 91%, 92%, 93%, 94%, 95%, 96%, 97%, 98% or 99% sequence identity to the sequence set forth in SEQ ID NO: 75, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 75,
the FR-H4 comprises or consists of the amino acid sequence set forth in SEQ ID NO: 76, a sequence having at least 80%, 85% or 90%, preferably at least 91%, 92%, 93%, 94%, 95%, 96%, 97%, 98% or 99% sequence identity to the sequence set forth in SEQ ID NO: 76, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 76,
the FR-L1 comprises or consists of the amino acid sequence set forth in SEQ ID NO: 77, a sequence having at least 80%, 85% or 90%, preferably at least 91%, 92%, 93%, 94%, 95%, 96%, 97%, 98% or 99% sequence identity to the sequence set forth in SEQ ID NO: 77, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 77,
the FR-L2 comprises or consists of the amino acid sequence set forth in SEQ ID NO: 78, a sequence having at least 80%, 85% or 90%, preferably at least 91%, 92%, 93%, 94%, 95%, 96%, 97%, 98% or 99% sequence identity to the sequence set forth in SEQ ID NO: 78, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 78,
the FR-L3 comprises or consists of the amino acid sequence set forth in SEQ ID NO: 79, a sequence having at least 80%, 85% or 90%, preferably at least 91%, 92%, 93%, 94%, 95%, 96%, 97%, 98% or 99% sequence identity to the sequence set forth in SEQ ID NO: 79, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 79, and
the FR-L4 comprises or consists of the amino acid sequence set forth in SEQ ID NO: 80, a sequence having at least 80%, 85% or 90%, preferably at least 91%, 92%, 93%, 94%, 95%, 96%, 97%, 98% or 99% sequence identity to the sequence set forth in SEQ ID NO: 80, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 80;
(E) the FR-H1 comprises or consists of the amino acid sequence set forth in SEQ ID NO: 81, a sequence having at least 80%, 85% or 90%, preferably at least 91%, 92%, 93%, 94%, 95%, 96%, 97%, 98% or 99% sequence identity to the sequence set forth in SEQ ID NO: 81, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 81,
the FR-H2 comprises or consists of the amino acid sequence set forth in SEQ ID NO: 82, a sequence having at least 80%, 85% or 90%, preferably at least 91%, 92%, 93%, 94%, 95%, 96%, 97%, 98% or 99% sequence identity to the sequence set forth in SEQ ID NO: 82, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 82,
the FR-H3 comprises or consists of the amino acid sequence set forth in SEQ ID NO: 83, a sequence having at least 80%, 85% or 90%, preferably at least 91%, 92%, 93%, 94%, 95%, 96%, 97%, 98% or 99% sequence identity to the sequence set forth in SEQ ID NO: 83, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 83,
the FR-H4 comprises or consists of the amino acid sequence set forth in SEQ ID NO: 84, a sequence having at least 80%, 85% or 90%, preferably at least 91%, 92%, 93%, 94%, 95%, 96%, 97%, 98% or 99% sequence identity to the sequence set forth in SEQ ID NO: 84, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 84,
the FR-L1 comprises or consists of the amino acid sequence set forth in SEQ ID NO: 85, a sequence having at least 80%, 85% or 90%, preferably at least 91%, 92%, 93%, 94%, 95%, 96%, 97%, 98% or 99% sequence identity to the sequence set forth in SEQ ID NO: 85, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 85,
the FR-L2 comprises or consists of the amino acid sequence set forth in SEQ ID NO: 86, a sequence having at least 80%, 85% or 90%, preferably at least 91%, 92%, 93%, 94%, 95%, 96%, 97%, 98% or 99% sequence identity to the sequence set forth in SEQ ID NO: 86, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 86,
the FR-L3 comprises or consists of the amino acid sequence set forth in SEQ ID NO: 87, a sequence having at least 80%, 85% or 90%, preferably at least 91%, 92%, 93%, 94%, 95%, 96%, 97%, 98% or 99% sequence identity to the sequence set forth in SEQ ID NO: 87, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 87, and
the FR-L4 comprises or consists of the amino acid sequence set forth in SEQ ID NO: 88, a sequence having at least 80%, 85% or 90%, preferably at least 91%, 92%, 93%, 94%, 95%, 96%, 97%, 98% or 99% sequence identity to the sequence set forth in SEQ ID NO: 88, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 88;
(F) the FR-H1 comprises or consists of the amino acid sequence set forth in SEQ ID NO: 89, a sequence having at least 80%, 85% or 90%, preferably at least 91%, 92%, 93%, 94%, 95%, 96%, 97%, 98% or 99% sequence identity to the sequence set forth in SEQ ID NO: 89, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 89,
the FR-H2 comprises or consists of the amino acid sequence set forth in SEQ ID NO: 90, a sequence having at least 80%, 85% or 90%, preferably at least 91%, 92%, 93%, 94%, 95%, 96%, 97%, 98% or 99% sequence identity to the sequence set forth in SEQ ID NO: 90, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 90,
the FR-H3 comprises or consists of the amino acid sequence set forth in SEQ ID NO: 91, a sequence having at least 80%, 85% or 90%, preferably at least 91%, 92%, 93%, 94%, 95%, 96%, 97%, 98% or 99% sequence identity to the sequence set forth in SEQ ID NO: 91, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 91,
the FR-H4 comprises or consists of the amino acid sequence set forth in SEQ ID NO: 92, a sequence having at least 80%, 85% or 90%, preferably at least 91%, 92%, 93%, 94%, 95%, 96%, 97%, 98% or 99% sequence identity to the sequence set forth in SEQ ID NO: 92, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 92,
the FR-L1 comprises or consists of the amino acid sequence set forth in SEQ ID NO: 93, a sequence having at least 80%, 85% or 90%, preferably at least 91%, 92%, 93%, 94%, 95%, 96%, 97%, 98% or 99% sequence identity to the sequence set forth in SEQ ID NO: 93, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 93,
the FR-L2 comprises or consists of the amino acid sequence set forth in SEQ ID NO: 94, a sequence having at least 80%, 85% or 90%, preferably at least 91%, 92%, 93%, 94%, 95%, 96%, 97%, 98% or 99% sequence identity to the sequence set forth in SEQ ID NO: 94, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 94,
the FR-L3 comprises or consists of the amino acid sequence set forth in SEQ ID NO: 95, a sequence having at least 80%, 85% or 90%, preferably at least 91%, 92%, 93%, 94%, 95%, 96%, 97%, 98% or 99% sequence identity to the sequence set forth in SEQ ID NO: 95, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 95, and
the FR-L4 comprises or consists of the amino acid sequence set forth in SEQ ID NO: 96, a sequence having at least 80%, 85% or 90%, preferably at least 91%, 92%, 93%, 94%, 95%, 96%, 97%, 98% or 99% sequence identity to the sequence set forth in SEQ ID NO: 96, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 96;
(G) the FR-H1 comprises or consists of the amino acid sequence set forth in SEQ ID NO: 97, a sequence having at least 80%, 85% or 90%, preferably at least 91%, 92%, 93%, 94%, 95%, 96%, 97%, 98% or 99% sequence identity to the sequence set forth in SEQ ID NO: 97, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 97,
the FR-H2 comprises or consists of the amino acid sequence set forth in SEQ ID NO: 98, a sequence having at least 80%, 85% or 90%, preferably at least 91%, 92%, 93%, 94%, 95%, 96%, 97%, 98% or 99% sequence identity to the sequence set forth in SEQ ID NO: 98, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 98,
the FR-H3 comprises or consists of the amino acid sequence set forth in SEQ ID NO: 99, a sequence having at least 80%, 85% or 90%, preferably at least 91%, 92%, 93%, 94%, 95%, 96%, 97%, 98% or 99% sequence identity to the sequence set forth in SEQ ID NO: 99, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 99,
the FR-H4 comprises or consists of the amino acid sequence set forth in SEQ ID NO: 100, a sequence having at least 80%, 85% or 90%, preferably at least 91%, 92%, 93%, 94%, 95%, 96%, 97%, 98% or 99% sequence identity to the sequence set forth in SEQ ID NO: 100, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 100,
the FR-L1 comprises or consists of the amino acid sequence set forth in SEQ ID NO: 101, a sequence having at least 80%, 85% or 90%, preferably at least 91%, 92%, 93%, 94%, 95%, 96%, 97%, 98% or 99% sequence identity to the sequence set forth in SEQ ID NO: 101, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 101, the FR-L2 comprises or consists of the amino acid sequence set forth in SEQ ID NO: 102, a sequence having at least 80%, 85% or 90%, preferably at least 91%, 92%, 93%, 94%, 95%, 96%, 97%, 98% or 99% sequence identity to the sequence set forth in SEQ ID NO: 102, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 102,
the FR-L3 comprises or consists of the amino acid sequence set forth in SEQ ID NO: 103, a sequence having at least 80%, 85% or 90%, preferably at least 91%, 92%, 93%, 94%, 95%, 96%, 97%, 98% or 99% sequence identity to the sequence set forth in SEQ ID NO: 103, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 103, and
the FR-L4 comprises or consists of the amino acid sequence set forth in SEQ ID NO: 104, a sequence having at least 80%, 85% or 90%, preferably at least 91%, 92%, 93%, 94%, 95%, 96%, 97%, 98% or 99% sequence identity to the sequence set forth in SEQ ID NO: 104, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 104;
(H) the FR-H1 comprises or consists of the amino acid sequence set forth in SEQ ID NO: 105, a sequence having at least 80%, 85% or 90%, preferably at least 91%, 92%, 93%, 94%, 95%, 96%, 97%, 98% or 99% sequence identity to the sequence set forth in SEQ ID NO: 105, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 105,
the FR-H2 comprises or consists of the amino acid sequence set forth in SEQ ID NO: 106, a sequence having at least 80%, 85% or 90%, preferably at least 91%, 92%, 93%, 94%, 95%, 96%, 97%, 98% or 99% sequence identity to the sequence set forth in SEQ ID NO: 106, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 106,
the FR-H3 comprises or consists of the amino acid sequence set forth in SEQ ID NO: 107, a sequence having at least 80%, 85% or 90%, preferably at least 91%, 92%, 93%, 94%, 95%, 96%, 97%, 98% or 99% sequence identity to the sequence set forth in SEQ ID NO: 107, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 107,
the FR-H4 comprises or consists of the amino acid sequence set forth in SEQ ID NO: 108, a sequence having at least 80%, 85% or 90%, preferably at least 91%, 92%, 93%, 94%, 95%, 96%, 97%, 98% or 99% sequence identity to the sequence set forth in SEQ ID NO: 108, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 108,
the FR-L1 comprises or consists of the amino acid sequence set forth in SEQ ID NO: 109, a sequence having at least 80%, 85% or 90%, preferably at least 91%, 92%, 93%, 94%, 95%, 96%, 97%, 98% or 99% sequence identity to the sequence set forth in SEQ ID NO: 109, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 109,
the FR-L2 comprises or consists of the amino acid sequence set forth in SEQ ID NO: 110, a sequence having at least 80%, 85% or 90%, preferably at least 91%, 92%, 93%, 94%, 95%, 96%, 97%, 98% or 99% sequence identity to the sequence set forth in SEQ ID NO: 110, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 110,
the FR-L3 comprises or consists of the amino acid sequence set forth in SEQ ID NO: 111, a sequence having at least 80%, 85% or 90%, preferably at least 91%, 92%, 93%, 94%, 95%, 96%, 97%, 98% or 99% sequence identity to the sequence set forth in SEQ ID NO: 111, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 111, and
the FR-L4 comprises or consists of the amino acid sequence set forth in SEQ ID NO: 112, a sequence having at least 80%, 85% or 90%, preferably at least 91%, 92%, 93%, 94%, 95%, 96%, 97%, 98% or 99% sequence identity to the sequence set forth in SEQ ID NO: 112, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 112;
(I) the FR-H1 comprises or consists of the amino acid sequence set forth in SEQ ID NO: 113, a sequence having at least 80%, 85% or 90%, preferably at least 91%, 92%, 93%, 94%, 95%, 96%, 97%, 98% or 99% sequence identity to the sequence set forth in SEQ ID NO: 113, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 113, the FR-H2 comprises or consists of the amino acid sequence set forth in SEQ ID NO: 114, a sequence having at least 80%, 85% or 90%, preferably at least 91%, 92%, 93%, 94%, 95%, 96%, 97%, 98% or 99% sequence identity to the sequence set forth in SEQ ID NO: 114, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 114,
the FR-H3 comprises or consists of the amino acid sequence set forth in SEQ ID NO: 115, a sequence having at least 80%, 85% or 90%, preferably at least 91%, 92%, 93%, 94%, 95%, 96%, 97%, 98% or 99% sequence identity to the sequence set forth in SEQ ID NO: 115, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 115,
the FR-H4 comprises or consists of the amino acid sequence set forth in SEQ ID NO: 116, a sequence having at least 80%, 85% or 90%, preferably at least 91%, 92%, 93%, 94%, 95%, 96%, 97%, 98% or 99% sequence identity to the sequence set forth in SEQ ID NO: 116, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 116,
the FR-L1 comprises or consists of the amino acid sequence set forth in SEQ ID NO: 117, a sequence having at least 80%, 85% or 90%, preferably at least 91%, 92%, 93%, 94%, 95%, 96%, 97%, 98% or 99% sequence identity to the sequence set forth in SEQ ID NO: 117, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 117,
the FR-L2 comprises or consists of the amino acid sequence set forth in SEQ ID NO: 118, a sequence having at least 80%, 85% or 90%, preferably at least 91%, 92%, 93%, 94%, 95%, 96%, 97%, 98% or 99% sequence identity to the sequence set forth in SEQ ID NO: 118, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 118,
the FR-L3 comprises or consists of the amino acid sequence set forth in SEQ ID NO: 119, a sequence having at least 80%, 85% or 90%, preferably at least 91%, 92%, 93%, 94%, 95%, 96%, 97%, 98% or 99% sequence identity to the sequence set forth in SEQ ID NO: 119, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 119, and
the FR-L4 comprises or consists of the amino acid sequence set forth in SEQ ID NO: 120, a sequence having at least 80%, 85% or 90%, preferably at least 91%, 92%, 93%, 94%, 95%, 96%, 97%, 98% or 99% sequence identity to the sequence set forth in SEQ ID NO: 120, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 120; or
(J) the FR-H1 comprises or consists of the amino acid sequence set forth in SEQ ID NO: 136, a sequence having at least 80%, 85% or 90%, preferably at least 91%, 92%, 93%, 94%, 95%, 96%, 97%, 98% or 99% sequence identity to the sequence set forth in SEQ ID NO: 136, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 136,
the FR-H2 comprises or consists of the amino acid sequence set forth in SEQ ID NO: 137, a sequence having at least 80%, 85% or 90%, preferably at least 91%, 92%, 93%, 94%, 95%, 96%, 97%, 98% or 99% sequence identity to the sequence set forth in SEQ ID NO: 137, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 137,
the FR-H3 comprises or consists of the amino acid sequence set forth in SEQ ID NO: 138, a sequence having at least 80%, 85% or 90%, preferably at least 91%, 92%, 93%, 94%, 95%, 96%, 97%, 98% or 99% sequence identity to the sequence set forth in SEQ ID NO: 138, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 138,
the FR-H4 comprises or consists of the amino acid sequence set forth in SEQ ID NO: 139, a sequence having at least 80%, 85% or 90%, preferably at least 91%, 92%, 93%, 94%, 95%, 96%, 97%, 98% or 99% sequence identity to the sequence set forth in SEQ ID NO: 139, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 139,
the FR-L1 comprises or consists of the amino acid sequence set forth in SEQ ID NO: 140, a sequence having at least 80%, 85% or 90%, preferably at least 91%, 92%, 93%, 94%, 95%, 96%, 97%, 98% or 99% sequence identity to the sequence set forth in SEQ ID NO: 140, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 140,
the FR-L2 comprises or consists of the amino acid sequence set forth in SEQ ID NO: 141, a sequence having at least 80%, 85% or 90%, preferably at least 91%, 92%, 93%, 94%, 95%, 96%, 97%, 98% or 99% sequence identity to the sequence set forth in SEQ ID NO: 141, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 141,
the FR-L3 comprises or consists of the amino acid sequence set forth in SEQ ID NO: 142, a sequence having at least 80%, 85% or 90%, preferably at least 91%, 92%, 93%, 94%, 95%, 96%, 97%, 98% or 99% sequence identity to the sequence set forth in SEQ ID NO: 142, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 142, and
the FR-L4 comprises or consists of the amino acid sequence set forth in SEQ ID NO: 143, a sequence having at least 80%, 85% or 90%, preferably at least 91%, 92%, 93%, 94%, 95%, 96%, 97%, 98% or 99% sequence identity to the sequence set forth in SEQ ID NO: 143, or an amino acid sequence having one or more (preferably 1, 2, 3, 4, 5, 6, 7, 8, 9 or 10) conservative amino acid mutations (preferably substitutions, insertions or deletions) compared to the amino acid sequence set forth in SEQ ID NO: 143.

4. (canceled)

5. (canceled)

6. The antibody or the antigen-binding fragment thereof according to claim 1, wherein the antibody is a humanized antibody, a chimeric antibody or a multispecific antibody (e.g., bispecific antibody).

7. The antibody or the antigen-binding fragment thereof according to claim 6, wherein the constant region of the antibody is humanized, preferably derived from human IgG, more preferably IgG4.

8. The antibody or the antigen-binding fragment thereof according to claim 1, wherein the heavy chain constant region of the antibody is an Ig gamma-4 chain C region, more preferably the Ig gamma-4 chain C region of GenBank ACCESSION No: P01861.1; the light chain constant region is an Ig kappa chain C region, more preferably the Ig kappa chain C region of GenBank ACCESSION No: P01834.

9. The antibody or the antigen-binding fragment thereof according to claim 1, wherein the antigen-binding fragment is selected from Fab, Fab′, F(ab′)2, Fd, Fv, dAb, Fab/c, complementarity determining region (CDR) fragment, single chain antibody (e.g., scFv), bivalent antibody and domain antibody.

10. (canceled)

11. (canceled)

12. (canceled)

13. (canceled)

14. (canceled)

15. (canceled)

16. (canceled)

17. (canceled)

18. A pharmaceutical composition comprising the antibody or antigen-binding fragment thereof according to claim 1, an antibody conjugate a multispecific antibody a fusion protein, and optionally, a pharmaceutically acceptable carrier and/or excipient,
wherein the antibody conjugate comprises the antibody or the antigen-binding fragment thereof according to claim 1 and a conjugated moiety coupled thereto, preferably the conjugated moiety is selected from a purification tag (e.g., His-tag), a cytotoxic agent, a detectable label, a radioisotope, a luminescent substance, a colored substance, an enzyme or polyethylene glycol,
wherein the multispecific antibody (preferably a bispecific antibody) comprises the antibody or the antigen-binding fragment thereof according to claim 1, and an antibody or an antigen-binding fragment against another antigen and/or to another antigenic epitope, and
wherein the fusion protein comprises the antibody or the antigen-binding fragment thereof according to claim 1.

19. The pharmaceutical composition according to claim 8, wherein the pharmaceutical composition is used alone or in combination with one or more medicaments.

20. The pharmaceutical composition according to claim 8, wherein the pharmaceutical composition is in a dosage form suitable for oral administration to the gastrointestinal (GI) tract, preferably the dosage form being selected from tablets, capsules, pills, powders, granules, emulsions, microemulsions, solutions, suspensions, syrups and elixirs; or the pharmaceutical composition is in a dosage form suitable for subcutaneous injection, intradermal injection, intravenous injection, intramuscular injection or intralesional injection.

21. A kit comprising the antibody or the antigen-binding fragment thereof according to claim 1, an antibody conjugate, a multispecific antibody, or a fusion protein, wherein preferably the kit further comprises a second antibody that specifically recognizes the antibody or the antigen-binding fragment thereof claim 1, an antibody, a multispecific antibody or a fusion protein; optionally, the second antibody further comprises a detectable label, such as a radioisotope, a luminescent substance, a colored substance, or an enzyme,
wherein the antibody conjugate comprises the antibody or the antigen-binding fragment thereof according to claim 1 and a conjugated moiety coupled thereto, preferably the conjugated moiety is selected from a purification tag (e.g., His-tag), a cytotoxic agent, a detectable label, a radioisotope, a luminescent substance, a colored substance, an enzyme or polyethylene glycol,
wherein the multispecific antibody (preferably a bispecific antibody) comprises the antibody or the antigen-binding fragment thereof according to claim 1, and an antibody or an antigen-binding fragment against another antigen and/or to another antigenic epitope, and
wherein the fusion protein comprises the antibody or the antigen-binding fragment thereof according to claim 1.

22. (canceled)

23. (canceled)

24. (canceled)

25. (canceled)

26. A method for the prevention and/or treatment and/or adjuvant treatment and/or diagnosis of an allergic disease, a tumor, an autoimmune disease, tissue fibrosis, rhinosinusitis, nasal polyps, chronic obstructive pulmonary disease, comprising administering the antibody or the antigen-binding fragment thereof according to claim 1, an antibody conjugate, a multispecific antibody or a fusion protein to a subject in need, wherein preferably, the allergic disease is selected from atopic dermatitis, allergic rhinitis, asthma and allergy, and more preferably atopic dermatitis includes moderate and severe atopic dermatitis,
wherein the antibody conjugate comprises the antibody or the antigen-binding fragment thereof according to claim 1 and a conjugated moiety coupled thereto, preferably the conjugated moiety is selected from a purification tag (e.g., His-tag), a cytotoxic agent, a detectable label, a radioisotope, a luminescent substance, a colored substance, an enzyme or polyethylene glycol,
wherein the multispecific antibody (preferably a bispecific antibody) comprises the antibody or the antigen-binding fragment thereof according to claim 1, and an antibody or an antigen-binding fragment against another antigen and/or to another antigenic epitope, and
wherein the fusion protein comprises the antibody or the antigen-binding fragment thereof according to claim 1.

27. A hybridoma cell having the accession number of CCTCC NO: C202010, CCTCC NO: C2018131 or CCTCC NO: C2018132.

28. (canceled)


SEQUENCE LISTING (143 sequences)

SEQ ID NO: 1 - DNA, 348 bp, Artificial Sequence
  Artificially Synthesized Sequence
gaggtgcagctgcagcagagcggaccagagctggtgaagcctggcgcctctgtgaagatcagctgtaagacctccggctacaccttcacagagtatacaatccactgggtgaagcagaaccacggcaagagcctggagtggatcggcggcatcaatcctaacaatggcggcaccgtgtacaaccagaacttcaagggcaaggccaccctgacagtggacaagagctcctctaccgcctatatggagctgaggtctctgacaagcgaggactccgccgtgtactattgcgccagagtgcggagaggcatggattactggggccagggcacctccgtgacagtgagctcc

SEQ ID NO: 2 - protein, 116 aa, Artificial Sequence
  Artificially Synthesized Sequence
EVQLQQSGPELVKPGASVKISCKTSGYTFTEYTIHWVKQNHGKSLEWIGGINPNNGGTVYNQNFKGKATLTVDKSSSTAYMELRSLTSEDSAVYYCARVRRGMDYWGQGTSVTVSS

SEQ ID NO: 3 - DNA, 321 bp, Artificial Sequence
  Artificially Synthesized Sequence
gacatcgtgatgacccagtcccacaagtttatgtccacatctgtgggcgacagggtgtccatcacctgtaaggcctctcaggatgtgaccacagccgtggcctggtaccagcagaagccaggccagtctcccaagctgctgatctatagcgcctcctaccggtatacaggcgtgcccgacagattcaccggctctggcagcggcacagatttcacctttacaatcagctccgtgcaggcagaggacctggccgtgtactattgccagcagcactactctgccccttggaccttcggcggaggaacaaacctggagatcaag

SEQ ID NO: 4 - protein, 107 aa, Artificial Sequence
  Artificially Synthesized Sequence
DIVMTQSHKFMSTSVGDRVSITCKASQDVTTAVAWYQQKPGQSPKLLIYSASYRYTGVPDRFTGSGSGTDFTFTISSVQAEDLAVYYCQQHYSAPWTFGGGTNLEIK

SEQ ID NO: 5 - DNA, 360 bp, Artificial Sequence
  Artificially Synthesized Sequence
gatgtgaagctggtggagtccggcggcgacctggtgaacctgggcggaagcctgaagctgagctgtgccgccagcggcttcacattttcctccagctacatgagctgggtgaggcagacacccgagaggagactggagctggtggccgccatcaacagcaatggcggcaagacatactaccctgataccgtgaagggcagattcaccatcagcagggacaacgccaagaacaccctgtacctgcagatgtccggcctgaagacagaggacacagccctgtactactgcaccaggcagaggggcaactacgtgggcgccatggactactggggccagggcacaagcgtgaccgtgagctcc

SEQ ID NO: 6 - protein, 120 aa, Artificial Sequence
  Artificially Synthesized Sequence
DVKLVESGGDLVNLGGSLKLSCAASGFTFSSSYMSWVRQTPERRLELVAAINSNGGKTYYPDTVKGRFTISRDNAKNTLYLQMSGLKTEDTALYYCTRQRGNYVGAMDYWGQGTSVTVSS

SEQ ID NO: 7 - DNA, 321 bp, Artificial Sequence
  Artificially Synthesized Sequence
gacatcgtgatgacacagtcccacaagtttatgtccacatccgtgggcgatagggtgtccatcacatgcaaggccagccaggacgtgagcacagccgtggtgtggtaccagcagaagcctggccagagccccaccctgctgatctactccgccagctacaggtacacaggcgtgcctgacagattcacaggctccggcagcggcaccgatttcacctttaccatctcctccgtgcaggccgaggatctggccgtgtactactgccaccagtactacggcagcccccccacattcggcggcggaacaaagctggagatcaag

SEQ ID NO: 8 - protein, 107 aa, Artificial Sequence
  Artificially Synthesized Sequence
DIVMTQSHKFMSTSVGDRVSITCKASQDVSTAVVWYQQKPGQSPTLLIYSASYRYTGVPDRFTGSGSGTDFTFTISSVQAEDLAVYYCHQYYGSPPTFGGGTKLEIK

SEQ ID NO: 9 - protein, 8 aa, Artificial Sequence
  Artificially Synthesized Sequence
GYTFTEYT

SEQ ID NO: 10 - protein, 8 aa, Artificial Sequence
  Artificially Synthesized Sequence
INPNNGGT

SEQ ID NO: 11 - protein, 9 aa, Artificial Sequence
  Artificially Synthesized Sequence
ARVRRGMDY

SEQ ID NO: 12 - protein, 6 aa, Artificial Sequence
  Artificially Synthesized Sequence
QDVTTA

SEQ ID NO: 13 - protein, 3 aa, Artificial Sequence
  Artificially Synthesized Sequence
SAS

SEQ ID NO: 14 - protein, 9 aa, Artificial Sequence
  Artificially Synthesized Sequence
QQHYSAPWT

SEQ ID NO: 15 - protein, 8 aa, Artificial Sequence
  Artificially Synthesized Sequence
GFTFSSSY

SEQ ID NO: 16 - protein, 8 aa, Artificial Sequence
  Artificially Synthesized Sequence
INSNGGKT

SEQ ID NO: 17 - protein, 13 aa, Artificial Sequence
  Artificially Synthesized Sequence
TRQRGNYVGAMDY

SEQ ID NO: 18 - protein, 6 aa, Artificial Sequence
  Artificially Synthesized Sequence
QDVSTA

SEQ ID NO: 19 - protein, 3 aa, Artificial Sequence
  Artificially Synthesized Sequence
SAS

SEQ ID NO: 20 - protein, 9 aa, Artificial Sequence
  Artificially Synthesized Sequence
HQYYGSPPT

SEQ ID NO: 21 - DNA, 348 bp, Artificial Sequence
  Artificially Synthesized Sequence
caggtgcagctgcagcagtccggagcagaggtggtgaagccaggagccagcgtgaagatctcctgtaagacctctggctacaccttcacagagtatacaatccactgggtgaagcaggcacacggacagagcctggagtggatcggcggcatcaaccctaacaatggcggcaccgtgtacaatcagaagtttcagggcaaggccaccctgacagtggacaagtctaccagcacagcctatatggagctgaggtccctgacctctgaggacacagccgtgtactattgcgcccgggtgcggagaggcatggattactggggccagggcacctccgtgacagtgagctcc

SEQ ID NO: 22 - protein, 116 aa, Artificial Sequence
  Artificially Synthesized Sequence
QVQLQQSGAEVVKPGASVKISCKTSGYTFTEYTIHWVKQAHGQSLEWIGGINPNNGGTVYNQKFQGKATLTVDKSTSTAYMELRSLTSEDTAVYYCARVRRGMDYWGQGTSVTVSS

SEQ ID NO: 23 - DNA, 321 bp, Artificial Sequence
  Artificially Synthesized Sequence
gacatccagatgacccagtctcctaagtctctgagcacatccgtgggcgaccgggtgaccatcacatgtagagccagccaggatgtgaccacagcagtggcatggtaccagcagaagcctggcaagtcccctaagctgctgatctattctgccagctacaggtataccggagtgccatctcggttctccggctctggcagcggcacagacttcacctttacaatcagctccgtgcagccagaggatctggccacctactattgccagcagcactacagcgccccatggacctttggcggaggaacaaacctggagatcaag

SEQ ID NO: 24 - protein, 107 aa, Artificial Sequence
  Artificially Synthesized Sequence
DIQMTQSPKSLSTSVGDRVTITCRASQDVTTAVAWYQQKPGKSPKLLIYSASYRYTGVPSRFSGSGSGTDFTFTISSVQPEDLATYYCQQHYSAPWTFGGGTNLEIK

SEQ ID NO: 25 - DNA, 348 bp, Artificial Sequence
  Artificially Synthesized Sequence
caggtgcagctggtgcagtccggagcagaggtggtgaagccaggagccagcgtgaaggtgtcctgtaagacctctggctacaccttcacagagtatacaatccactgggtgcggcaggcaccaggacagtctctggagtggatcggcggcatcaaccctaacaatggcggcaccaactacaatcagaagtttcagggcaaggtgaccctgacagtggacaagtccacctctacagcctatatggagctgagctccctgaggtctgaggacacagccgtgtactattgcgcccgcgtgcggagaggcatggattactggggccagggcaccagcgtgacagtgtctagc

SEQ ID NO: 26 - protein, 116 aa, Artificial Sequence
  Artificially Synthesized Sequence
QVQLVQSGAEVVKPGASVKVSCKTSGYTFTEYTIHWVRQAPGQSLEWIGGINPNNGGTNYNQKFQGKVTLTVDKSTSTAYMELSSLRSEDTAVYYCARVRRGMDYWGQGTSVTVSS

SEQ ID NO: 27 - DNA, 321 bp, Artificial Sequence
  Artificially Synthesized Sequence
gacatccagatgacccagagccctagctccctgagcgcctccgtgggcgacagggtgaccatcacatgtagagcctcccaggatgtgaccacagcagtggcatggtaccagcagaagccaggcaaggcccctaagctgctgatctactccgcctctagcaggtataccggagtgccatctcggttctctggcagcggctccggcacagactttaccctgacaatctcctctgtgcagccagaggatctggccacatactattgccagcagcactattctgccccctggacctttggcggcggcacaaacctggagatcaag

SEQ ID NO: 28 - protein, 107 aa, Artificial Sequence
  Artificially Synthesized Sequence
DIQMTQSPSSLSASVGDRVTITCRASQDVTTAVAWYQQKPGKAPKLLIYSASSRYTGVPSRFSGSGSGTDFTLTISSVQPEDLATYYCQQHYSAPWTFGGGTNLEIK

SEQ ID NO: 29 - DNA, 348 bp, Artificial Sequence
  Artificially Synthesized Sequence
caggtgcagctggtgcagtccggagcagaggtggtgaagccaggagccagcgtgaaggtgtcctgtaaggcctctggctacaccttcacagagtataccatccactgggtgcggcaggcaccaggacagggactggagtggatcggcggcatcaaccctaacaatggcggcacaaattacgcccagaagtttcagggcagggtgaccatcacagtggacaagtccacctctacagcctatatggagctgagctccctgaggtctgaggacaccgccgtgtactattgcgcccgcgtgcggagaggcatggattactggggccagggcaccagcgtgacagtgtctagc

SEQ ID NO: 30 - protein, 116 aa, Artificial Sequence
  Artificially Synthesized Sequence
QVQLVQSGAEVVKPGASVKVSCKASGYTFTEYTIHWVRQAPGQGLEWIGGINPNNGGTNYAQKFQGRVTITVDKSTSTAYMELSSLRSEDTAVYYCARVRRGMDYWGQGTSVTVSS

SEQ ID NO: 31 - DNA, 321 bp, Artificial Sequence
  Artificially Synthesized Sequence
gacatccagatgacccagagccctagctccctgagcgcctccgtgggcgacagggtgaccatcacatgtagagcctcccaggatgtgaccacagccctggcatggtaccagcagaagccaggcaaggcccctaagctgctgatctactccgcctctagcctgtataccggagtgccatctcggttctctggcagcggctccggcacagactttaccctgacaatctcctctctgcagccagaggatttcgccacatactattgccagcagcactattctgccccctggacctttggcggcggcacaaacctggagatcaag

SEQ ID NO: 32 - protein, 107 aa, Artificial Sequence
  Artificially Synthesized Sequence
DIQMTQSPSSLSASVGDRVTITCRASQDVTTALAWYQQKPGKAPKLLIYSASSLYTGVPSRFSGSGSGTDFTLTISSLQPEDFATYYCQQHYSAPWTFGGGTNLEIK

SEQ ID NO: 33 - DNA, 348 bp, Artificial Sequence
  Artificially Synthesized Sequence
caggtgcagctggtgcagtccggagcagaggtggtgaagccaggagccagcgtgaaggtgtcctgtaagacctctggctacaccttcacagagtatacaatccactgggtgcggcaggcaccaggacagtctctggagtggatcggcggcatcaaccctaacaatggcggcaccgtctacaatcagaagtttcagggcaaggtgaccctgacagtggacaagtccacctctacagcctatatggagctgagctccctgaggtctgaggacacagccgtgtactattgcgcccgcgtgcggagaggcatggattactggggccagggcaccagcgtgacagtgtctagc

SEQ ID NO: 34 - protein, 116 aa, Artificial Sequence
  Artificially Synthesized Sequence
QVQLVQSGAEVVKPGASVKVSCKTSGYTFTEYTIHWVRQAPGQSLEWIGGINPNNGGTVYNQKFQGKVTLTVDKSTSTAYMELSSLRSEDTAVYYCARVRRGMDYWGQGTSVTVSS

SEQ ID NO: 35 - DNA, 321 bp, Artificial Sequence
  Artificially Synthesized Sequence
gacatccagatgacccagagccctagctccctgagcgcctccgtgggcgacagggtgaccatcacatgtagagcctcccaggatgtgaccacagcagtggcatggtaccagcagaagccaggcaaggcccctaagctgctgatctactccgcctcttacaggtataccggagtgccatctcggttctctggcagcggctccggcacagactttaccctgacaatctcctctgtgcagccagaggatctggccacatactattgccagcagcactattctgccccctggacctttggcggcggcacaaacctggagatcaag

SEQ ID NO: 36 - protein, 107 aa, Artificial Sequence
  Artificially Synthesized Sequence
DIQMTQSPSSLSASVGDRVTITCRASQDVTTAVAWYQQKPGKAPKLLIYSASYRYTGVPSRFSGSGSGTDFTLTISSVQPEDLATYYCQQHYSAPWTFGGGTNLEIK

SEQ ID NO: 37 - DNA, 360 bp, Artificial Sequence
  Artificially Synthesized Sequence
gacgtgcagctggtggagagcggcggcggcctggtgcagctgggcggctccctgaggctgtcttgtgcagccagcggcttcacctttagctcctcttacatgtcttgggtgcggcagacacccgagaagagactggagctggtggccgccatcaactctaatggcggcaagacctactatgccgacagcgtgaagggcaggtttaccatctcccgcgataactctaagaatacactgtatctgcagatgagctccctgagagccgaggacaccgccgtgtactattgcacacggcagagaggcaactacgtgggcgccatggattattggggccagggcaccagcgtgacagtgtctagc

SEQ ID NO: 38 - protein, 120 aa, Artificial Sequence
  Artificially Synthesized Sequence
DVQLVESGGGLVQLGGSLRLSCAASGFTFSSSYMSWVRQTPEKRLELVAAINSNGGKTYYADSVKGRFTISRDNSKNTLYLQMSSLRAEDTAVYYCTRQRGNYVGAMDYWGQGTSVTVSS

SEQ ID NO: 39 - DNA, 321 bp, Artificial Sequence
  Artificially Synthesized Sequence
gacatccagatgacccagtctcccagctccctgtccacatctgtgggcgaccgggtgaccatcacctgtaaggcctctcaggatgtgagcaccgccgtggtgtggtaccagcagaagccaggcaagagccccaagctgctgatctatagcgcctcctaccggtataccggcgtgccttccagattctctggcagcggctccggcacagacttcacctttacaatctctagcgtgcagccagaggatatcgccacatactattgccaccagtactatggcagccctcccaccttcggcggcggcacaaagctggagatcaag

SEQ ID NO: 40 - protein, 107 aa, Artificial Sequence
  Artificially Synthesized Sequence
DIQMTQSPSSLSTSVGDRVTITCKASQDVSTAVVWYQQKPGKSPKLLIYSASYRYTGVPSRFSGSGSGTDFTFTISSVQPEDIATYYCHQYYGSPPTFGGGTKLEIK

SEQ ID NO: 41 - DNA, 360 bp, Artificial Sequence
  Artificially Synthesized Sequence
gaggtgcagctggtggagagcggcggcggcctggtgcagcccggcggctccctgaggctgtcttgtgccgccagcggcttcacctttagctcctcttacatgtcttgggtgcggcaggcacctggcaagagactggagctggtggccgccatcaactctaatggcggcaagacatactatgccgacagcgtgaagggcaggtttaccatctcccgcgataactctaagaatacactgtatctgcagatgagctccctgagagccgaggacaccgccgtgtactattgcacacggcagagaggcaactacgtgggcgccatggattattggggccagggcaccagcgtgacagtgtctagc

SEQ ID NO: 42 - protein, 120 aa, Artificial Sequence
  Artificially Synthesized Sequence
EVQLVESGGGLVQPGGSLRLSCAASGFTFSSSYMSWVRQAPGKRLELVAAINSNGGKTYYADSVKGRFTISRDNSKNTLYLQMSSLRAEDTAVYYCTRQRGNYVGAMDYWGQGTSVTVSS

SEQ ID NO: 43 - DNA, 321 bp, Artificial Sequence
  Artificially Synthesized Sequence
gacatccagatgacccagtctccaagctccctgtccgcctctgtgggcgacagggtgaccatcacatgtcaggcctctcaggatgtgagcacagccgtggtgtggtaccagcagaagccaggcaagagccccaagctgctgatctatagcgcctcctacctgtataccggcgtgccttccagattctctggcagcggctccggcacagacttcacctttacaatctctagcctgcagccagaggatatcgccacctactattgccaccagtactatggcagccctcccaccttcggcggcggcacaaagctggagatcaag

SEQ ID NO: 44 - protein, 107 aa, Artificial Sequence
  Artificially Synthesized Sequence
DIQMTQSPSSLSASVGDRVTITCQASQDVSTAVVWYQQKPGKSPKLLIYSASYLYTGVPSRFSGSGSGTDFTFTISSLQPEDIATYYCHQYYGSPPTFGGGTKLEIK

SEQ ID NO: 45 - DNA, 360 bp, Artificial Sequence
  Artificially Synthesized Sequence
gaggtgcagctggtggagagcggcggcggcctggtgcagcccggcggctccctgcggctgtcttgtgccgccagcggcttcacctttagctcctcttacatgtcctgggtgaggcaggcacctggcaagggactggagtacgtgagcgccatcaactctaatggcggcaagacatactatgccgactctgtgaagggcaggtttaccatcagccgcgataactccaagaatacactgtacctgcagatgagctccctgagggcagaggacaccgccgtgtactattgcacacggcagagaggcaactacgtgggcgccatggattattggggccagggcacctccgtgacagtgtctagc

SEQ ID NO: 46 - protein, 120 aa, Artificial Sequence
  Artificially Synthesized Sequence
EVQLVESGGGLVQPGGSLRLSCAASGFTFSSSYMSWVRQAPGKGLEYVSAINSNGGKTYYADSVKGRFTISRDNSKNTLYLQMSSLRAEDTAVYYCTRQRGNYVGAMDYWGQGTSVTVSS

SEQ ID NO: 47 - DNA, 321 bp, Artificial Sequence
  Artificially Synthesized Sequence
gacatccagatgacccagtctccaagctccctgtccgcctctgtgggcgacagggtgaccatcacatgtcaggcctctcaggatgtgagcacagccctggcctggtaccagcagaagccaggcaaggcccccaagctgctgatctatagcgcctccaacctggagaccggcgtgcctagcagattctctggcagcggctccggcacagacttcacctttacaatctctagcctgcagccagaggatatcgccacctactattgccaccagtactatggcagccctcccaccttcggcggcggcacaaagctggagatcaag

SEQ ID NO: 48 - protein, 107 aa, Artificial Sequence
  Artificially Synthesized Sequence
DIQMTQSPSSLSASVGDRVTITCQASQDVSTALAWYQQKPGKAPKLLIYSASNLETGVPSRFSGSGSGTDFTFTISSLQPEDIATYYCHQYYGSPPTFGGGTKLEIK

SEQ ID NO: 49 - protein, 25 aa, Artificial Sequence
  Artificially Synthesized Sequence
EVQLQQSGPELVKPGASVKISCKTS

SEQ ID NO: 50 - protein, 17 aa, Artificial Sequence
  Artificially Synthesized Sequence
IHWVKQNHGKSLEWIGG

SEQ ID NO: 51 - protein, 38 aa, Artificial Sequence
  Artificially Synthesized Sequence
VYNQNFKGKATLTVDKSSSTAYMELRSLTSEDSAVYYC

SEQ ID NO: 52 - protein, 11 aa, Artificial Sequence
  Artificially Synthesized Sequence
WGQGTSVTVSS

SEQ ID NO: 53 - protein, 26 aa, Artificial Sequence
  Artificially Synthesized Sequence
DIVMTQSHKFMSTSVGDRVSITCKAS

SEQ ID NO: 54 - protein, 17 aa, Artificial Sequence
  Artificially Synthesized Sequence
VAWYQQKPGQSPKLLIY

SEQ ID NO: 55 - protein, 36 aa, Artificial Sequence
  Artificially Synthesized Sequence
YRYTGVPDRFTGSGSGTDFTFTISSVQAEDLAVYYC

SEQ ID NO: 56 - protein, 10 aa, Artificial Sequence
  Artificially Synthesized Sequence
FGGGTNLEIK

SEQ ID NO: 57 - protein, 25 aa, Artificial Sequence
  Artificially Synthesized Sequence
DVKLVESGGDLVNLGGSLKLSCAAS

SEQ ID NO: 58 - protein, 17 aa, Artificial Sequence
  Artificially Synthesized Sequence
MSWVRQTPERRLELVAA

SEQ ID NO: 59 - protein, 38 aa, Artificial Sequence
  Artificially Synthesized Sequence
YYPDTVKGRFTISRDNAKNTLYLQMSGLKTEDTALYYC

SEQ ID NO: 60 - protein, 11 aa, Artificial Sequence
  Artificially Synthesized Sequence
WGQGTSVTVSS

SEQ ID NO: 61 - protein, 26 aa, Artificial Sequence
  Artificially Synthesized Sequence
DIVMTQSHKFMSTSVGDRVSITCKAS

SEQ ID NO: 62 - protein, 17 aa, Artificial Sequence
  Artificially Synthesized Sequence
VVWYQQKPGQSPTLLIY

SEQ ID NO: 63 - protein, 36 aa, Artificial Sequence; identical to SEQ ID NO: 55
  Artificially Synthesized Sequence

SEQ ID NO: 64 - protein, 10 aa, Artificial Sequence
  Artificially Synthesized Sequence
FGGGTKLEIK

SEQ ID NO: 65 - protein, 25 aa, Artificial Sequence
  Artificially Synthesized Sequence
QVQLQQSGAEVVKPGASVKISCKTS

SEQ ID NO: 66 - protein, 17 aa, Artificial Sequence
  Artificially Synthesized Sequence
IHWVKQAHGQSLEWIGG

SEQ ID NO: 67 - protein, 38 aa, Artificial Sequence
  Artificially Synthesized Sequence
VYNQKFQGKATLTVDKSTSTAYMELRSLTSEDTAVYYC

SEQ ID NO: 68 - protein, 11 aa, Artificial Sequence
  Artificially Synthesized Sequence
WGQGTSVTVSS

SEQ ID NO: 69 - protein, 26 aa, Artificial Sequence
  Artificially Synthesized Sequence
DIQMTQSPKSLSTSVGDRVTITCRAS

SEQ ID NO: 70 - protein, 17 aa, Artificial Sequence
  Artificially Synthesized Sequence
VAWYQQKPGKSPKLLIY

SEQ ID NO: 71 - protein, 36 aa, Artificial Sequence
  Artificially Synthesized Sequence
YRYTGVPSRFSGSGSGTDFTFTISSVQPEDLATYYC

SEQ ID NO: 72 - protein, 10 aa, Artificial Sequence
  Artificially Synthesized Sequence
FGGGTNLEIK

SEQ ID NO: 73 - protein, 25 aa, Artificial Sequence
  Artificially Synthesized Sequence
QVQLVQSGAEVVKPGASVKVSCKTS

SEQ ID NO: 74 - protein, 17 aa, Artificial Sequence
  Artificially Synthesized Sequence
IHWVRQAPGQSLEWIGG

SEQ ID NO: 75 - protein, 38 aa, Artificial Sequence
  Artificially Synthesized Sequence
NYNQKFQGKVTLTVDKSTSTAYMELSSLRSEDTAVYYC

SEQ ID NO: 76 - protein, 11 aa, Artificial Sequence
  Artificially Synthesized Sequence
WGQGTSVTVSS

SEQ ID NO: 77 - protein, 26 aa, Artificial Sequence [sequence omitted]

SEQ ID NO: 78 - protein, 17 aa, Artificial Sequence
  Artificially Synthesized Sequence
VAWYQQKPGKAPKLLIY

SEQ ID NO: 79 - protein, 36 aa, Artificial Sequence
  Artificially Synthesized Sequence
SRYTGVPSRFSGSGSGTDFTLTISSVQPEDLATYYC

SEQ ID NO: 80 - protein, 10 aa, Artificial Sequence
  Artificially Synthesized Sequence
FGGGTNLEIK

SEQ ID NO: 81 - protein, 25 aa, Artificial Sequence
  Artificially Synthesized Sequence
QVQLVQSGAEVVKPGASVKVSCKAS

SEQ ID NO: 82 - protein, 17 aa, Artificial Sequence
  Artificially Synthesized Sequence
IHWVRQAPGQGLEWIGG

SEQ ID NO: 83 - protein, 38 aa, Artificial Sequence
  Artificially Synthesized Sequence
NYAQKFQGRVTITVDKSTSTAYMELSSLRSEDTAVYYC

SEQ ID NO: 84 - protein, 11 aa, Artificial Sequence
  Artificially Synthesized Sequence
WGQGTSVTVSS

SEQ ID NO: 85 - protein, 26 aa, Artificial Sequence [sequence omitted]

SEQ ID NO: 86 - protein, 17 aa, Artificial Sequence
  Artificially Synthesized Sequence
LAWYQQKPGKAPKLLIY

SEQ ID NO: 87 - protein, 36 aa, Artificial Sequence
  Artificially Synthesized Sequence
SLYTGVPSRFSGSGSGTDFTLTISSLQPEDFATYYC

SEQ ID NO: 88 - protein, 10 aa, Artificial Sequence
  Artificially Synthesized Sequence
FGGGTNLEIK

SEQ ID NO: 89 - protein, 25 aa, Artificial Sequence
  Artificially Synthesized Sequence
QVQLVQSGAEVVKPGASVKVSCKTS

SEQ ID NO: 90 - protein, 17 aa, Artificial Sequence
  Artificially Synthesized Sequence
IHWVRQAPGQSLEWIGG

SEQ ID NO: 91 - protein, 38 aa, Artificial Sequence
  Artificially Synthesized Sequence
VYNQKFQGKVTLTVDKSTSTAYMELSSLRSEDTAVYYC

SEQ ID NO: 92 - protein, 11 aa, Artificial Sequence
  Artificially Synthesized Sequence
WGQGTSVTVSS

SEQ ID NO: 93 - protein, 26 aa, Artificial Sequence [sequence omitted]

SEQ ID NO: 94 - protein, 17 aa, Artificial Sequence
  Artificially Synthesized Sequence
VAWYQQKPGKAPKLLIY

SEQ ID NO: 95 - protein, 36 aa, Artificial Sequence
  Artificially Synthesized Sequence
YRYTGVPSRFSGSGSGTDFTLTISSVQPEDLATYYC

SEQ ID NO: 96 - protein, 10 aa, Artificial Sequence
  Artificially Synthesized Sequence
FGGGTNLEIK

SEQ ID NO: 97 - protein, 25 aa, Artificial Sequence
  Artificially Synthesized Sequence
DVQLVESGGGLVQLGGSLRLSCAAS

SEQ ID NO: 98 - protein, 17 aa, Artificial Sequence
  Artificially Synthesized Sequence
MSWVRQTPEKRLELVAA

SEQ ID NO: 99 - protein, 38 aa, Artificial Sequence
  Artificially Synthesized Sequence
YYADSVKGRFTISRDNSKNTLYLQMSSLRAEDTAVYYC

SEQ ID NO: 100 - protein, 11 aa, Artificial Sequence
  Artificially Synthesized Sequence
WGQGTSVTVSS

SEQ ID NO: 101 - protein, 26 aa, Artificial Sequence
  Artificially Synthesized Sequence
DIQMTQSPSSLSTSVGDRVTITCKAS

SEQ ID NO: 102 - protein, 17 aa, Artificial Sequence
  Artificially Synthesized Sequence
VVWYQQKPGKSPKLLIY

SEQ ID NO: 103 - protein, 36 aa, Artificial Sequence
  Artificially Synthesized Sequence
YRYTGVPSRFSGSGSGTDFTFTISSVQPEDIATYYC

SEQ ID NO: 104 - protein, 10 aa, Artificial Sequence
  Artificially Synthesized Sequence
FGGGTKLEIK

SEQ ID NO: 105 - protein, 25 aa, Artificial Sequence [sequence omitted]

SEQ ID NO: 106 - protein, 17 aa, Artificial Sequence
  Artificially Synthesized Sequence
MSWVRQAPGKRLELVAA

SEQ ID NO: 107 - protein, 38 aa, Artificial Sequence; identical to SEQ ID NO: 99
  Artificially Synthesized Sequence

SEQ ID NO: 108 - protein, 11 aa, Artificial Sequence
  Artificially Synthesized Sequence
WGQGTSVTVSS

SEQ ID NO: 109 - protein, 26 aa, Artificial Sequence
  Artificially Synthesized Sequence
DIQMTQSPSSLSASVGDRVTITCQAS

SEQ ID NO: 110 - protein, 17 aa, Artificial Sequence
  Artificially Synthesized Sequence
VVWYQQKPGKSPKLLIY

SEQ ID NO: 111 - protein, 36 aa, Artificial Sequence
  Artificially Synthesized Sequence
YLYTGVPSRFSGSGSGTDFTFTISSLQPEDIATYYC

SEQ ID NO: 112 - protein, 10 aa, Artificial Sequence
  Artificially Synthesized Sequence
FGGGTKLEIK

SEQ ID NO: 113 - protein, 25 aa, Artificial Sequence [sequence omitted]

SEQ ID NO: 114 - protein, 17 aa, Artificial Sequence
  Artificially Synthesized Sequence
MSWVRQAPGKGLEYVSA

SEQ ID NO: 115 - protein, 38 aa, Artificial Sequence; identical to SEQ ID NO: 99
  Artificially Synthesized Sequence

SEQ ID NO: 116 - protein, 11 aa, Artificial Sequence
  Artificially Synthesized Sequence
WGQGTSVTVSS

SEQ ID NO: 117 - protein, 26 aa, Artificial Sequence
  Artificially Synthesized Sequence
DIQMTQSPSSLSASVGDRVTITCQAS

SEQ ID NO: 118 - protein, 17 aa, Artificial Sequence
  Artificially Synthesized Sequence
LAWYQQKPGKAPKLLIY

SEQ ID NO: 119 - protein, 36 aa, Artificial Sequence
  Artificially Synthesized Sequence
NLETGVPSRFSGSGSGTDFTFTISSLQPEDIATYYC

SEQ ID NO: 120 - protein, 10 aa, Artificial Sequence
  Artificially Synthesized Sequence
FGGGTKLEIK

SEQ ID NO: 121 - protein, 232 aa, Artificial Sequence
  Artificially Synthesized Sequence
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 122 - protein, 443 aa, Artificial Sequence
  Artificially Synthesized Sequence
EVQLEQSGAELMKPGASVKISCKATGYTFTTYWIEWIKQRPGHSLEWIGEILPGSDSTYYNEKVKGKVTFTADASSNTAYMQLSSLTSEDSAVYYCARGDGFYVYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 123 - protein, 214 aa, Artificial Sequence
  Artificially Synthesized Sequence
DIELTQSPATLSVTPGDSVSLSCRASQSISNNLHWYQQKSHESPRLLIKYTSQSMSGIPSRFSGSGSGTDFTLSINSVETEDFGVYFCQQSGSWPRTFGGGTKLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 118 aa, Artificial Sequence
  Artificially Synthesized Sequence
EVQLVESGGGLVQPGRSLRLSCEASGFTFDDYAMHWVRQAPGKGLEWVSGLSRTSVSIGYADSVKGRFTISRDNAKNSLYLEMNSLRPEDTALYYCAKWGTRGYFDYWGQGTLVTVSS

SEQ ID NO: 125 - protein, 107 aa, Artificial Sequence
  Artificially Synthesized Sequence
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 126 - DNA, 360 bp, Artificial Sequence
  Artificially Synthesized Sequence
caggtgacactgaaggagtccggcccaaccctggtgaagcccacccagacactgaccctgacatgtaccttcagcggcttttctctgagcacatccggcatgggagtgggatggatcagacagccccctggcaaggccctggagtggctggccctgatctggtgggccgacgataagaggtactctaccagcctgaagtcccgcctgacaatctccaaggacacctctaagaaccaggtggtgctgacaatcaccaatgtggaccccgtggatacagccacctactattgcgcccggatcacaagaggcaactctgccatggatttttggggccagggcacatctgtgaccgtgagctcc

SEQ ID NO: 127 - protein, 120 aa, Artificial Sequence
  Artificially Synthesized Sequence
QVTLKESGPTLVKPTQTLTLTCTFSGFSLSTSGMGVGWIRQPPGKALEWLALIWWADDKRYSTSLKSRLTISKDTSKNQVVLTITNVDPVDTATYYCARITRGNSAMDFWGQGTSVTVSS

SEQ ID NO: 128 - DNA, 321 bp, Artificial Sequence
  Artificially Synthesized Sequence
gacatccagatgacccagtctcctagctccctgtccgcctctgtgggcgatagggtgaccatcacatgtagagcctctgagaacgtgtacagctatctgaattggtaccagcagaagcccggcaaggcccctaagctgctgatctataacgccaagagcctgcagtccggagtgccaagccggttcagcggctccggctctggcaccgactttaccctgacaatctctagcctgcagccagaggatttcgccacatactattgccagcaccactacggcatcccctggaccttcggcggcggcacaaagctggagatcaag

SEQ ID NO: 129 - protein, 107 aa, Artificial Sequence
  Artificially Synthesized Sequence
DIQMTQSPSSLSASVGDRVTITCRASENVYSYLNWYQQKPGKAPKLLIYNAKSLQSGVPSRFSGSGSGTDFTLTISSLQPEDFATYYCQHHYGIPWTFGGGTKLEIK

SEQ ID NO: 130 - protein, 10 aa, Artificial Sequence
  Artificially Synthesized Sequence
GFSLSTSGMG

SEQ ID NO: 131 - protein, 7 aa, Artificial Sequence
  Artificially Synthesized Sequence
IWWADDK

SEQ ID NO: 132 - protein, 12 aa, Artificial Sequence
  Artificially Synthesized Sequence
ARITRGNSAMDF

SEQ ID NO: 133 - protein, 6 aa, Artificial Sequence
  Artificially Synthesized Sequence
ENVYSY

SEQ ID NO: 134 - protein, 3 aa, Artificial Sequence
  Artificially Synthesized Sequence
NAK

SEQ ID NO: 135 - protein, 9 aa, Artificial Sequence
  Artificially Synthesized Sequence
QHHYGIPWT

SEQ ID NO: 136 - protein, 25 aa, Artificial Sequence
  Artificially Synthesized Sequence
QVTLKESGPTLVKPTQTLTLTCTFS

SEQ ID NO: 137 - protein, 17 aa, Artificial Sequence
  Artificially Synthesized Sequence
VGWIRQPPGKALEWLAL

SEQ ID NO: 138 - protein, 38 aa, Artificial Sequence
  Artificially Synthesized Sequence
RYSTSLKSRLTISKDTSKNQVVLTITNVDPVDTATYYC

SEQ ID NO: 139 - protein, 11 aa, Artificial Sequence
  Artificially Synthesized Sequence
WGQGTSVTVSS

SEQ ID NO: 140 - protein, 26 aa, Artificial Sequence [sequence omitted]

SEQ ID NO: 141 - protein, 17 aa, Artificial Sequence
  Artificially Synthesized Sequence
LNWYQQKPGKAPKLLIY

SEQ ID NO: 142 - protein, 36 aa, Artificial Sequence
  Artificially Synthesized Sequence
SLQSGVPSRFSGSGSGTDFTLTISSLQPEDFATYYC

SEQ ID NO: 143 - protein, 10 aa, Artificial Sequence
  Artificially Synthesized Sequence
FGGGTKLEIK